SEQUENCE LISTING (16 sequences)

SEQ ID NO: 1 - DNA, 22 bp, synthetic construct
  source 1..22: mol_type = other DNA
gatcgccagaaagctgaaggag

SEQ ID NO: 2 - DNA, 22 bp, synthetic construct
  source 1..22: mol_type = other DNA
ccacctcataggctgtttgctc

SEQ ID NO: 3 - DNA, 22 bp, synthetic construct
  source 1..22: mol_type = other DNA
gtgggacaagagcctgagcaat

SEQ ID NO: 4 - DNA, 23 bp, synthetic construct
  source 1..23: mol_type = other DNA
ggatttccagagcggctgtattc

SEQ ID NO: 5 - DNA, 21 bp, synthetic construct
  source 1..21: mol_type = other DNA
cctcgcctccgattgaagatg

SEQ ID NO: 6 - DNA, 21 bp, synthetic construct
  source 1..21: mol_type = other DNA
aaaggcagtcaaatctggtgg

SEQ ID NO: 7 - DNA, 19 bp, synthetic construct
  source 1..19: mol_type = other DNA
ggacaaaccccaaccatcc

SEQ ID NO: 8 - DNA, 22 bp, synthetic construct
  source 1..22: mol_type = other DNA
gttgaactcagacatcgttcct

SEQ ID NO: 9 - DNA, 30 bp, synthetic construct
  source 1..30: mol_type = other DNA
cggactcgcgaagaggaggcccacttctac

SEQ ID NO: 10 - DNA, 29 bp, synthetic construct
  source 1..29: mol_type = other DNA
ccacccgcgtagaagtgggcctcctcttc

SEQ ID NO: 11 - DNA, 22 bp, synthetic construct
  source 1..22: mol_type = other DNA
cctatgaccgagatttcctcgc

SEQ ID NO: 12 - DNA, 22 bp, synthetic construct
  source 1..22: mol_type = other DNA
tgaagctgtccctgcccgtata

SEQ ID NO: 13 - DNA, 22 bp, synthetic construct
  source 1..22: mol_type = other DNA
aggcagaaccagtcccttgtgt

SEQ ID NO: 14 - DNA, 23 bp, synthetic construct
  source 1..23: mol_type = other DNA
ctttgtgctggagccataaacag

SEQ ID NO: 15 - DNA, 22 bp, synthetic construct
  source 1..22: mol_type = other DNA
ctcttaccgcagcctgagtttc

SEQ ID NO: 16 - DNA, 22 bp, synthetic construct
  source 1..22: mol_type = other DNA
gcaggaagatgcaaccattggc

(19) United States
(12) Patent Application Publication
(10) Pub. No.: US 2025/0170239 A1
(43) Pub. Date: May 29, 2025

(54) CELLULAR REPROGRAMMING BY TARGETING THE GOLGI APPARATUS

(71) Applicant: MUSC Foundation for Research Development, Charleston, SC (US)

(72) Inventors: Shikhar Mehrotra, Mt. Pleasant, SC (US); Nathaniel Oberholtzer, Charleston, SC (US)

(21) Appl. No.: 18/934,471

(22) Filed: Nov. 1, 2024

Related U.S. Application Data
(60) Provisional application No. 63/595,965, filed on Nov. 3, 2023.

(51) Int. Cl. A61K 40/11; A61K 40/31; A61K 40/42; A61P 35/00; C12N 5/0783; C12N 9/88; G01N 15/149

(52) U.S. Cl. CPC A61K 40/11; A61K 40/31; A61K 40/42; A61P 35/00; C12N 5/0636; C12N 9/88; C12Y 402/01022; G01N 15/149

(57) ABSTRACT

The present invention generally relates to methods and compositions for the generation of T cells with enhanced features and enhanced therapeutic properties. The present invention also relates to methods of treating or preventing cancer with the T cells which display enhanced features and enhanced therapeutic properties.

### CROSS-REFERENCE TO RELATED APPLICATIONS

This application claims priority to U.S. Provisional Patent Application No. 63/595,965, filed Nov. 3, 2023, which is hereby incorporated herein by reference in its entirety.

### REFERENCE TO A “SEQUENCE LISTING” SUBMITTED AS AN XML FILE

The Sequence Listing written in the XML file: “206085-0163-OOUS_Sequence listing.xml”; created on Nov. 1, 2024, and 15,216 bytes in size, is hereby incorporated by reference.

### BACKGROUND OF THE INVENTION

Immunotherapy is a treatment method that uses the immune system to help fight against cancers and diseases, and includes monoclonal antibodies, immune checkpoint inhibitors, cancer vaccines, and adoptive T cell therapy (ACT). T cells are white blood cells that originate in the bone marrow and mature in the thymus and tonsils. T cells are responsible for suppressing the immune system, protecting the body from viruses and infections, and can alert the body by sending chemical messages to activated T cells. Adoptive T cell therapy involves isolating subject T cells, expanding and/or manipulating them ex vivo to be a more effective therapeutic, and administering them to the subject. ACT is a promising approach for treating patients with advanced malignancies (Rosenberg, S. A. et al., 2012, Sci Transl Med, 4(127):127ps8; Kershaw, M. H. et al., 2013, Nat Rev Cancer, 13(8):525-41). However, ACT is only able to cure a small proportion of the patients treated (Rosenberg, S. A. 2012, Sci Transl Med, 4(127):127ps8; Phan, G. Q. et al., 2013, Cancer Control, 20(4):289-97; Gajewski, T. F. et al., 2013, Curr Opin Immunol, 25(2):268-76; Gajewski, T. F. et al., 2013, Nat Immunol, 14(10):1014-22), leaving substantial room for improvement.

Thus, there is a need in the art for improved compositions and methods for identifying and generating improved therapeutic cells for adoptive T cell therapy. The present invention satisfies this unmet need.

### SUMMARY OF THE INVENTION

In various aspects, the present invention provides a method for identifying one or more T cells with enhanced features, comprising obtaining one or more T cells, and separating from the one or more T cells, one or more T cells possessing high Golgi mass (Golgi^{hi} T cells). In some embodiments, the separating comprises staining the one or more T cells with a dye that stains the Golgi apparatus and sorting the stained one or more cells via flow cytometry to obtain one or more cells in which the amount of dye within the one or more cells is above a predetermined threshold (Golgi^{hi} T cells); sorting the one or more T cells to obtain one or more T cells in which levels of branched N-glycans are below a predetermined threshold (Golgi^{hi} T cells); sorting the one or more T cells to obtain one or more T cells in which levels of MGAT1 (β1,6 N-acetylglucosaminyltransferase I) mRNA are above a predetermined threshold (Golgi^{hi} T cells); or sorting the one or more T cells to obtain one or more T cells in which levels of MGAT5A/B (β1,6 N-acetylglucosaminyltransferase Va/Vb) mRNA are below a predetermined threshold (Golgi^{hi} T cells). In some embodiments, the dye is Bopidy™ TR Ceramide. In some embodiments, the flow cytometry is Fluorescence Activated Cell Sorting (FACS).

In various aspects, the present invention provides a method for generating one or more T cells with enhanced features, comprising obtaining one or more T cells, and performing at least one of the following steps: administering to the one or more T cells to an agent that releases H₂S in the one or more T cells; administering to the one or more T cells to an agent that over-expresses cystathione β-synthase (CBS) in the one or more T cells; administering to the one or more T cells to an agent that inhibits MGAT5 enzymatic activity, or a combination thereof. In some embodiments, the agent that over-expresses CBS in the one or more T cells is selected from the group consisting of a plasmid, a virus, a nucleic acid molecule, and any combination thereof. In some embodiments, the agent that releases H₂S is selected from the group consisting of GYY 4137 and sodium hydrogen sulfide (NaHS). In some embodiments, the agent that inhibits MGAT5 enzymatic activity comprises Phostine PST3.1a.

In some embodiments, the one or more T cells is selected from the group consisting of one or more CD4+ T cells, one or more CD8+ T cells, one or more tumor infiltrating lymphocytes (TILs), one or more memory T cells (T_{CM}), and any combination thereof. In some embodiments, the one or more T cells is isolated from a subject. In some embodiments, the one or more T cells are engineered to express a chimeric antigen receptor (CAR). In some embodiments,

In some embodiments, enhanced features comprise increased protein translation, resistance to T cell exhaustion, increased production of proinflammatory cytokines, increased mitochondrial mass, increased mitochondrial function, increased spare respiratory capacity, upregulation of metabolic pathways, and anti-tumor activity. In some embodiments, the metabolic pathways comprise glutathione metabolism, nicotinate/nicotinamide metabolism, and the mitochondrial electron transport. In some embodiments, the anti-tumor activity comprises overall tumor volume reduction, increased survival and engraftment of the one or more T cells following transplant, and superior induction of tumor cell death.

In various aspects, the present invention provides a composition comprising the one of more T cells with enhanced features. In various aspects, the present invention provides a method of treating cancer in a subject in need thereof, the method comprising administering to the subject the composition of the present invention.

In various aspects, the present invention provides a method of increasing the presence of Golgi^{hi} T cells in a population of T cells, the method comprising administering to the population of T cells at least one of the following: an agent that releases H₂S in one or more T cells of the population of T cells; an agent that over-expresses cystathione β-synthase (CBS) in the one or more T cells in one or more T cells of the population of T cells; and an agent that inhibits MGAT5 enzymatic activity in one or more T cells of the population of T cells.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts a representative image of the process of adoptive cell therapy (ACT). T cells are isolated from either tumor (TIL) or blood (PBMC, peripheral blood mononuclear cells). Cells are transduced with genes encoding a chimeric antigen receptor (CAR) or T cell receptor (TCR)

FIG. 2 presents a representative image depicting the causes of immune cell dysfunction in the tumor microenvironment (TME).

FIG. 3 presents a representative image depicting hydrogen sulfide (H₂S) as a cryoprotective signaling molecule.

FIG. 4 depicts representative data demonstrating that H₂S supported T cell effector function and protein translation.

FIG. 5 depicts representative data demonstrating that H₂S enhanced the antitumor efficacy of T cells in vivo.

FIG. 6A through FIG. 6D depict representative data demonstrating that H₂S reduced oxidative stress and stress within the Golgi-ER axis in antitumor T cells.

FIG. 7 depicts representative data demonstrating that oxidative stress induced Golgi stress which was mitigated with H₂S.

FIG. 8 depicts representative data demonstrating that H₂S was partially dependent on the antioxidant function of Prdxz4.

FIG. 9A through FIG. 9O depict representative data demonstrating that high mass Golgi (Golgi^{hi}) T cells displayed enhanced functionality and stem-like features. (FIG. 9A through FIG. 9K) Pmel CD8+ T cells were activated for 3 days with gp100 and IL2+/−0.5 mM GYY4137. On day 3, cells were stained with BODIPY TR Ceramide dye to label Golgi apparatus. Labeled cells were sorted into Golgi^{hi} (upper 25%) and Golgi^{lo} (lower 25%) based on BODIPY TR ceramide dye fluorescence using FACS Aria III cell sorter. (FIG. 9B) Sorted cells were re-stimulated with gp100 peptide (1 μg/mL) and analyzed for protein translation using Click-iT Plus OPP Alexa Fluor 647 Protein Synthesis Assay Kit. Shown is quantification of MFI (n=4 independent samples). (FIG. 9C) FACS quantification of central memory (CD62L+CD44+ double positive) populations in Golgi^{lo} versus Golgi^{hi} cells (n=4 independent experiments). FACS plots are representative of 4 independent samples. (FIG. 9D) Quantification of FACS MFI values for Sca-1 and CD27 expression in Golgi^{lo} versus Golgi^{hi} cells (n=4 independent samples). (FIG. 9E) Golgi^{lo} versus Golgi^{hi} cells were subjected to in vitro exhaustion assays and analyzed via FACS for expression of TIM3. Shown is the quantification of TIM3+ cells in Golgi^{lo} versus Golgi^{hi} subset (n=5 independent samples). (FIG. 9F) Golgi^{lo} versus Golgi^{hi} cells were re-stimulated with gp100 peptide (1 ug/mL) and the supernatant was collected for multiplex ELISA cytokine analysis. Shown is a heat map illustrating the log 10 (Fluorescence Intensity) of various cytokines for Golgi^{lo} versus Golgi^{hi} groups (n=3 independent samples). (FIG. 9G) Quantification of FACS MFI values for MitoTracker Deep Red FM stain in Golgi^{lo} versus Golgi^{hi} cells (n=4 independent samples). (FIG. 9H) Real-time flux in OCR measured via Seahorse metabolic flux analysis. Shown is the quantification of mean oxygen consumption rate (OCR) throughout the assay, spare respiratory capacity (SRC), maximal respiration, and basal respiration (n=experimental replicates representative of 3 separate experiments). (FIG. 9I) Golgi^{lo} versus Golgi^{hi} Pmel T cells were analyzed via transmission electron microscopy (TEM) to evaluate mitochondria and golgi morphology; Golgi^{hi} cells possess enhanced mitochondrial function. (FIG. 9J through FIG. 1) Global metabolomics analysis was performed on Golgi^{lo} versus Golgi^{hi} Pmel T cells and analyzed for PCA (FIG. 9J) and pathway enrichment (FIG. 9K); metabolomics analysis revealed significant differences in the metabolite profiles of Golgi^{hi} versus Golgi^{lo} cells (FIG. 9J). (FIG. 9L) levels of Nad+ and NADH obtained from metabolomics. (FIG. 9M through FIG. 9O) Sorted cells were adoptively transferred into B16F10-bearing mice. (FIG. 9M) Tumor size measurements and overall survival for B16F10 tumor-bearing C57BL/6 mice treated with sorted Pmel T cells (n=5 mice per group). n, At day 14 after transfer, the mice were bled to analyze the frequency of circulating Pmel T cells. (FIG. 9O) Human CD19 CAR-T cells were expanded and sorted into Golgi^{hi} and Golgi^{lo} based on BODIPY TR Ceramide dye fluorescence and were co-cultured at a 1:1 ratio with Raji cells. The percentage of viable Raji cells remaining after 24 hours was analyzed via FACS. All data shown represent the mean±SEM and were analyzed by two-sided Student's t-test or oneway ANOVA unless otherwise specified. ns, P>0.05; *, P≤0.05; **, P≤0.01; ***, P≤0.001; ****, P≤0.0001

FIG. 10A through FIG. 10C depict representative data demonstrating that Golgi^{hi} T cells exerted superior tumor control.

FIG. 11 presents a representative image depicting the T cell exhaustion in the TME (left panel) and the protective role of H₂S (right panel). Exhausted T cells experience Golgi stress and disruption of Golgi function. H₂S signaling reduces Golgi stress by involving Prdx4. Tumor epitope reactive TCR bearing or CD19 CAR-engineered T cells treated with H₂S display enhanced stemness, mitochondrial function, antioxidant capacity, and superior tumor control. Tumor epitope reactive TCR bearing or CD19 CAR-engineered T cells with higher Golgi content possess a unique metabolic signature and display superior tumor control.

FIG. 12A through FIG. 12L depict representative data demonstrating that H₂S promotes generation of central memory (Tcm) anti-tumor T cells. (FIG. 12A) splenocytes from Pmel mice were stained with CTV dye and stimulated with gp100 antigen (1 ug/mL)+IL2 (100 IU/mL) followed by staining with WSP-1 dye to quantify H₂S production using FACS (n=4 independent samples). (FIG. 12B) RT-PCR analysis performed on Pmel CD8+ T cells at various timepoints following activation to quantify mRNA levels of CBS (n=4). FIG. 12C-12E, Pmel T cells treated with 0.5 mM GYY4137 for 3 days. (FIG. 12C) FACS analysis for expression of CD62L and CD44 (n=4). (FIG. 12D) FACS analysis for expression of Tcf1/7 and Sca-1 (n=4). FIG. 12E-12F, WT and Cbs-KO splenocytes activated with anti-CD3 and anti-CD28 and expanded with addition of H₂S to the Cbs-KO cells. (FIG. 12E) Analysis of intracellular H₂S by FACS using WSP-1 dye and (FIG. 12F) FACS analysis of relative percentage of CM to EM cells (n=3). (FIGS. 12G and 12H) Activated Pmel T cells transduced with lentiviral vector to overexpress Cbs enzyme (Cbstd) and on day 7 analyzed for intracellular H₂S production using WSP-1 dye (FIG. 12G), and frequency of CM (CD62L+CD44+) and Sca-1 expression (n=4) (FIG. 12H). (FIG. 12I-12L) B16-F10 tumors implanted in C57BL/6 mice and resected on day 14 to culture in vitro. Supernatant from the cultured tumor cells was extracted and transferred to cultures containing Pmel T cells and gp100 antigen+/−0.5 mM GYY4137. T cells cultured under the conditions of the exhaustion assay were analyzed for expression of Cbs by FACS (FIG. 12J), and intracellular H₂S using WSP-1 dye and quantified by FACS (n=4) (FIG. 12K). (FIG. 12L) T cells from tumor supernatant exhaustion assay were also analyzed for the expression of CD62L and CD44 via FACS (n=4). All data shown represent the mean±SEM and were analyzed by two-sided Student's t-test or one-way ANOVA. ns, p-value >0.05; p-value *≤0.05; **≤0.01; ***≤0.001; ****≤0.0001.

FIG. 13A through FIG. 13K depict representative data demonstrating that H₂S promotes generation of central memory (Tcm) anti-tumor T cells. (FIG. 13A) Pmel T cells were stained with trypan blue stain on day 3 following activation with gp100+IL2 (100 IU/ml) and treated with concentrations of GYY4137 (n=5). (FIG. 13B) Gating strategy for FACS analysis. Pmel splenocytes stained with LIVE/DEAD Fixable Yellow dye and anti-CD8 PE-Cy7. All FACS samples were first gated on the lymphocyte population based on FSA vs SSA. Single cells were gated using FSA vs. FSH. Live cells were gated using live/dead exclusion dye. CD8+ cells were gated by selecting the positive population. (FIG. 13C) Pmel T cells were stimulated with gp100 tumor antigen and treated with either vehicle or GYY4137 (0.5 mM) and analyzed by FACS for expression of CD62L and CD44 at day 0, day 2, day 4, and day 6. (FIG. 13D) Pmel T cells activated in the presence of various concentrations of NaHS and analyzed for CD62L and CD44 expression by FACS. (FIG. 13E) Pmel T cells activated with gp100 for 3 days and subsequently sorted by FACS to obtain effector memory (EM, CD62L-CD44+) and central memory (CM, CD62L+CD44+) populations. mRNA was isolated from each sorted population and RT-PCR was performed to determine mRNA levels of CSE, CBS, and 3-MST (n=4). (FIG. 13F) CD8+ Pmel T cells were activated for 3 days in the presence of various cytokines and analyzed for mRNA expression of CBS and CSE. (FIG. 13G) Splenocytes were isolated from wildtype and Cbs-knockout mice. Splenocytes were activated with anti-CD3 and anti-CD28 antibodies an analyzed for expression of Cbs by western blot. (FIG. 13H) Splenocytes from WT and Cbs-KO mice were expanded with either IL-2 or expanded with IL-15 (10 ng/ml) with or without addition of H₂S to the Cbs-KO cells and analyzed for expression of CD62L and CD44 by FACS (n=3). (FIG. 13I) T cells obtained from WT and Cbs-KO spleens were activated and expanded prior to adoptive transfer into Rag−/− mice (1×106 cells per mouse). At day 14, the mice were bled and the frequency of CD3+ T cells was assessed by FACS (n=5). (FIG. 13J) Pmel T cells were transduced with either control or CBS lentivirus and transduction efficiency was analyzed by assessing GFP expression by FACS. (FIG. 13K) Cells were analyzed using FACS for expression of PD1, TIM3, Lag3, and CD38 (n=3). MFI represents mean fluorescence intensity. All data shown represent the mean #SEM and were analyzed by two-sided Student's t test or one-way ANOVA. ns, p value >0.05; p value *≤0.05; **≤0.01; ***≤0.001; ****≤0.0001.

FIG. 14A through FIG. 14I depict representative data demonstrating that H₂S supports T cell effector function and protein translation. (FIG. 14A-14D) Melanoma epitope gp100 TCR reactive CD8+ T cells were activated for 3 days with gp100 and IL2+/−0.5 mM GYY4137. On day 3, the cells were collected for RNA-sequencing analysis (n=3 independent samples). (FIG. 14A) Principle component analysis (PCA) of control versus H₂S-treated samples. (FIG. 14B) Heat map displaying significantly upregulated and downregulated gene groups. FIG. 14C, Volcano plot demonstrating significantly upregulated and downregulated genes using false discovery rate (FDR)-adjusted P value <0.05. (FIG. 14D) Pathway enrichment analysis of significantly upregulated genes. (FIG. 14E) Schematic of experimental design for cytokine and translation assays. (FIG. 14F) Pmel CD8+ T cells were activated for 3 days with gp100 and IL2+/−0.5 mM GYY4137. On day 3, the cells were washed and re-stimulated with gp100 for 4 hours, followed by measurement of cytokine production via FACS. (FIG. 14G) Active protein translation was assessed using Click-iT Plus OPP Alexa Fluor 647 Protein Synthesis Assay Kit (ThermoFisher) (n=3 independent samples) in Pmel T cells after re-stimulation (n=3 independent samples). (FIG. 14H) Levels of EIF2a, phosphorylated 4EBP1, and phosphorylated rS6 evaluated via FACS in Pmel T cells after re-stimulation (n=4 independent samples). (FIG. 14I) Pmel CD8+ T cells were activated for 2 days with gp100 and IL2 followed by transduction with either control lentivirus or CBS-expressing lentivirus. On day 5, the cells were washed and re-stimulated with gp100 for 4 hours. Quantification of protein translation assessed using Click-iT Plus OPP Alexa Fluor 647 Protein Synthesis Assay Kit and quantification intracellular cytokines assessed using FACS (n=3 independent samples). MFI represents mean fluorescence intensity. All data shown represent the mean±SEM and were analyzed by two-sided Student's t-test or one-way ANOVA, unless otherwise specified. ns, p value >0.05; p value *≤0.05; **≤0.01; ***≤0.001; ****≤0.0001.

FIG. 15A and FIG. 15B depict representative data demonstrating that H₂S supports T cell effector function and protein translation. (FIG. 15A) Significantly upregulated genes identified 1053 in RNA-seq analysis. (FIG. 15B) Melanoma epitope gp100 TCR reactive CD8+ T cells were activated for 3 days with gp100 and IL2+/−0.5 mM GYY4137. On day 3, the cells were collected and TET activity was assessed using nuclear protein extract from CD8+ T cells treated with or without 0.5 mM GYY4137. Data shown represent the mean±SEM and were analyzed by two-sided Student's t-test or one-way ANOVA, unless otherwise specified. Ns, p value >0.05; p value *≤0.05; **≤0.01; ***≤0.001; ****≤0.0001.

FIG. 16A through FIG. 16D depict representative data demonstrating that H₂S enhances the antitumor efficacy of T cells in vivo. (FIG. 16A-16D) Indicated cells were expanded ex vivo and transferred into mice bearing subcutaneously inoculated tumors. Tumor size was measured 3 times per week until endpoint size of 400 mm2. All tumor control experiments were repeated twice. (FIG. 16A) B16-F10 melanoma tumor-bearing C57BL/6 mice treated with melanoma epitope gp100 TCR reactive CD8+ T cells cultured with IL-2 alone or IL-2+0.5 mM GYY4137 (n=10 mice per group). Frequency of Pmel T cells in peripheral blood at day 21 post-transfer assessed by FACS. (FIG. 16B) B16F10 tumor-bearing C57BL/6 mice treated with TILs isolated from B16-F10 tumors grown subcutaneously on C57BL/6 mice and expanded with IL-2 alone or IL-2+0.5 mM GYY4137 (n=10 mice per group). Frequency of transferred T cells in peripheral blood at day 10 post-transfer assessed by FACS. (FIG. 16C) Raji tumor-bearing NSG mice treated with human CD19 CAR-T cells cultured with IL-2 alone or IL-2+0.5 mM GYY4137 (n=10 mice per group). CAR-T cells were injected 3 days following Raji cell inoculation. Frequency of CD34+ transferred CAR-T cells in peripheral blood at day 21 post-transfer assessed by FACS. (FIG. 16D) Human PBMCs were transduced with either CD34-CD19-CAR-T construct or Cbs-CD34-CD19-CAR-T construct. Transduced CAR-T cells were sorted on CD34+ cells and adoptively transferred into CD19+ Raji tumor-bearing NSG mice 6 days after Raji cell inoculation (n=10 mice per group). Frequency of CD34+ transferred CAR-T cells in peripheral blood at day 21 post-transfer assessed by FACS. For all survival outcomes, Kaplan-Meier curves were used to display the results. Median survival time and corresponding 95% confidence interval were calculated for each experimental condition. A log-rank test was used to compare the outcomes across experimental conditions. ns, p value >0.05; p value *≤0.05; **≤0.01; ***≤0.001; ****≤0.0001.

FIG. 17A through FIG. 17D depict representative illustration depicting the general schema for experiments using Pmel T cells with B16F10 melanoma tumors (FIG. 17A), TILs isolated from B16F10 melanoma tumors and used to treat B16F10 tumors in Rag−/− mice (FIG. 17C), or generation of human CD19-CAR-T cells to treat huma Raji lymphoma tumors in NSG mice (FIG. 17D). (FIG. 17B) At day 21 following transfer of Pmel T cells into mice bearing B16F10 melanoma tumors, mice were bled and the retrieved cells were analyzed for expression of memory and stemness markers by FACS. All data shown represent the mean±SEM and were analyzed by two-sided Student's t-test or one-way ANOVA. ns, p value >0.05; p value *≤0.05; **≤0.01; ***≤0.001; ****≤0.0001.

FIG. 18A through FIG. 18K depict representative data demonstrating that H₂S alters metabolic profile and enhances mitochondrial function of T cells. (FIG. 18A-18C) Pmel T cells activated for 3 days with gp100 and IL2+/−0.5 mM GYY4137 and collected for comprehensive metabolomics analysis. a, Pathway enrichment analysis based on significantly different metabolites. (FIG. 18B) Volcano plot showing significantly upregulated and downregulated metabolites based on −log 10(p)>1. (FIG. 18C) NAD+ and nicotinamide quantification from metabolomics dataset. (FIG. 18D) SIRT1 deacetylase activity in activated Pmel T cells (means of normalized OD values). (FIGS. 18E and 18F) Pmel T cells were activated with gp100 for 3 days in the presence of vehicle, GYY4137 (0.5 mM), or GYY4137+sirt1 inhibitor EX527 (10 μM). (FIG. 18E) Nuclear and cytoplasmic protein fractions were isolated and western blot analysis was performed to determine relative expression of nuclear and cytoplasmic Foxo1 (n=3). (FIG. 18F) Expression of CD62L and CD44 analyzed by FACS (n=3). (FIG. 18G) Splenocytes from wildtype and Cbs-knockout mice+/−GYY4137 activated with anti-CD3 and anti-CD28 antibodies followed by intranuclear staining for Foxo1. (FIG. 18H) Pmel T cells activated and expanded for metabolic flux analysis. Quantification of oxygen consumption rate (OCR), spare respiratory capacity (SRC), maximal respiration, and basal respiration (n=10 experimental replicates representative of 3 separate experiments). (FIG. 18I) Pmel T cells activated and expanded to day 5 for characterization of mitochondria using FACS, quantified by MFI values for mitoFM Deep Red, TMRM, and mitoSOX dyes (n=4). (FIG. 18J) Representative confocal microscopy images of mitochondria from control or H₂S-treated T cells. (FIG. 18K) Human CD19 CAR-T cells expanded for 3 days with IL2+/−0.5 mM GYY4137 and collected for metabolic flux analysis. Shown is the quantification of mean oxygen consumption rate (OCR), spare respiratory capacity (SRC), maximal respiration, and basal respiration. Data shown represent the mean±SEM and were analyzed by two-sided Student's t-test or one-way ANOVA. ns, p-value >0.05; p-value *≤0.05; **≤0.01; ***≤0.001; ****≤0.0001.

FIG. 19 depicts representative data demonstrating significantly upregulated and downregulated metabolites identified in metabolomics screen comparing vehicle versus H₂S-treated T cells.

FIG. 20A through FIG. 20O depict representative data demonstrating that H₂S reduces oxidative stress and Golgi-ER network stress in antitumor T cells. (FIG. 20A-20F) Pmel T cells activated for 3 days+/−0.5 mM GYY4137. (FIG. 20A) Cells treated for 12-hours with H₂O₂ (50 mM). FACS analysis quantifying 7-AAD+AnnexinV+ and 7−AAD−AnnexinV− populations (n=3). FIG. 20B, mRNA levels of GCLM and GCLC. (FIG. 20C) Measurements of surface thiols by AlexaFluor-488 C5 Maleimide (n=3) and intracellular glutathione by ThiolTracker Violet (n=4). (FIG. 20D) Proteomics analysis quantifying thiol modifications on cysteine residues with statistically significant results based on one sample T-test with FDR<0.5 with median of 3 normalized ratios using log 2 (FoldChange Treated vs. Control) (n=3). (FIG. 20E) Fold increase in MFI for phosphorylated-PERK, phosphorylated-IRE1a, and ATF4 after 12-hr. incubation with 50 mM H₂O₂ (n=3). FIG. 20F, FACS quantification of cells expressing high phosphorylated-PERK levels after exhaustion assay (n=2). (FIG. 20G) Percentage of cells expressing high phosphorylated-PERK in Pmel cells overexpressing Cbs compared to Pmel cells transduced with control lentivirus (n=3). (FIG. 20H) Fold change in GM130 MFI in Pmel cells using exhaustion assay (n=3). (FIG. 20I) Cell viability and mitochondrial ROS following monensin treatment (1 mM). (FIG. 20J) MFI values for GM130 and PD1 in TILs isolated from B16-F10 tumors and expanded+/−GYY4137 (n=3). (FIG. 20K) GM130 MFI values in Pmel T cells overexpressing Cbs compared to control cells after transfer into tumor-bearing mice and TIL isolation after 7 days (n=4). (FIG. 20L) Pmel T cells cultured with B16-F10 supernatant and gp100, followed by FACS sorting for PD1+TIM3+ and PD1−TIM3− cells. Sorted cells stained with anti-Giantin antibody and analyzed for Golgi area via ImageStream. (FIG. 20M) FACS analysis of GM130 expression in PD1+TIM3+ and PD1−TIM3− populations. (FIG. 20N) Pmel cells treated with 1 mM monensin and analyzed for intracellular ROS (DCFDA). (FIG. 20O) FACS analysis of GM130 expression in Pmel cells treated with 50 mM H₂O₂ (6-hours). Data represent mean±SEM. Analyzed by two-sided Student's t-test or one-way ANOVA. ns, p-value >0.05; p-value *≤0.05; **≤0.01; ***≤0.001; ****≤0.0001.

FIG. 21A through FIG. 21E depict representative data demonstrating that H₂S reduces oxidative stress and Golgi-ER network stress in antitumor T cells. (FIG. 21A) Levels of intracellular glutathione assessed using FACS by employing ThiolTracker Violet dye in control CD19 CAR-T cells or CAR-T cells overexpressing Cbs (CbstdCD19-CAR-T) (n=3). (FIG. 21B) 12 hour Thapsigargin (700 nM) treatment was used to induce ER stress in WT T cells, Cbs-KO T cells, and Cbs-KO T cells+H₂S. Levels of phosphorylated PERK were assessed by FACS (n=3). (FIG. 21C) Pmel T cells were treated with either Thapsigargin (700 nM) or Monensin (1 mM) for 12 hours, followed by staining for phosphorylated PERK (p-PERK) and GM130 to assess ER stress and Golgi stress, respectively (n=4). (FIG. 21D) Activated Pmel T cells were treated for 12 hours with either vehicle control or 1 mM Monensin (“+Golgi stress”) before being stained for giantin to assess Golgi dispersion. Image shows cells stained with anti-Giantin antibody and analyzed for Golgi dispersion (Golgi area) using ImageStream. (FIG. 21E) Representative confocal microscopy images of mitochondria (Tom20) and Golgi (giantin) in human PBMCs treated with vehicle control or 0.5 mM GYY4137. All data shown represent the mean±SEM and were analyzed by two-sided Student's t-test or one-way ANOVA unless otherwise specified. ns, p value >0.05; p value *≤0.05; **≤ 0.01; ***≤0.001; ****≤0.0001.

FIG. 22A through FIG. 22K depict representative data demonstrating that Prdx4 regulates H₂S-mediated inhibition of Golgi stress. (FIG. 22A) Pmel T cells were activated for 3 days before treatment with vehicle or 50 mM H₂O₂ for 6 hrs. Prdx4-Golgi colocalization was assessed by PLA using mouse anti-Giantin and rabbit anti-Prdx4 antibodies. (FIG. 22B) Activated Pmel T cells treated with vehicle or 50 mM H₂O₂ for 6 hrs or 24 hrs with Monensin (1 mM). PLA assay was performed using anti-Giantin and anti-Prdx4 antibodies. (FIG. 22C) Experimental design using Prdx4-targeted siRNA to silence expression of Prdx4 in Pmel T cells. (FIG. 22D) GM130 expression assessed under control versus Monensin (1 μM) conditions in T cells treated with control-siRNA or Prdx4-siRNA. (FIG. 22E) GM130 expression assessed under control versus 50 mM H₂O₂ conditions in T cells treated with control-siRNA or Prdx4-siRNA+/−0.5 mM GYY4137. (FIG. 22F) Protein translation assessed in Pmel T cells activated+/−GYY4137 and treated with control-siRNA or Prdx4-siRNA. (FIG. 22G) GM130 expression in Pmel T cells treated with vehicle, GYY4137, or Catalase (10 μg/mL). (FIGS. 22H and 22I) Site directed mutagenesis performed to mutate cysteine-54 of the Prdx4 gene. Prdx4-targeted shRNA was used to knock down expression of Prdx4 in Jurkat T cells, followed by transfection with plasmid containing wildtype or mutated Prdx4. (FIG. 22I) GM130 expression assessed after treatment with 50 mM H₂O₂+/−GYY4137. (FIG. 22J) B16-F10 tumors were injected s.c. into flanks of Pmel mice and grown to ˜100 mm2. TILs and T cells from tumor-draining lymph nodes were isolated via Ficoll spin followed by magnetic bead positive selection. FACS analysis of Prdx4 expression in TILs gated on PD1loLag3lo vs PD1hiLag3hi. (FIG. 22K) Lentiviral supernatant was generated using GFP-tagged control plasmid or GFP-tagged Prdx4-overexpression plasmid. Pmel T cells were transduced with control or Prdx4 lentivirus. Pmel T cells overexpressing Prdx4 and control T cells were treated with H₂O₂ (50 mM) followed by staining with anti-GM130. Data shown represent mean±SEM and analyzed by two-sided Student's t-test or one-way ANOVA. ns, p-value >0.05; p-value *≤0.05; **≤0.01; ***≤0.001; ****≤0.0001.

FIG. 23A through FIG. 23G depict representative data demonstrating that Prdx4 regulates H₂S-mediated inhibition of Golgi stress. (FIG. 23A) Transfection efficiency of control siRNA (GFP−) and siRNA directed against Prdx4 (GFP+) assessed via expression of GFP. (FIG. 23B) Expression of Prdx4 assessed by western blot. (FIG. 23C) FACS analysis of cytokine production by Pmel T cells transfected with either control siRNA (siCtl) or siRNA directed against Prdx4 (siPrdx4) (n=4). (FIG. 23D) Expression of phosphorylated-PERK in Prdx4-knockout Jurkat cells transfected with either wildtype Prdx4 plasmid or mutant Prdx4 plasmid (n=2). (FIG. 23E) B16-F10 tumors were injected s.c. into the flanks of Pmel mice. Upon reaching tumor volume of approximately 100 mm2, tumor infiltrating lymphocytes (TILs) and T cells from tumor draining lymph nodes (LN) were isolated via Ficoll gradient spin followed by magnetic bead positive selection. FACS analysis of Prdx4 expression in PD1+ TILs vs LN T cells (n=4). (FIGS. 23F and 23G) Lentiviral supernatant was generated using either GFP-tagged control plasmid or GFP-tagged Prdx4-overexpression plasmid. Activated Pmel T cells were transduced with either control or Prdx4 lentivirus for subsequent assays. (FIG. 23F) Overexpression of Prdx4 was confirmed by FACS. (FIG. 23G) Pmel T cells overexpressing Prdx4 and control T cells were restimulated with gp100 and analyzed for cytolytic cytokine production by FACS. All data shown represent the mean±SEM and were analyzed by two-sided Student's t-test or one-way ANOVA unless otherwise specified. ns, p value >0.05; p value *≤0.05; **≤0.01; ***≤0.001; ****≤0.0001.

FIG. 24A through FIG. 24O depict representative data demonstrating that Golgi^{hi} cells possess enhanced functionality and stem-like features. (FIG. 24A through 24K) Pmel T-cells were activated for 3 days+/−GYY4137, then stained with BODIPY-TR Ceramide dye to label Golgi, followed by FACS-sorting into Golgi^{hi} (upper 30%) and Golgi^{lo} (lower 30%) based on BODIPY-TR Ceramide dye fluorescence. (FIG. 24B) FACS quantification of GM130 and H₂S levels in Golgi^{hi} vs Golgi^{lo} cells. Sorted cells were re-stimulated (gp100 1 ug/mL) and analyzed for protein translation using Click-iT Plus Protein Synthesis Kit (n=4). (FIG. 24C) FACS quantification of CD62L+CD44+ populations in sorted cells (n=4). (FIG. 24D) FACS quantification of Sca-1 and CD27 in sorted cells (n=4). (FIG. 24E) FACS analysis of TIM3 expression in sorted cells after in vitro exhaustion assay (n=5). (FIG. 24F) Sorted cells were re-stimulated (gp100 1 ug/mL) and supernatant was collected for multiplex cytokine ELISA. Heat map illustrating log 10 (Fluorescence Intensity) of cytokines for Golgi^{lo} versus Golgi^{hi} groups (n=3). (FIGS. 24G and 24H) Overall survival for B16-F10 tumor-bearing C57BL/6 mice treated with FACS-sorted Pmel T-cells (n=10) (FIG. 24G), Raji tumor-bearing NSG mice treated with FACS-sorted CD19-CAR-T cells (n=10) (FIG. 24H). (FIG. 24I) Sorted Pmel T-cells were injected into B16F10-bearing mice. TILs isolated by Ficoll separation and CD8+ selection stimulated with gp100 followed by FACS analysis of TNFα and IFNγ (n=4). (FIG. 24J) qRT-PCR analysis of mRNA levels for glycosylation enzymes in Golgi^{hi} vs. Golgi^{lo} human CD8+ T-cells. (FIG. 24K) Golgi^{hi} vs. Golgi^{lo} human CD8+ T-cells analyzed using antibody-based platform with MALDI-IMS to assess N-linked glycosylation. (FIGS. 24L and 24M) Levels of branched N-glycans with individual branched N-glycans that were significantly different between groups. (FIG. 24N) ELISA quantification of IFNγ production after CD19-CAR-T cells were treated with vehicle or Phostine-PST3.1a (10 μM) and co-cultured with Raji cells (n=3). (FIG. 24O) Pmel T-cells treated with vehicle or Phostine-PST3.1a prior to re-stimulation for FACS cytokine analysis (n=4). Data shown represent mean±SEM, analyzed by two-sided Student's t-test or one-way ANOVA. ns, p-value >0.05; p-value *≤0.05; **≤0.01; ***≤0.001; ****≤0.0001.

FIG. 25A through FIG. 25P depict representative data demonstrating that Golgi^{hi} cells possess enhanced functionality and stem-like features. (FIG. 25A) Activated Pmel T cells were sorted based on Golgi^{hi} vs Golgi^{lo} and expanded in culture with either vehicle control or GYY4137 (0.5 mM) for 3 days. Cells were subsequently stimulated with gp100 antigen and analyzed for production of cytokines by FACS (n=4). (FIG. 25B) Quantification of FACS MFI values for MitoTracker Deep Red FM stain in Golgi^{hi} vs. Golgi^{lo} cells (n=4 independent samples). (FIG. 25C) Real-time flux in OCR measured using Seahorse metabolic flux analysis. Shown is the quantification of mean oxygen consumption rate (OCR) throughout the assay, spare respiratory capacity (SRC), maximal respiration, and basal respiration (n=experimental replicates representative of 3 separate experiments). (FIG. 25D) FACS sorted Golgi^{hi} vs. Golgi^{lo} melanoma epitope gp100 TCR reactive CD8+ T cells from Pmel mouse were analyzed using transmission electron microscopy (TEM) to evaluate mitochondria and Golgi morphology. (FIG. 25E-25H) Global metabolomics analysis was performed on Golgi^{hi} vs. Golgi^{lo} Pmel T cells and analyzed for PCA (FIG. 25E) and pathway enrichment (FIG. 25F). (FIG. 25G) Significantly upregulated and downregulated metabolites (Golgi^{hi} vs. Golgi^{lo}). (FIG. 25H) Levels of NAD+ and NADH obtained from metabolomics. (FIG. 25I) Tumor size measurements for C57BL/6 mice bearing B16-F10 tumors treated with FACS sorted T cells (n=10 mice per group). (FIG. 25K) At day 14 after transfer, the tumor-bearing mice were bled to analyze the frequency of circulating Pmel T cells and for expression of surface markers using FACS (FIG. 25L). (FIG. 25J) Tumor size measurements for Raji tumor-bearing NSG mice treated with FACS sorted CD19 CAR-T cells (n=10 mice per group). (FIGS. 25M and 25N) Human CD19 CAR-T cells were expanded and sorted into Golgi^{hi} and Golgi^{lo} based on BODIPY TR Ceramide dye fluorescence and were co-cultured at a 1:1 ratio with Raji cells. (FIG. 25M) The percentage of viable Raji cells remaining after 24 hours was analyzed using FACS. (FIG. 25N) Expression of Perforin and Granzyme B was assessed using FACS in sorted CD19 CAR-T cells following co-culture with Raji cells. (FIGS. 25O and 25P) bulk RNA-seq analysis performed on human CD19 CAR-T cells FACS sorted on Golgi^{hi} vs Golgi^{lo} (n=3 independent samples from 3 separate donors). (FIG. 25O) Heat map cluster analysis showing significantly up- and down-regulated genes. (FIG. 25P) Significantly upregulated pathways in the Golgi^{hi} subset identified by Gene Ontology (GO) enrichment analysis. All data shown represent the mean±SEM and were analyzed by two-sided Student's t-test or one-way ANOVA unless otherwise specified ns, p value >0.05; p value *≤0.05; **≤0.01; ***≤0.001; ****≤0.0001.

### DETAILED DESCRIPTION

The present invention relates to one or more T cells having enhanced features and is based, in part, upon the unexpected findings that T cells comprising high Golgi mass, T cells treated with H₂S, T cells modified to overexpress cystathione β-synthase (CBS), and T cells treated with an agent that inhibits MGAT5 enzymatic activity display enhanced features. Accordingly, in one embodiment, the present invention comprises methods and compositions relating to one or more T cells having enhanced features.

In one embodiment, the enhanced features comprise, without limitation, increased protein translation, resistance to T cell exhaustion, increased production of proinflammatory cytokines, increased mitochondrial mass, increased mitochondrial function, increased spare respiratory capacity, upregulation of metabolic pathways, and anti-tumor activity, relative to a non-Golgi^{hi} T cells, a T cell not treated with H₂S, a T cell not modified to overexpress CBS, or a T cell not treated with an agent that inhibits MGAT5 enzymatic activity, wherein the metabolic pathways comprise glutathione metabolism, nicotinate/nicotinamide metabolism, and the mitochondrial electron transport, and wherein the anti-tumor activity comprises overall tumor volume reduction, increased survival and engraftment of transplanted T cells of the present invention, and superior induction of tumor cell death.

In one embodiment, the invention comprises methods for identifying one or more T cells with enhanced features, wherein the one or more T cells comprises high Golgi mass (Golgi^{hi} T cells), wherein the method comprises obtaining one or more T cells, separating from the one or more T cells, one or more T cells possessing high Golgi mass (Golgi^{hi} T cells), wherein the separating comprises staining the one or more T cells with a dye that stains the Golgi apparatus; sorting the stained one or more cells via flow cytometry to obtain one or more cells in which the amount of dye within the one or more cells is above a predetermined threshold to obtain Golgi^{hi} T cells. In one embodiment, the dye is Bopidy™ TR Ceramide. In one embodiment, the flow cytometry is Fluorescence Activated Cell Sorting (FACS).

In one embodiment, the invention comprises methods for the generation of one or more T cells having enhanced features. In one embodiment, the method comprises obtaining one or more T cells, and administering to the one or more T cells to H₂S using an agent that releases H₂S in the one or more T cells. In one embodiment, the method comprises obtaining one or more T cells, and administering to the one or more T cells CBS or an agent that over-expresses CBS in the one or more T cells. In one embodiment, the method comprises obtaining one or more T cells, and administering to the one or more T cells catalase or an agent that over-expresses catalase in the one or more T cells. In one embodiment, the method comprises obtaining one or more T cells, and administering to the one or more T cells an agent that inhibits MGAT5 enzymatic activity.

In one embodiment, the invention comprises compositions relating to one or more T cells having enhanced features. In one embodiment, the composition comprises an enriched population of the one or more T cells having enhanced features.

In one embodiment, the present invention relates to methods and compositions for treating or preventing cancer in a subject. In one embodiment, the methods for treating or preventing cancer in a subject comprising administering one or more T cells of the present invention having enhanced features to the subject. In one embodiment the one or more T cells having enhanced features is further modified to express a chimeric antigen receptor (CAR), wherein the CAR binds to a cell surface antigen on at least one tumor cell in the body subject.

In one embodiment, the methods for treating or preventing cancer in a subject comprise obtaining one or more T cells from the subject, generating one or more T cells having enhanced features from the one or more T cells from the subject, and administering the one or more T cells having enhanced features to the subject. In one embodiment, the one or more T cells obtained from the subject comprises one or more one or more tumor infiltrating cells (TILs). In one embodiment, the method further comprises modifying the one or more T cells having enhanced features to express a CAR, wherein the CAR binds to a cell surface antigen on at least one tumor cell.

In one embodiment, the compositions for treating or preventing cancer in a subject comprises an enriched population of one or more T cells having enhanced features. In one embodiment, the enriched population one or more T cells having enhanced features is further modified to express a CAR, wherein the CAR binds to a cell surface antigen on at least one tumor cell.

In one embodiment, the present invention relates to methods and compositions for treating or preventing a viral infection in a subject, wherein the methods and compositions generate anti-viral T cells, and wherein the anti-viral T cells reduce a viral load when introduced into the subject.

In one embodiment, the present invention relates to methods of increasing the presence of Golgi^{hi} T cells in a population of T cells, the method comprising administering to the population of T cells at least one of the following: an agent that releases H₂S in one or more T cells of the population of T cells; CBS or an agent that over-expresses cystathione β-synthase (CBS) in the one or more T cells in one or more T cells of the population of T cells; and an agent that inhibits MGAT5 enzymatic activity in one or more T cells of the population of T cells.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, +10%, +5%, +1%, or +0.1% from the specified value, as such variations are appropriate to perform the disclosed methods.

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “cancer” as used herein is defined as disease characterized by the aberrant proliferation and/or growth of cells. Cancer cells can spread locally or through the bloodstream and lymphatic system to other parts of the body. Cancer as here herein includes both solid tumors and hematopoietic malignancies.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or CDNA.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The terms “treatment” and “therapeutic” refer to compositions for alleviating or preventing symptoms of a disease or disorder. The terms “treat” and “therapy” refer to methods of administering a treatment or therapeutic to a subject in need thereof, for example, a subject afflicted with a disease or disorder, or a subject who ultimately may acquire such a disease or disorder, in order to prevent, cure, delay, reduce the severity of, or ameliorate one or more signs or symptoms of the disease or disorder or recurring disease or disorder.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

### DESCRIPTION

In one aspect the present invention comprises methods and compositions relating to one or more T cells having enhanced features. In one embodiment, the one or more T cells having enhanced features comprises T cells with high Golgi mass (Golgi^{hi} T cells). In one embodiment, the one or more T cells having enhanced features comprises T cells administered hydrogen sulfide (H₂S). In one embodiment, the one or more T cells having enhanced features comprises T cells modified to overexpress CBS. In one embodiment, the one or more T cells having enhanced features comprises T cells modified to overexpress catalase. In one embodiment, the one or more T cells having enhanced features comprises T cells administered with an agent that inhibits MGAT enzymatic activity.

In one aspect the present invention comprises methods and compositions for the treatment of cancer in a subject in need thereof, the method comprising administering to the subject the one or more T cells having enhanced features, of the present invention. In one aspect the present invention comprises methods and compositions for the treatment of cancer in a subject in need thereof, the compositions comprising the one or more T cells having enhanced features, of the present invention.

Methods for Identifying and Generating T Cells with Enhanced Features

In one aspect the present invention comprises methods for identifying and generating of one or more T cells having enhanced features.

In one embodiment the one or more T cells comprises CD4+ T cells, CD4+ T cells, tumor infiltrating lymphocytes (TILs), central memory T cells (T_{CM}), and any combination thereof. In one embodiment, the one of more T cells is a TIL isolated from the tumor of subject with cancer.

In one embodiment, the enhanced features displayed by the one or more T cells with enhanced features, of the present invention, comprise without limitation, increased protein translation, resistance to T cell exhaustion, increased production of proinflammatory cytokines, increased mitochondrial mass, increased mitochondrial function, increased spare respiratory capacity, upregulation of metabolic pathways, and anti-tumor activity, relative to cells not subject to the methods of the present invention, wherein the metabolic pathways comprise glutathione metabolism, nicotinate/nicotinamide metabolism, and the mitochondrial electron transport, and wherein the anti-tumor activity comprises overall tumor volume reduction, increased survival and engraftment of the one or more T cells having enhanced features following transplantation, and superior induction of tumor cell death.

Identifying T Cells with Enhanced Features

In one aspect the present invention comprises methods for identifying one or more T cells having enhanced features.

### High Golgi Mass T Cells (Golgi^{hi} T Cells)

In one embodiment, the invention comprises methods for the identifying one or more T cells having enhanced features. In one embodiment, the method comprises obtaining one or more T cells, separating from the one or more T cells, one or more T cells possessing high Golgi mass (Golgi^{hi} T cells), wherein the separating comprises staining the one or more T cells with a dye that stains the Golgi apparatus; sorting the stained one or more cells via flow cytometry to obtain one or more cells in which the amount of dye within the one or more cells is above a predetermined threshold to obtain Golgi^{hi} T cells.

In one embodiment, the dye is Bopidy™ TR Ceramide. In one embodiment, the flow cytometry is Fluorescence Activated Cell Sorting (FACS). In one embodiment, measuring the Golgi mass of the one or more T cells comprises staining the one or more T cells with a dye, wherein the dye stains the Golgi apparatus of the one or more T cells, detecting the dye, quantitating the amount of dye present in the one or more T cells based upon the amount of dye detected, and determining whether the one or more T cells is a high Golgi mass T cell (Golgi^{hi} T cell) based on a predetermined threshold.

In one embodiment, the staining comprises incubating the cells in the presence of a dye, wherein the dye is Bodipy™ TR Ceramide. In one embodiment, the dye is any dye commonly known the art to uniquely stain the Golgi apparatus of mammalian cells. In one embodiment, the detecting comprises fluorescence microscopy.

In one embodiment, separating Golgi^{hi} T cells from the one or more T cells obtained comprises flow cytometry-based sorting of the one or more T cells after staining with Bodipy™ TR Ceramide. In one embodiment, the Golgi^{hi} T cells are sorted from the one or more T cells obtained based upon a predetermined threshold of Bodipy™ TR Ceramide levels. In one embodiment, the flow cytometry comprises fluorescence-activated cell sorting (FACS). In one embodiment, a population of T cells is assessed for Bodipy™ TR Ceramide signal levels and cells with high signals (upper 25% of cells) are determined to be Golgi^{hi} T cells, while cells with low signals (lower 25% of cells) are determined to be Golgi^{lo} T cells. Accordingly, in one embodiment, the predetermined threshold for sorting Golgi^{hi} T cells is the upper 25th percentile.

In one embodiment, the method comprises obtaining one or more T cells, sorting the one or more T cells to obtain one or more T cells in which levels of branched N-glycans are below a predetermined threshold (Golgi^{hi} T cells), sorting the one or more T cells to obtain one or more T cells in which levels of MGAT1 (β1,6 N-acetylglucosaminyltransferase I) mRNA are above a predetermined threshold (Golgi^{hi} T cells), or sorting the one or more T cells to obtain one or more T cells in which levels of MGAT5A/B (β1,6 N-acetylglucosaminyltransferase Va/Vb) mRNA are below a predetermined threshold (Golgi^{hi} T cells).

Generating T Cells with Enhanced Features

In one aspect the present invention comprises methods for generating one or more T cells having enhanced features.

### Hydrogen Sulfide (H₂S) Administered T Cells

In one embodiment, the method comprises obtaining one or more T cells, administering to the one or more T cells an agent that releases H₂S in the one or more T cells. In one embodiment, the agent that releases H₂S in the one or more T cells comprises GYY 4137 or sodium hydrogen sulfide (NaHS).

In one embodiment, GYY 4137 is used at a concentration of 0.1-0.2 mM. In one embodiment, GYY 4137 is used at a concentration of 0.2-0.3 mM. In one embodiment, GYY 4137 is used at a concentration of 0.3-0.4 mM. In one embodiment, GYY 4137 is used at a concentration of 0.4-0.5 mM. In one embodiment, GYY 4137 is used at a concentration of 0.5-0.6 mM. In one embodiment, GYY 4137 is used at a concentration of 0.6-0.7 mM. In one embodiment, GYY 4137 is used at a concentration 0.5 mM.

In one embodiment, the one or more T cells are administered GYY 4137 for 1-2 days. In one embodiment, the one or more T cells are administered GYY 4137 for 2-3 days. In one embodiment, the one or more T cells are administered GYY 4137 for 3-4 days. In one embodiment, the one or more T cells are administered GYY 4137 for 4-5 days. In one embodiment, the one or more T cells are administered GYY 4137 for 5-6 days. In one embodiment, the one or more T cells are administered GYY 4137 for 6-7 days. In one embodiment, the one or more T cells are administered GYY 4137 for 3 days.

In one embodiment, NaHS is used at a concentration of 10-15 μM. In one embodiment, NaHS is used at a concentration of 15-20 μM. In one embodiment, NaHS is used at a concentration of 20-25 μM. In one embodiment, NaHS is used at a concentration of 25-30 μM. In one embodiment, NaHS is used at a concentration of 30-35 μM. In one embodiment, NaHS is used at a concentration of 35-40 μM. In one embodiment, NaHS is used at a concentration of 40-45 μM. In one embodiment, NaHS is used at a concentration of 45-50 μM. In one embodiment, NaHS is used at a concentration of 55-60 μM. In one embodiment, NaHS is used at a concentration of 60-65 μM. In one embodiment, NaHS is used at a concentration of 65-70 μM. In one embodiment, NaHS is used at a concentration of 70-75 μM. In one embodiment, NaHS is used at a concentration of 75-80 μM. In one embodiment, NaHS is used at a concentration of 80-85 μM. In one embodiment, NaHS is used at a concentration of 85-90 μM. In one embodiment, NaHS is used at a concentration of 90-95 μM. In one embodiment, NaHS is used at a concentration of 95-100 μM. In one embodiment, NaHS is used at a concentration of 100-105 μM. In one embodiment, NaHS is used at a concentration of 105-110 μM. In one embodiment, NaHS is used at a concentration of 110-115 μM. In one embodiment, NaHS is used at a concentration of 115-120 μM. In one embodiment, NaHS is used at a concentration of 120-125 μM. In one embodiment, NaHS is used at a concentration of 25 μM. In one embodiment, NaHS is used at a concentration of 50 μM. In one embodiment, NaHS is used at a concentration of 100 μM.

In one embodiment, the one or more T cells are administered NaHS for 1-2 days. In one embodiment, the one or more T cells are administered NaHS for 2-3 days. In one embodiment, the one or more T cells are administered NaHS for 3-4 days. In one embodiment, the one or more T cells are administered NaHS for 4-5 days. In one embodiment, the one or more T cells are administered NaHS for 5-6 days. In one embodiment, the one or more T cells are administered NaHS for 6-7 days. In one embodiment, the one or more T cells are administered NaHS for 3 days.

H₂S is an endogenous signaling molecule in mammalian cells and is a member of the “gasotransmitter” class of biological molecules (together with nitric oxide [NO] and carbon monoxide [CO]); it shares many properties with these molecules and, in many biological systems, it works with them in a coordinated and cooperative manner. Low concentrations of H₂S can exert physiological, regulatory, or modulatory effects, and act as cytoprotective, antioxidant and anti-inflammatory agents.

### CBS Over-Expressing T Cells

In one embodiment, the method comprises obtaining one or more T cells, and modifying the one or more T cells to over-express CBS. In one embodiment, the modifying comprises administering to the one or more T cells an agent that over-expresses CBS in the one or more T cells. In one embodiment, the agent that over-expresses CBS in the one or more T cells comprises a plasmid vector, a virus, a nucleic acid molecule, and any combination thereof. In one embodiment, the agent that over-expresses CBS in the one or more T cells comprises any factor known in the art to over-express a genetic construct in vitro or in vivo. In one embodiment, the modifying comprises administering CBS protein to the one or more T cells, wherein the CBS protein is administered to the cells via any technique known in the art to deliver proteins directly into cells.

In one embodiment, the over-expression of CBS in the one or more T cells with enhanced features is relative to CBS levels in a T cell in which CBS is not over-expressed. In one embodiment, the level of CBS comprises mRNA levels or protein levels.

In one embodiment, the over-expression of CBS in the one or more T cells is for 1-2 days. In one embodiment, the over-expression of CBS in the one or more T cells is for 2-3 days. In one embodiment, the over-expression of CBS in the one or more T cells is for 3-4 days. In one embodiment, the over-expression of CBS in the one or more T cells is for 4-5 days. In one embodiment, the over-expression of CBS in the one or more T cells is for 5-6 days. In one embodiment, the over-expression of CBS in the one or more T cells is for 6-7 days. In one embodiment, the over-expression of CBS in the one or more T cells is for 7-8 days. In one embodiment, the over-expression of CBS in the one or more T cells is for 8-9 days. In one embodiment, the over-expression of CBS in the one or more T cells is for 7 days.

### Catalase Over-Expressing T Cells

In one embodiment, the method comprises obtaining one or more T cells, and modifying the one or more T cells to over-express catalase. In one embodiment, the modifying comprises administering to the one or more T cells an agent that over-expresses catalase in the one or more T cells. In one embodiment, the agent that over-expresses catalase in the one or more T cells comprises a plasmid vector, a virus, a nucleic acid molecule, and any combination thereof. In one embodiment, the agent that over-expresses catalase in the one or more T cells comprises any factor known in the art to over-express a genetic construct in vitro or in vivo. In one embodiment, the modifying comprises administering catalase protein to the one or more T cells, wherein the catalase protein is administered to the cells via any technique known in the art to deliver proteins directly into cells.

In one embodiment, the over-expression of catalase in the one or more T cells with enhanced features is relative to catalase levels in a T cell in which CBS is not over-expressed. In one embodiment, the level of catalase comprises mRNA levels or protein levels.

In one embodiment, the over-expression of catalase in the one or more T cells is for 1-2 days. In one embodiment, the over-expression of catalase in the one or more T cells is for 2-3 days. In one embodiment, the over-expression of catalase in the one or more T cells is for 3-4 days. In one embodiment, the over-expression of catalase in the one or more T cells is for 4-5 days. In one embodiment, the over-expression of catalase in the one or more T cells is for 5-6 days. In one embodiment, the over-expression of catalase in the one or more T cells is for 6-7 days. In one embodiment, the over-expression of catalase in the one or more T cells is for 7-8 days. In one embodiment, the over-expression of catalase in the one or more T cells is for 8-9 days.

### MGAT5 Inhibited T Cells

In one embodiment, the method comprises obtaining one or more T cells, administering to the one or more T cells an agent that inhibits the enzymatic activity of MGAT5. In one embodiment, the agent that inhibits the enzymatic activity of MGAT5 comprises Phostine-PST3.1a.

In one embodiment, Phostine-PST3.1a is used at a concentration of 5-6 μM. In one embodiment, Phostine-PST3.1a is used at a concentration of 6-7 μM. In one embodiment, Phostine-PST3.1a is used at a concentration of 7-8 μM. In one embodiment, Phostine-PST3.1a is used at a concentration of 8-9 μM. In one embodiment, Phostine-PST3.1a is used at a concentration of 9-10 μM. In one embodiment, Phostine-PST3.1a is used at a concentration of 10-11 μM. In one embodiment, Phostine-PST3.1a is used at a concentration of 11-12 μM. In one embodiment, Phostine-PST3.1a is used at a concentration of 12-13 μM. In one embodiment, Phostine-PST3.1a is used at a concentration of 13-14 μM.

In one embodiment, the one or more T cells are administered Phostine-PST3.1a for 1-2 days. In one embodiment, the one or more T cells are administered Phostine-PST3.1a for 2-3 days. In one embodiment, the one or more T cells are administered Phostine-PST3.1a for 3-4 days. In one embodiment, the one or more T cells are administered Phostine-PST3.1a for 4-5 days. In one embodiment, the one or more T cells are administered Phostine-PST3.1a for 5-6 days. In one embodiment, the one or more T cells are administered Phostine-PST3.1a for 6-7 days.

### CAR-T Cells

In one embodiment, the one or more T cells with enhanced features of the present invention are modified to express a chimeric antigen receptor (CAR), wherein the one or more T cells expressing a CAR comprises a CAR-T cell. In one embodiment, the one or more T cells expressing a CAR comprises a CAR-T cell

In one embodiment, the CAR comprises an antigen binding domain which is specific for at least one marker of at least one cancer cell. In some embodiments, once bound to the at least one cancer cell, the CAR-T cell facilitates the destruction of the at least one cancer cell (e.g., by phagocytosis, T cell-mediated cytotoxicity, etc.), thereby treating or preventing a disease or disorder (e.g., cancer, etc.) in the subject.

The term “chimeric antigen receptor” or “CAR,” as used herein, refers to an artificial cell receptor that is engineered to be expressed on an immune effector cell, such as an NK cell, a macrophage, a B cell, or a dendritic cell, and specifically bind an antigen on at least one cancer cell or at least one pathogen. CARs may be used as a therapy with adoptive cell transfer. Generally, immune cells of interest, are removed from a patient and modified so that they express the receptors specific to a particular form of antigen. In some embodiments, the CARs have specificity to at least one cancer cell or at least one pathogen. CARs may also comprise an intracellular activation domain, a transmembrane domain and an extracellular domain comprising an antigen binding region that specifically binds to at least one cancer cell or at least one pathogen.

In various embodiments, the CARs contemplated herein comprise an extracellular domain, a transmembrane domain, and an intracellular domain. The extracellular domain comprises a target-specific binding element otherwise referred to as an antigen binding domain. In some embodiments, the extracellular domain also comprises a hinge domain. In certain embodiments, the intracellular domain or otherwise the cytoplasmic domain comprises, a costimulatory signaling region and a zeta chain portion. The costimulatory signaling region refers to a portion of the CAR comprising the intracellular domain of a costimulatory molecule. Costimulatory molecules are cell surface molecules other than antigens receptors or their ligands that are required for an efficient response of lymphocytes to antigen.

In various embodiments, the CAR can be a “first generation,” “second generation,” “third generation,” “fourth generation” or “fifth generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)), each of which are incorporated by reference in its entirety).

“First generation” CARs for use in the invention comprise an antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD32-chain, which is the primary transmitter of signals from endogenous T cell receptors (TCRs). “First generation” CARs can provide de novo antigen recognition and cause activation of both CD4+ and CD8+ T cells through their CD35 chain signaling domain in a single fusion molecule, independent of HLA-mediated antigen presentation.

“Second-generation” CARs for use in the invention comprise an antigen binding domain, for example, a single-chain variable fragment (scFv), fused to an intracellular signaling domain capable of activating T cells and a co-stimulatory domain designed to augment T cell potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD3ζ signaling domain. Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1(9):1577-1583 (2012)).

“Third generation” CARs provide multiple co-stimulation, for example, by comprising both CD28 and 4-1BB domains, and activation, for example, by comprising a CD3ζ activation domain.

“Fourth generation” CARs provide co-stimulation, for example, by CD28 or 4-1BB domains, and activation, for example, by a CD35 signaling domain in addition to a constitutive or inducible chemokine component.

“Fifth generation” CARs provide co-stimulation, for example, by CD28 or 4-1BB domains, and activation, for example, by a CD33 signaling domain, a constitutive or inducible chemokine component, and an intracellular domain of a cytokine receptor, for example, IL-2RB.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

In the embodiments disclosed herein, the CARs generally comprise an antigen binding domain, a transmembrane domain and an intracellular domain, as described above. In a particular non-limiting embodiment, the antigen-binding domain is an scFv.

In one embodiment, the antigen binding domain of the CAR molecule is a targeting domain, wherein the targeting domain directs the cell expressing the CAR to at least one cancer cell or at least one pathogen For example, in one embodiment, the targeting domain comprises an antibody, antibody fragment, or peptide that specifically binds to an antigen (e.g., a self-antigen or a foreign antigen) thereby directing the cell expressing the CAR to at least one cancer cell or at least one pathogen, wherein the at least one cancer cell or at least one pathogen expresses the antigen.

In one embodiment, the antigen binding domain of the CAR molecule of the invention can be generated to be reactive to any desirable antigen of interest, or fragment thereof, including, but not limited to a tumor antigen, a foreign antigen (e.g, a bacterial antigen, a viral antigen, etc.) or a self-antigen, on the surface of the at least one cancer cell. In some embodiments, the antigen on the surface of the at least one cancer cell is a tumor antigen.

Method of Treating a Subject with Cancer

In one embodiment, the present invention relates to methods and compositions for treating or preventing cancer in a subject.

In one embodiment, the methods for treating or preventing cancer in a subject comprising administering one or more T cells of the present invention having enhanced features to the subject. In one embodiment the one or more T cells having enhanced features is further modified to express a chimeric antigen receptor (CAR), wherein the CAR binds to a cell surface antigen on at least one tumor cell in the body subject.

In one embodiment, the methods for treating or preventing cancer in a subject comprise obtaining one or more T cells from the subject, generating one or more T cells having enhanced features from the one or more T cells from the subject, and administering the one or more T cells having enhanced features to the subject. In one embodiment, the one or more T cells comprise one or more tumor infiltrating cells (TILs). In one embodiment, the method further comprises modifying the one or more T cells having enhanced features to express a CAR, wherein the CAR binds to a cell surface antigen on at least one tumor cell.

### Compositions

T Cells with Enhanced Features

In one embodiment, the invention comprises compositions comprising one or more T cells with enhanced features. In one embodiment, the invention comprises compositions comprising an enriched population of T cells with enhanced features, wherein the enriched population of T cells with enhanced features is identified or generated by the methods of the present invention.

### Sources of T Cells

In one embodiment, the invention comprises methods and compositions relating to one or more T cells having enhanced features.

In one embodiment, the one or more T cells comprise CD8+ T cells. In one embodiment, the T cells comprise Pmel CD8+ T cells.

In one embodiment the one or more T cells comprises CD4+ T cells, CD4+ T cells, tumor infiltrating lymphocytes (TILs), central memory T cells (T_{CM}), and any combination thereof. In one embodiment, the one or more T cells is a TIL isolated from the tumor of subject with cancer.

Prior to expansion, a source of T cells is obtained from a subject. Examples of subjects include humans, dogs, cats, mice, rats, and transgenic species thereof. Preferably, the subject is a human. T cells can be obtained from a number of sources, including peripheral blood mononuclear cells, bone marrow, lymph node tissue, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many or all divalent cations. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood by lysing the red blood cells and depleting the monocytes, for example, by centrifugation through a PERCOLL™ gradient. Alternatively, T cells can be isolated from umbilical cord. In any event, a specific subpopulation of T cells can be further isolated by positive or negative selection techniques.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations.

### Antigen Administration

In one embodiment, the one or more T cells with enhanced features of the present invention can be generated to be reactive to any desirable tumor antigen of interest. In one embodiment, the one or more T cells are administered one or more tumor antigens. In one embodiment, the administration comprises activation of the one or more T cells.

In one embodiment, administration of the one or more tumor antigens occurs prior to identifying or generating of one or more T cells with enhanced features as described elsewhere herein. In one embodiment, administration of the one or more tumor antigens occurs after identifying or generating the one or more T cells with enhanced features as described elsewhere herein. In one embodiment, administration of the one or more tumor antigens occurs during the procedure for identifying or generating one or more T cells with enhanced features as described elsewhere herein.

In the context of the present invention, “tumor antigen” or “hyperproliferative disorder antigen” or “antigen associated with a hyperproliferative disorder,” refers to antigens that are common to specific hyperproliferative disorders such as cancer. The antigens discussed herein are merely included by way of example. The list is not intended to be exclusive and further examples will be readily apparent to those of skill in the art.

Tumor antigens are proteins that are produced by tumor cells that elicit an immune response. The selection of the antigen binding domain of the invention will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In one embodiment, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

Depending on the desired antigen to be targeted, the cells of the invention can be modified to target the appropriate antigen.

In one embodiment, the one or more tumor antigens comprise gp100.

In one embodiment, the one or more tumor antigens are at a concentration of 0.5-1 μg/ml. In one embodiment, the one or more tumor antigens at a concentration of 1-1.5 μg/ml. In one embodiment, the one or more tumor antigens are at a concentration of 1.5-2 μg/ml. In one embodiment, the one or more tumor antigens are at a concentration of 2.5-3 μg/ml. In one embodiment, the one or more tumor antigens are at a concentration of 1 μg/ml.

In one embodiment, the one or more T cells are administered the one or more tumor antigens for 1-2 days. In one embodiment, the one or more T cells are administered the one or more tumor antigens for 2-3 days. In one embodiment, the one or more T cells are administered the one or more tumor antigens for 3-4 days. In one embodiment, the one or more T cells are administered the one or more tumor antigens dies for 4-5 days. In one embodiment, the one or more T cells are administered the one or more tumor antigens for 5-6 days. In one embodiment, the one or more T cells are administered the one or more tumor antigens for 6-7 days. In one embodiment, the one or more T cells are administered the one or more tumor antigens for 3 days.

### Cytokine Administration

In one embodiment, the one or more T cells are administered one or more cytokines. In one embodiment, administration of one or more cytokines comprises activation of the one or more T cells.

In one embodiment, administration of one or more cytokines occurs prior to identifying or generating of one or more T cells with enhanced features as described elsewhere herein. In one embodiment, administration of one or more cytokines occurs after identifying or generating the one or more T cells with enhanced features as described elsewhere herein. In one embodiment, administration of one or more cytokines occurs during the procedure for identifying or generating one or more T cells with enhanced features as described elsewhere herein.

In one embodiment, the one or more cytokines comprises IL-2, IL-15, or any combination thereof.

In one embodiment, the one or more cytokines are at a concentration of 1-2 ng/ml. In one embodiment, the one or more cytokines at a concentration of 2-3 ng/ml. In one embodiment, the one or more cytokines are at a concentration of 3-4 ng/ml. In one embodiment, the one or more cytokines are at a concentration of 4-5 ng/ml. In one embodiment, the one or more cytokines are at a concentration of 5-6 ng/ml. In one embodiment, the one or more cytokines are at a concentration of 6-7 ng/ml. In one embodiment, the one or more cytokines are at a concentration of 7-8 ng/ml. In one embodiment, the one or more cytokines are at a concentration of 8-9 ng/ml. In one embodiment, the one or more cytokines are at a concentration of 9-10 ng/ml. In one embodiment, the one or more cytokines are at a concentration of 10-11 ng/ml. In one embodiment, the one or more cytokines are at a concentration of 11-12 ng/ml. In one embodiment, the one or more cytokines are at a concentration of 10 ng/ml.

In one embodiment, the one or more cytokines are at a concentration of 20-40 IU/ml. In one embodiment, the one or more cytokines at a concentration of 40-60 IU/ml. In one embodiment, the one or more cytokines are at a concentration of 60-80 IU/ml. In one embodiment, the one or more cytokines are at a concentration of 80-100 IU/ml. In one embodiment, the one or more cytokines are at a concentration of 100-120 IU/ml. In one embodiment, the one or more cytokines are at a concentration of 120-140 IU/ml. In one embodiment, the one or more cytokines are at a concentration of 140-160 IU/ml. In one embodiment, the one or more cytokines are at a concentration of 160-180 IU/ml. In one embodiment, the one or more cytokines are at a concentration of 180-200 IU/ml. In one embodiment, the one or more cytokines are at a concentration of 200-220 IU/ml. In one embodiment, the one or more cytokines are at a concentration of 220-240 IU/ml. In one embodiment, the one or more cytokines are at a concentration of 100 IU/ml. In one embodiment, the one or more cytokines are at a concentration of 200 IU/ml.

In one embodiment, the one or more T cells are administered the one or more cytokines for 1-2 days. In one embodiment, the one or more T cells are administered the one or more cytokines for 2-3 days. In one embodiment, the one or more T cells are administered the one or more cytokines for 3-4 days. In one embodiment, the one or more T cells are administered the one or more cytokines for 4-5 days. In one embodiment, the one or more T cells are administered the one or more cytokines for 5-6 days. In one embodiment, the one or more T cells are administered the one or more cytokines for 6-7 days. In one embodiment, the one or more T cells are administered the one or more cytokines for 3 days.

### Antibody Administration

In one embodiment, the one or more T cells are administered one or more antibodies. In one embodiment, the administration comprises activation of the one or more T cells.

In one embodiment, administration of one or more antibodies occurs prior to identifying or generating of one or more T cells with enhanced features as described elsewhere herein. In one embodiment, administration of one or more antibodies occurs after identifying or generating the one or more T cells with enhanced features as described elsewhere herein. In one embodiment, administration of one or more antibodies occurs during the procedure for identifying or generating one or more T cells with enhanced features as described elsewhere herein.

In one embodiment, the one or more antibodies comprises anti-CD3, anti-CD28, or any combination thereof.

In one embodiment, the one or more antibodies are at a concentration of 0.5-1 μg/ml. In one embodiment, the one or more antibodies at a concentration of 1-1.5 μg/ml. In one embodiment, the one or more antibodies are at a concentration of 1.5-2 μg/ml. In one embodiment, the one or more antibodies are at a concentration of 2.5-3 μg/ml. In one embodiment, the one or more antibodies are at a concentration of 1 μg/ml.

In one embodiment, the one or more T cells are administered the one or more antibodies for 1-2 days. In one embodiment, the one or more T cells are administered the one or more antibodies for 2-3 days. In one embodiment, the one or more T cells are administered the one or more antibodies for 3-4 days. In one embodiment, the one or more T cells are administered the one or more antibodies for 4-5 days. In one embodiment, the one or more T cells are administered the one or more antibodies for 5-6 days. In one embodiment, the one or more T cells are administered the one or more antibodies for 6-7 days. In one embodiment, the one or more T cells are administered the one or more antibodies for 3 days.

Expression of Nucleic Acids and/or Proteins in Cells

A variety of methods can be used to express or overexpress nucleic acids and/or proteins in the one or more T cells of the present invention.

In some embodiments, nucleic acids can be cloned into a number of types of vectors which are then introduced into cells. However, the present invention should not be construed to be limited to any particular vector. Instead, the present invention should be construed to encompass a wide variety of vectors which are readily available and/or known in the art. For example, the nucleic acid of the invention can be cloned into a vector including, but not limited to a plasmid, a phagemid, a phage derivative, an animal virus, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available. Viral vector technology is well known in the art and is described, for example, in Sambrook et al. (2012), and in Ausubel et al. (1999), and in other virology and molecular biology manuals. Viruses, which are useful as vectors include, but are not limited to, retroviruses, adenoviruses, adeno-associated viruses, herpes viruses, and lentiviruses. In some embodiments, a murine stem cell virus (MSCV) vector is used to express a desired nucleic acid. MSCV vectors have been demonstrated to efficiently express desired nucleic acids in cells. However, the invention should not be limited to only using a MSCV vector, rather any retroviral expression method is included in the invention. Other examples of viral vectors are those based upon Moloney Murine Leukemia Virus (MoMuL V) and human immunodeficiency virus (HIV). In some embodiments, a suitable vector contains an origin of replication functional in at least one organism, a promoter sequence, convenient restriction endonuclease sites, and one or more selectable markers.

Additional regulatory elements, e.g., enhancers, can be used modulate the frequency of transcriptional initiation. A promoter may be one naturally associated with a gene or nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” e.g., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein.

Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high-level expression of the introduced DNA segment, such as is advantageous in the large-scale production of recombinant proteins and fragments thereof.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV 40) early promoter, mouse mammary tumor virus (MMTV), HIV long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter or cell-type specific promoter, which is a promoter that is active only in a desired tissue or cell. Tissue-specific promoters are well known in the art and include, but are not limited to, the HER-2 promoter and the PSA associated promoter sequences.

In order to assess the expression of the nucleic acids, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate nucleic acid and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Methods of introducing and expressing nucleic acids into a cell are known in the art. In the context of an expression vector, the vector can be readily introduced into a host cell, e.g., mammalian, bacterial, yeast or insect cell by any method in the art. For example, the expression vector can be transferred into a host cell by physical, chemical or biological means.

Physical methods for introducing a polynucleotide into a host cell include calcium phosphate precipitation, lipofection, particle bombardment, microinjection, electroporation, laserporation and the like. Methods for producing cells comprising vectors and/or exogenous nucleic acids are well-known in the art. See, for example, Sambrook et al. (2012) and Ausubel et al. (1999).

Biological methods for introducing a nucleic acid of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like.

Chemical means for introducing a nucleic acid into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (e.g., an artificial membrane vesicle). The preparation and use of such systems is well known in the art.

In some embodiments, proteins can be delivered directly into cells by a variety of methods well known in the field to delivery proteins intracellularly. In some embodiments, such methods include intracellular delivery of proteins by electroporation, intracellular delivery of proteins via lipid nanoparticle, intracellular delivery of proteins by membrane perforation methodologies, extracellular vesicles and cell-penetrating peptides based systems.

Regardless of the method used to introduce exogenous nucleic acids and/or proteins into a host cell or otherwise administer to a cell the nucleic acid and/or protein of the present invention, in order to confirm the presence of the recombinant DNA sequence and/or protein in the host cell, a variety of assays may be performed. Such assays include, for example, “molecular biological” assays well known to those of skill in the art, such as Southern and Northern blotting, Western blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Therapeutic Application

In one embodiment, the present invention includes a type of cellular therapy using the one or more T cells with enhanced features described herein. In one embodiment, the method comprises administering a T cell described herein to a subject having cancer. In one embodiment, the method comprises administering a T cell described herein to a subject having a viral infection.

In one embodiment, the one or more T cells with enhanced features described herein can be infused to a recipient in need thereof. In one embodiment, the infused cell is able to kill tumor cells in the recipient. Unlike antibody therapies, the one or more T cells with enhanced features of the invention are able to result in long-term persistence that can lead to sustained tumor control.

In another embodiment, the present invention includes a type of cellular therapy wherein the one or more T cells with enhanced features are additionally administered an agent that further alters the characteristics of the one or more T cells with enhanced features. In one embodiment, one or more T cells with enhanced features can then be infused to a recipient in need thereof. In one embodiment, the infused cell is able to kill tumor cells in the recipient. In one embodiment, the infused cell is able to reduce a viral load in the recipient.

In one embodiment, the one or more T cells with enhanced features of the invention exhibiting high anti-oxidant capacity are able to promote differentiation and maintenance of memory T cells (Tcm) in oxidative tumor microenvironments. In one embodiment, the one or more T cells with enhanced features of the invention are able to generate anti-tumor Tcm phenotype cells in vivo.

In another embodiment, the one or more T cells with enhanced features of the invention evolve into specific memory T cells that can be reactivated to inhibit any additional tumor formation or growth. For example, the cells of the invention exhibit persistence and increased anti-tumor activity. Without wishing to be bound by any particular theory, the one or more T cells with enhanced features of the invention may differentiate in vivo into a central memory-like state upon encounter and subsequent elimination of target cells expressing the surrogate antigen.

Without wishing to be bound by any particular theory, the anti-tumor immunity response elicited by the one or more T cells with enhanced features of the invention may be an active or a passive immune response. In addition, the immune response may be part of an adoptive immunotherapy approach in which T cells induce an immune response specific to a desired antigen.

Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the CARs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, meningioma, neuroblastoma, retinoblastoma and brain metastases).

The one or more T cells with enhanced features of the invention may also serve as a type of vaccine for ex vivo immunization and/or in vivo therapy in a mammal. Preferably, the mammal is a human.

Ex vivo procedures are well known in the art and are discussed more fully below. Briefly, cells are isolated from a mammal (preferably a human) and genetically (i.e., transduced or transfected in vitro) or biochemically (i.e., treated with an agent, such as SP600125) modified. The modified cell can be administered to a mammalian recipient to provide a therapeutic benefit. The mammalian recipient may be a human and the modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient.

### Pharmaceutical

In some embodiments, the present invention relates to pharmaceutical compositions comprising the one or more T cells with enhanced features of the present invention.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “a tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 104 to 109 cells/kg body weight, preferably 105 to 106 cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol may serve to select out certain populations of T cells.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the T cell compositions of the present invention are administered to a patient by intradermal or subcutaneous injection. In another embodiment, the T cell compositions of the present invention are preferably administered by i.v. injection. The compositions of T cells may be injected directly into a tumor, lymph node, or site of infection.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Modulating the ER and Golgi Stress Response in T Cells with Hydrogen Sulfide Signaling to Enhance the Anti-Tumor Immune Response

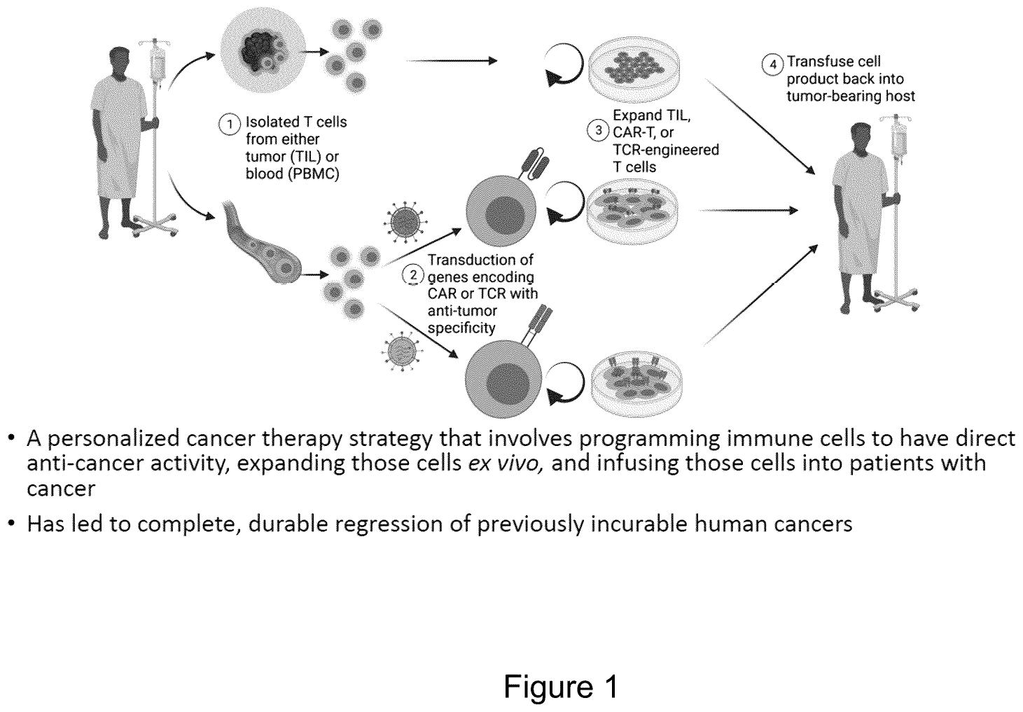

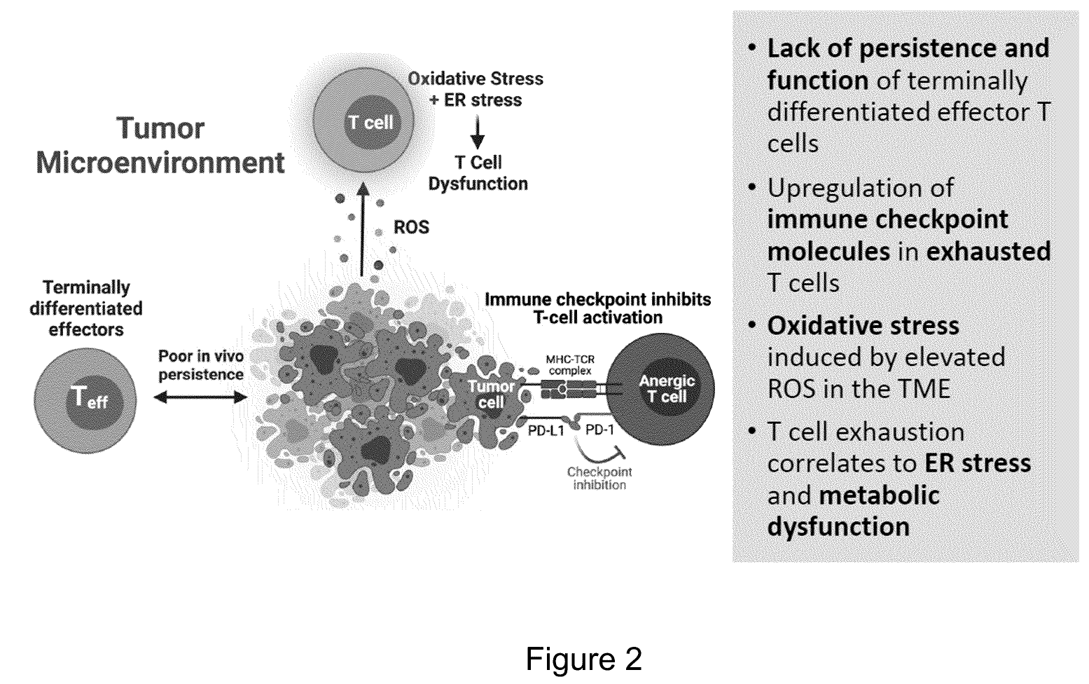

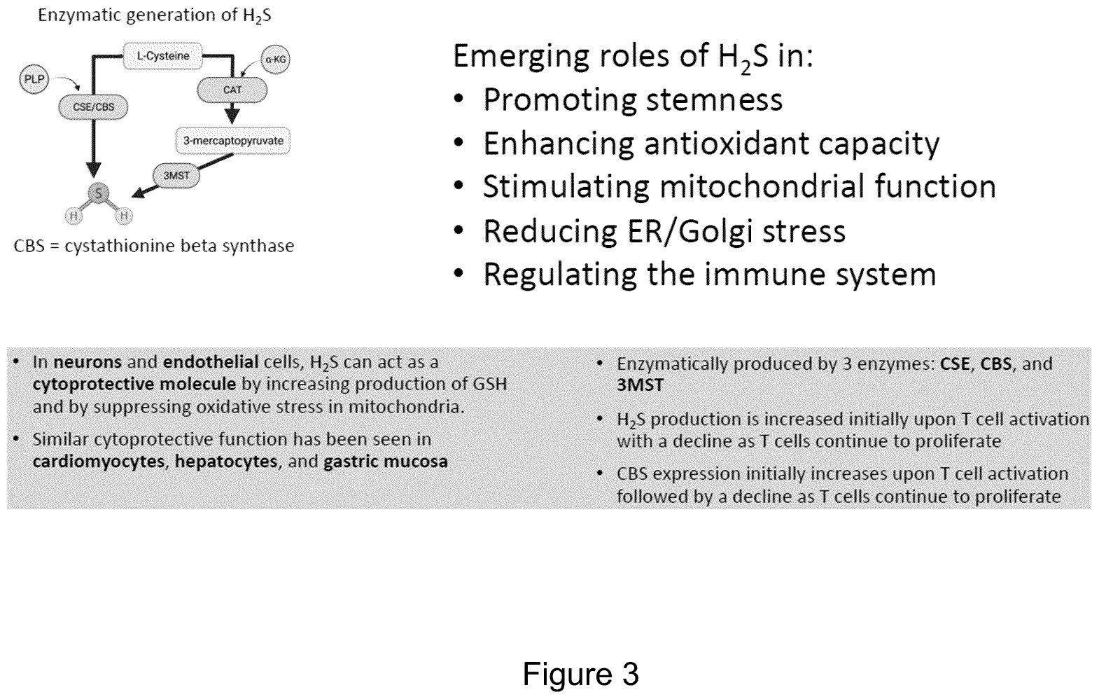

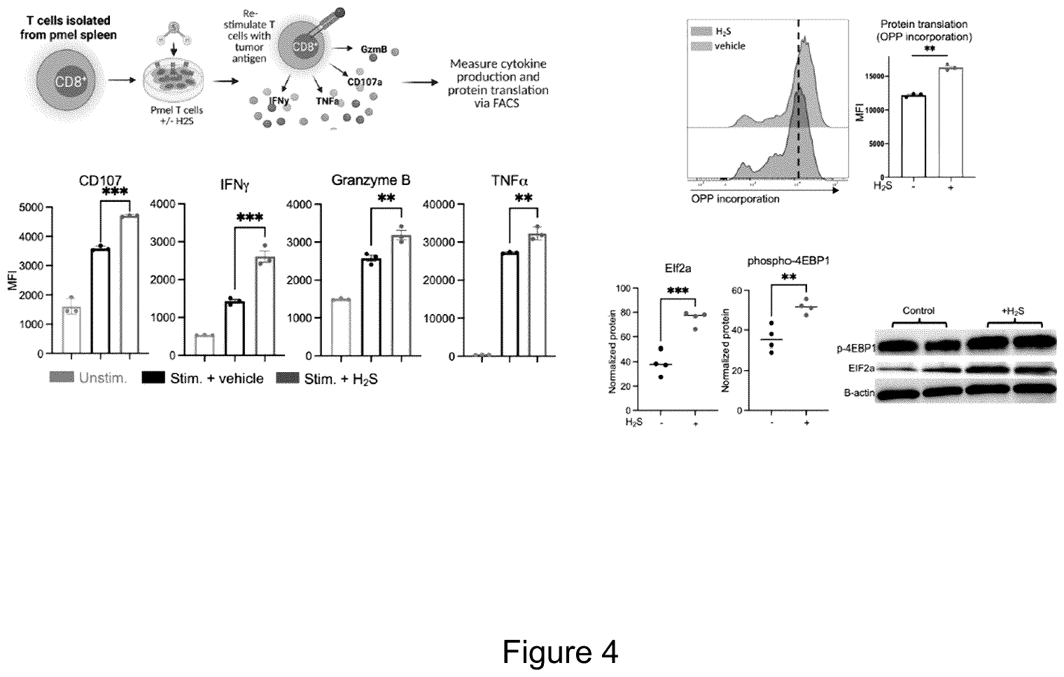

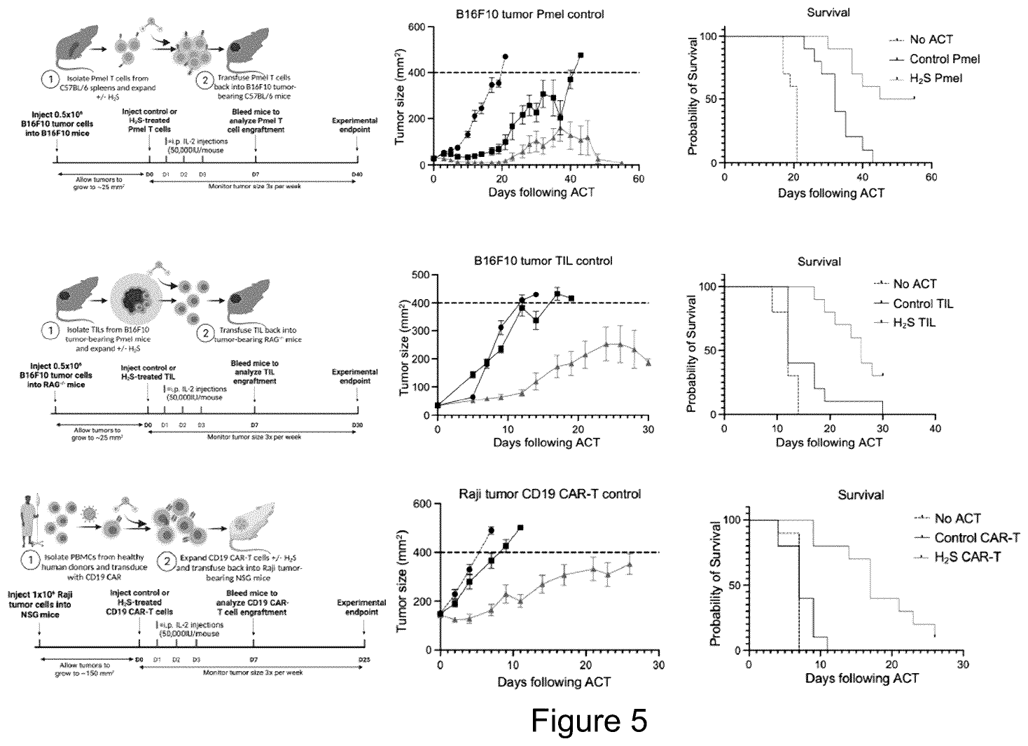

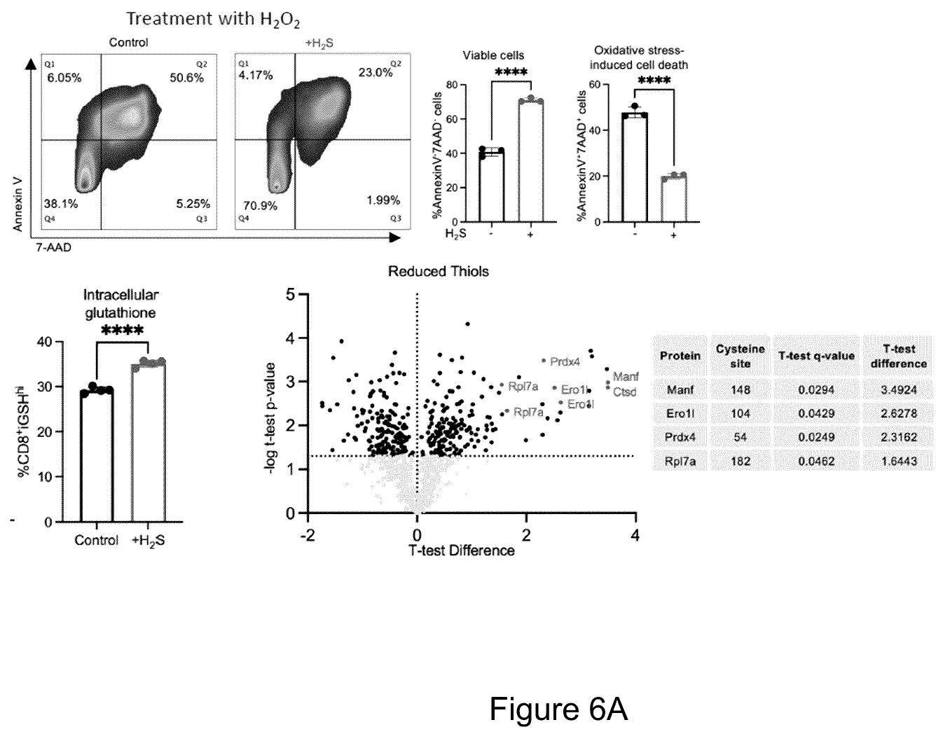

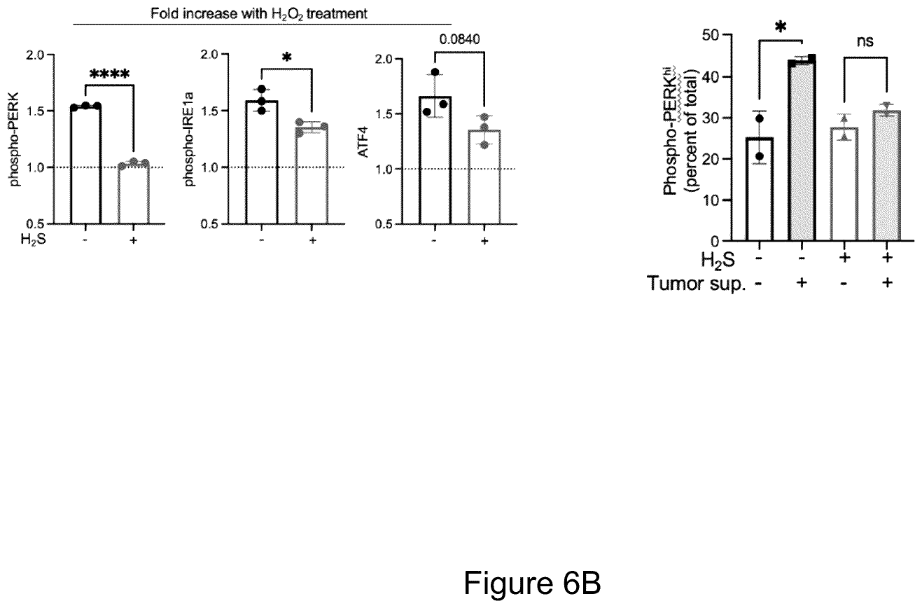

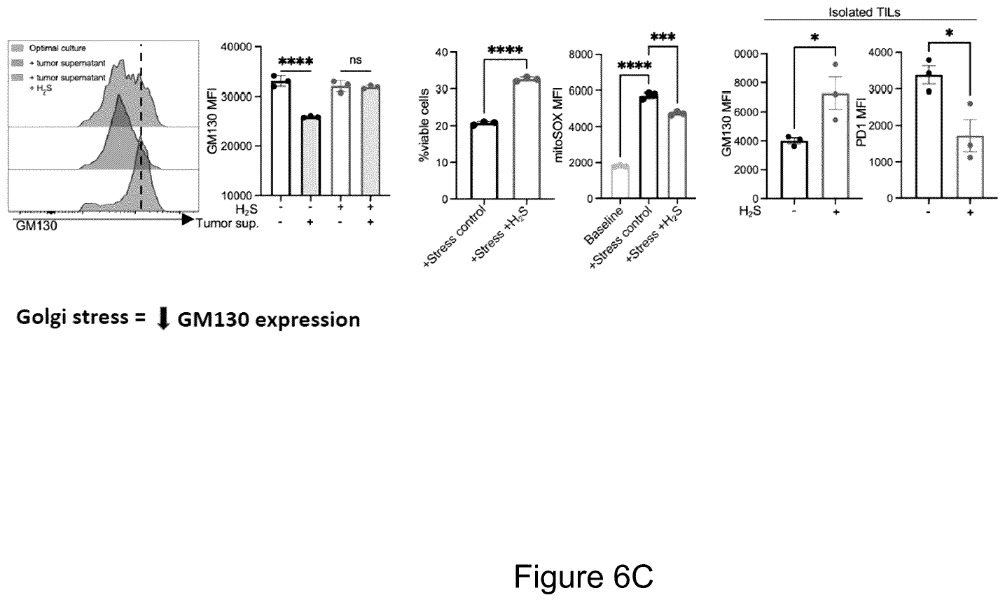

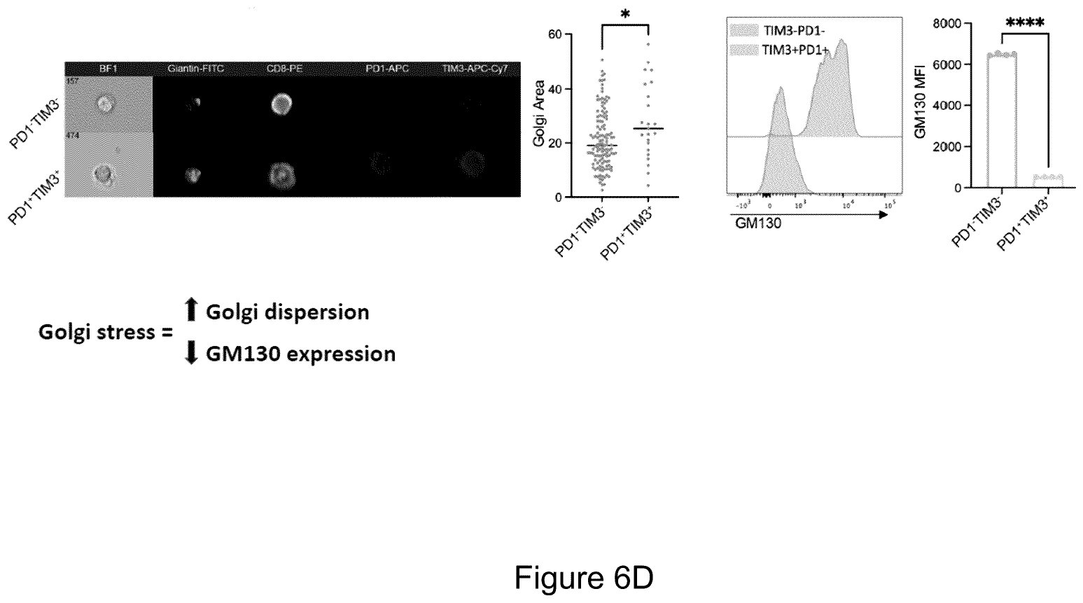

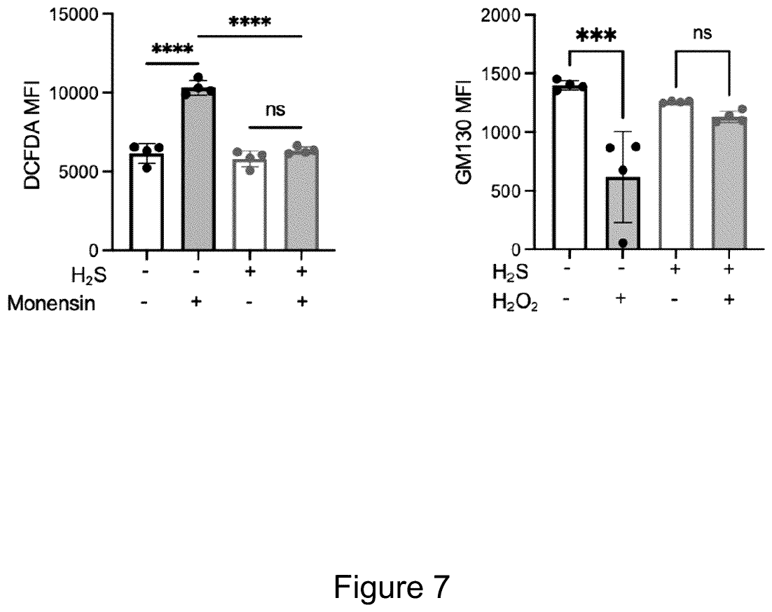

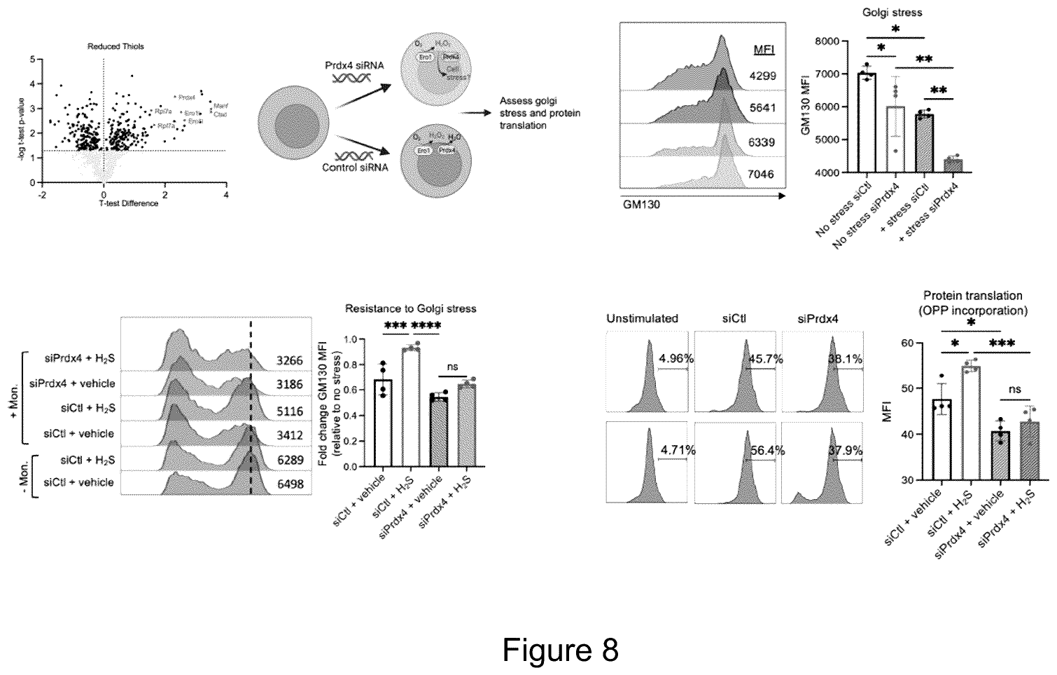

Adoptive cell therapy (ACT) continues to emerge as a novel therapeutic strategy for treating cancer (FIG. 1). New approaches to improve ACT protocols are needed to enhance in vivo persistence of adoptively transferred tumor antigen-specific T cells and overcome tumor-induced immunosuppression and cellular stress (FIG. 2). Hydrogen sulfide (H₂S) is a cryoprotective signaling molecule (FIG. 3). Here, it is shown that H₂S supports T cell effector function and protein translation (FIG. 4). It is further shown that H₂S enhanced antitumor efficacy (FIG. 5). It is additional shown that H₂S reduced oxidative stress and stress within the Golgi-ER axis in antitumor cells (FIG. 6).

### Example 2: Cellular Reprogramming by Targeting the Golgi Apparatus

Based on the findings that Golgi stress is induced in T cells in the setting of the TME with a correlated decrease in GM130 levels, the phenotype and function of T cells displaying high versus low Golgi mass was characterized.

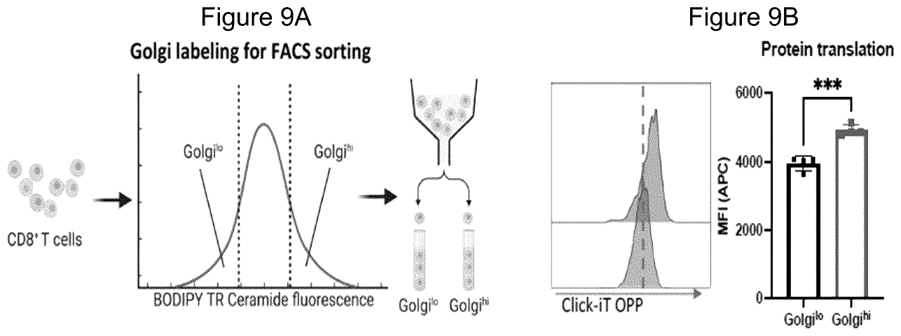

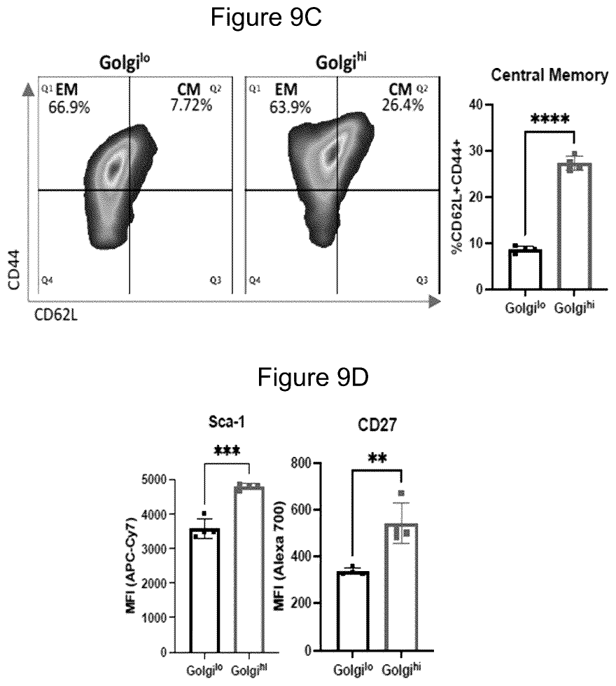

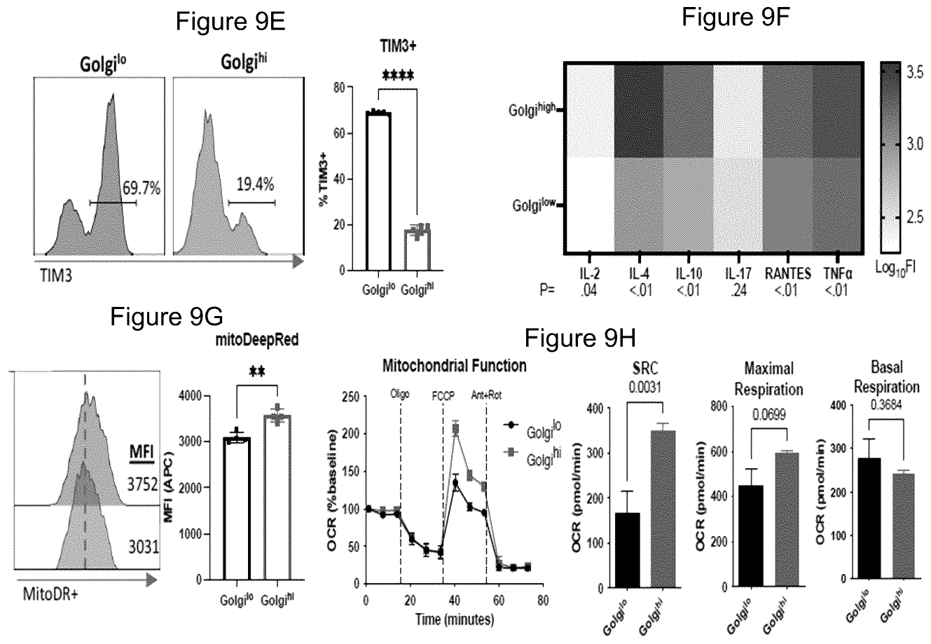

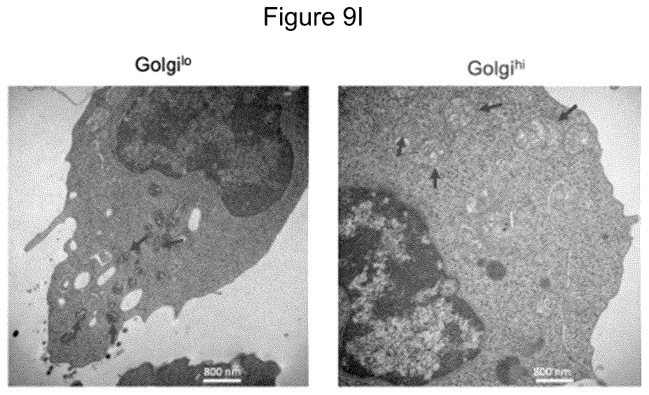

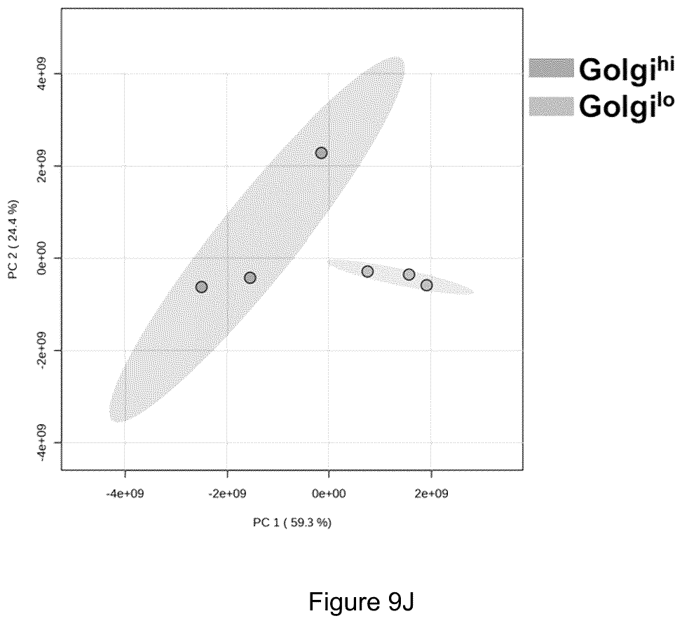

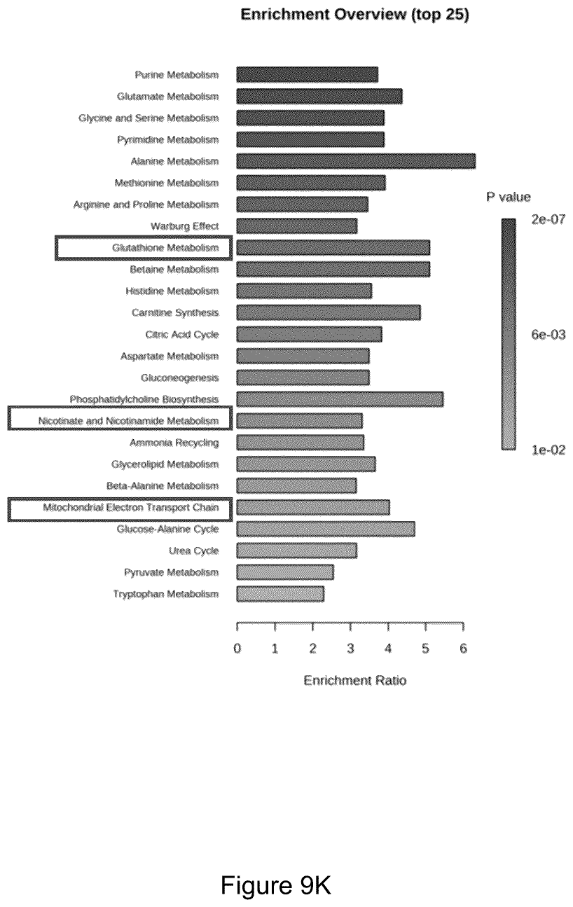

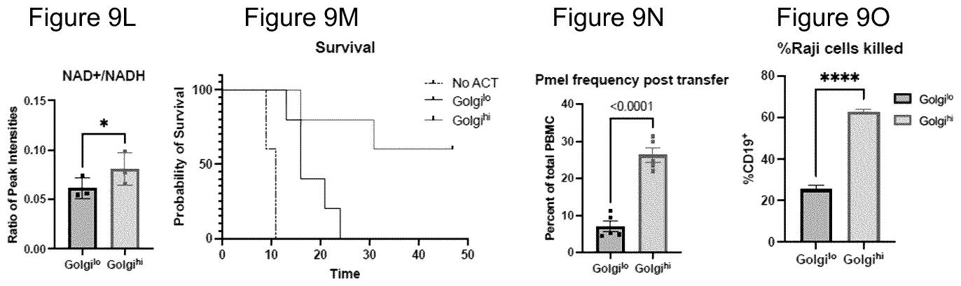

The Golgi of activated Pmel CD8+ T cells were stained using a fluorescent dye and sorted into cells possessing high Golgi mass (Golgi^{hi}) and cells possessing low Golgi mass (Golgi^{lo}) (FIG. 9A). Protein translation was significantly higher in the T cells possessing high Golgi mass upon re-stimulation with gp100 tumor antigen compared to the T cells possessing low Golgi mass (FIG. 9B). Interestingly, Golgi mass was also directly correlated with the Tcm phenotype, with Golgi^{hi} cells having a higher proportion of CD62L+CD44+ cells (FIG. 9C) and higher expression of Sca-1 and CD27 (FIG. 9D). In the TME exhaustion assay, Golgi^{lo} cells had a significantly higher proportion of cells expressing TIM3 compared to Golgi^{hi} cells, indicating a resistance to T cell exhaustion in the Golgi^{hi} population (FIG. 9E).

Functionally, Golgi^{hi} cells produced more proinflammatory cytokines upon restimulation with tumor antigen, including IL-2, IL-4, RANTES, and TNFa (FIG. 9F).

Golgi^{hi} cells also were also characterized by a significant increase in mitochondrial mass compared to Golgi^{lo} (FIG. 9G). Similar to H₂S-treated T cells, Golgi^{hi} T cells also displayed a significant increase in spare respiratory capacity along with a slight increase in maximal respiration, indicating a correlation between Golgi^{hi} cells and enhanced mitochondrial fitness (FIG. 9H). Transmission electron microscopy (TEM) analysis of Golgi^{hi} versus Golgi^{lo} cells revealed large, healthy-appearing mitochondria in Golgi^{hi} cells compared to small, dense mitochondria in the Golgi^{lo} cells (FIG. 9I).

Metabolomics analysis revealed significant differences in the metabolite profiles of Golgi^{hi} versus Golgi^{lo} cell (FIG. 9J). Of note, several key metabolic pathways were found to be upregulated in the Golgi^{hi} cells compared to Golgi^{lo} cells, including glutathione metabolism, nicotinate/nicotinamide metabolism, and mitochondrial electron transport train (FIG. 9K). Similar to H₂S-treated T cells, Golgi^{hi} cells had a significantly higher NAD+/NADH ratio (FIG. 9L).

The antitumor capacity of Golgi^{hi} versus Golgi^{lo} cells was determined, hypothesizing that Golgi^{hi} cells would exert superior tumor control when adoptively transferred into tumor-bearing hosts. Indeed, it was found that Golgi^{hi} Pmel T cells displayed significantly better overall tumor control (FIG. 9M) and better engraftment in B16F10-bearing C57BL/6 mice (FIG. 9N). Additionally, when human CD19 CAR-T cells were sorted into Golgi^{hi} and Golgi^{lo} populations and co-cultured with Raji cells, the Golgi^{hi} CAR-T cells displayed superior killing ability, confirming that sorting T cells based on the status of the Golgi apparatus identifies T cells with superior ability to control tumor cells (FIG. 9O).

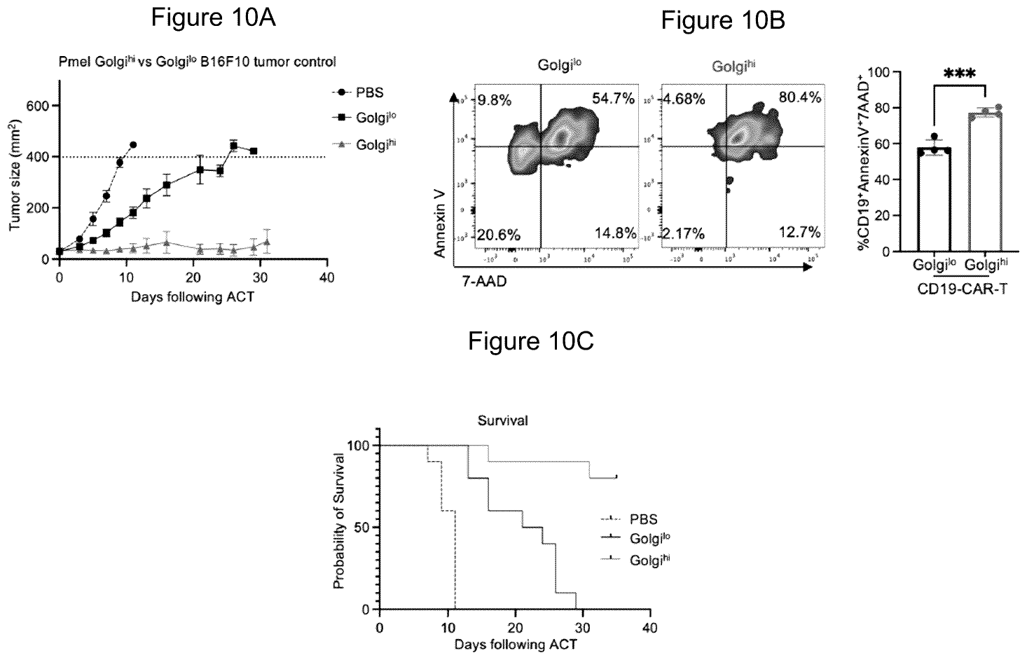

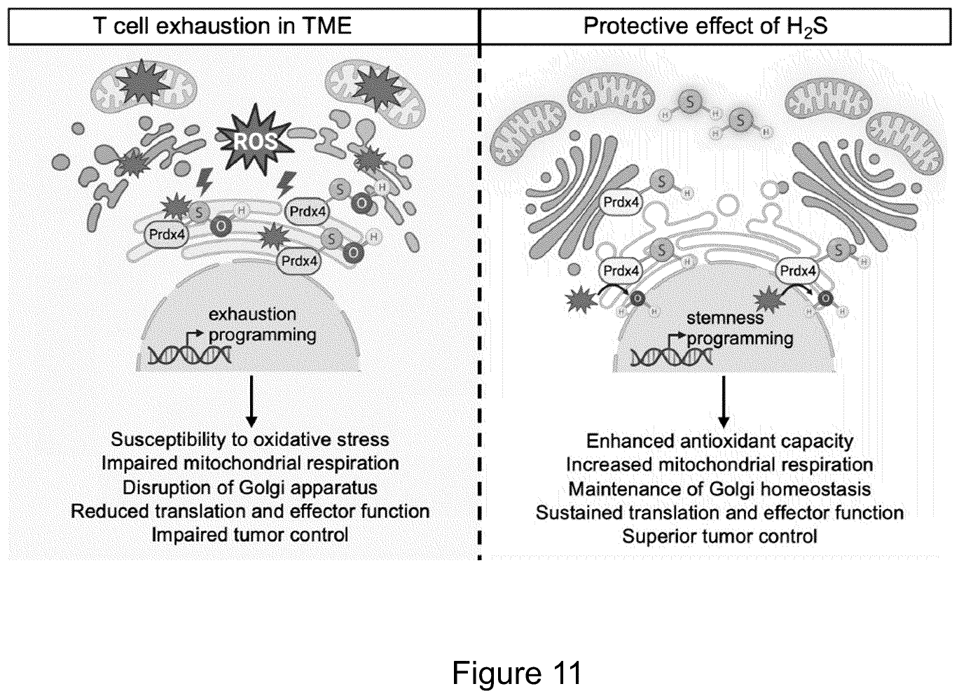

The antitumor capacity of Golgi^{hi} versus Golgi^{lo} cells was determined in vivo and it was found that Golgi^{hi} cells would exert superior tumor control when adoptively transferred into tumor-bearing hosts (FIG. 10A). When Human CD19 CAR-T cells were sorted into Golgi^{hi} and Golgi^{lo} populations and compared for AnnexinV and 7-ADD expression, it was found that a greater percentage of Golgi^{hi} cells express AnnexinV and 7-ADD (FIG. 10B). Greater probability of survival was observed following ACT of Golgi^{hi} compared to Golgi^{lo} or PBS only control (FIG. 10C).

### Example 3: H₂S-Prdx4 Axis Mitigates Golgi Stress to Bolster Tumor-Reactive T Cell Immunotherapeutic Response

The role of tumor microenvironment (TME) associated inadequate protein modification and trafficking due to insufficiency in Golgi function, leading to Golgi stress, in the regulation of T cell function is largely unknown. Here it is shown that disruption of Golgi architecture under TME stress, identified by the decreased expression of GM130, was reverted upon treatment with hydrogen sulfide (H₂S) donor GYY4137 or over-expressing cystathionine β-synthase (CBS), an enzyme involved in the biosynthesis of endogenous H₂S, which also promoted stemness, antioxidant capacity and increased protein translation, mediated in part by ER-Golgi shuttling of Peroxiredoxin-4. In in vivo models of melanoma and lymphoma, anti-tumor T cells conditioned ex vivo with exogenous H₂S or overexpressing Cbs demonstrated superior tumor control upon adoptive transfer. Further, T cells with high Golgi content exhibited unique metabolic and glycation signatures with enhanced anti-tumor capacity. These data suggest that strategies to mitigate Golgi network stress or using Golgi^{hi} tumor-reactive T cells can improve tumor control upon adoptive transfer.

Adoptive transfer of tumor reactive T cells has shown promising results in metastatic melanoma and advanced B cell malignancies (Rosenberg S. A. et al., 2015, Science, 348(6230):62-68; Chakraborty P. et al., 2019, J Biol Chem., 294(23):9198-9212). However, a quantitative or qualitative decrease of the transferred anti-tumor T cells in the tumor bearing host typically results in tumor recurrence, leaving substantial room for improvement (Topalian S. L. et al., 2015, Cell, 161(2):185-6; Rosenberg S. A. et al., 2012, Sci Transl Med., 4(127):127ps8-127ps8; Phan G. Q. et al., 2013, Cancer Control., 20(4):289-97; Gajewski T. F. et al., 2013, Curr Opin Immunol., 25(2):268-76). Strategies to improve anti-tumor T cell function by altering mitochondrial bioenergetics (Sukumar M. et a., 2013, J Clin Invest., 123(10):4479-88; Sukumar M., et al., 2016, Cell Metab., 23(1):63-76) or its metabolites (Chatterjee S. et al., 2018, Cell Metab., 27(1):85-100), mitigating ER-stress (Katoh Y. et al., 2022, J Immunother Cancer., 10(7)), or inducing protective autophagy (Chakraborty P. et al., 2022, Cancer Research, 82(10):1969-1990) are being widely tested. Multiple studies have identified oxidative and ER stress within the TME as major contributors of immune cell dysfunction and immune evasion (Aboelella N. S. et al., 2021, Cancers (Basel), 2021; 13(5):986; Chen X. et a., 2016, Oxid Med Cell Longev., 1580967; Kotsafti A., et al., 2020, Cancers (Basel)., 12(7):1748; Malmberg K. J. et al., 2001, 167(5):2595-601; Ma Y., et al., 2024, 24(4):264-281). Similarly, the mammalian Golgi apparatus not only serves important roles in the transport, processing, and targeting of proteins, but when under stress mounts a stress response where its unique structure can be fine-tuned to adapt different Golgi functions to specific cellular needs (Zhang Y., et al., 2019, 32(9):583-601; Bui S., et al., 2021, Front Cell Dev Biol., 9:806482). While the synchronized activity of these cellular organelles is being increasingly recognized for maintaining quality control and ensuring cell survival and function (Rossini M., et a., 2021, Febs J., 288(3):740-755), specifics of Golgi dynamics in the tumor microenvironment (TME) and the role of the Golgi stress response in shaping T cell function have thus far been understudied.

Similar to a recent study where carbon monoxide mediated transient activation of the ER stress PERK pathway led to increased mitochondrial biogenesis and reprogramming of anti-tumor T cells to effectively treat established tumors upon adoptive T cell transfer (Chakraborty P., et al., 2022, 82(10):1969-1990), transient activation of Golgi stress mediated by monensin was shown to stimulate the reverse trans-sulfuration pathway via cystathionine γ-lyase (CSE) (the biosynthetic enzyme for cysteine and an important regulator of redox homeostasis) to mitigate the toxicity associated with cysteine deprivation in Huntington's disease (HD) (Sbodio J. I., et al., 2018, PNAS, 115(4):780-785). Given that CSE, cystathionine β-synthase (CBS), and 3-mercaptopyruvate sulfurtransferase (3-MST) mediated secretion of hydrogen sulfide (H₂S), an endogenous signaling gaseous transmitter that also mitigates Golgi stress (Zhang Y., et al., 2019, Antioxidants & Redox Signaling, 32(9):583-601), has been shown to regulate the immune response immune in mammals (Dilek N., et al., 2020, Pharmacological Research, 161:105119), it was hypothesized that H₂S may establish a reduced stress state in anti-tumor T cells and that increasing H₂S could potentiate the anti-tumor T cell response by mitigating ER and Golgi stress.

Here, it is shown that T cell intrinsic H₂S signaling supports overall protein translation and improves T cell effector function by reducing ER and Golgi stress. The levels of H₂S or cystathionine-β-synthase (Cbs) also inversely correlated with exhaustion, and replenishing H₂S exogenously during T cell activation or expansion led to an increase in the central memory (Tcm) phenotype by engaging the NAD+-Sirt1-Foxo1 axis. Proteomics analysis highlighted that increased abundance of free thiols in Peroxiredoxin-4 (Prdx4) was also in part responsible for the H₂S mediated Tcm phenotype. Importantly, ER-localized Prdx4 was found to translocate to the Golgi under conditions of oxidative stress. Further, delineating T cells based on Golgi content highlighted that the T cell subsets with high Golgi content (Golgi^{hi} subset) exhibit long-term tumor control upon adoptive transfer. Further, human CD19 chimeric antigen receptor (CAR) T cells overexpressing Cbs or sorted for Golgi^{hi} significantly increased the survival of human lymphoma xenografted mice compared to mice treated with Golgi^{lo} CAR-T cells. Collectively, these results not only provide insight into the important role of endogenous H₂S in regulating the T cell immune response but also highlight the Golgi network as a novel therapeutic target for enhancing the efficacy of immunotherapy for cancer.

### Results

### H₂S Promotes Generation of Central Memory (Tcm) Anti-Tumor T Cells.

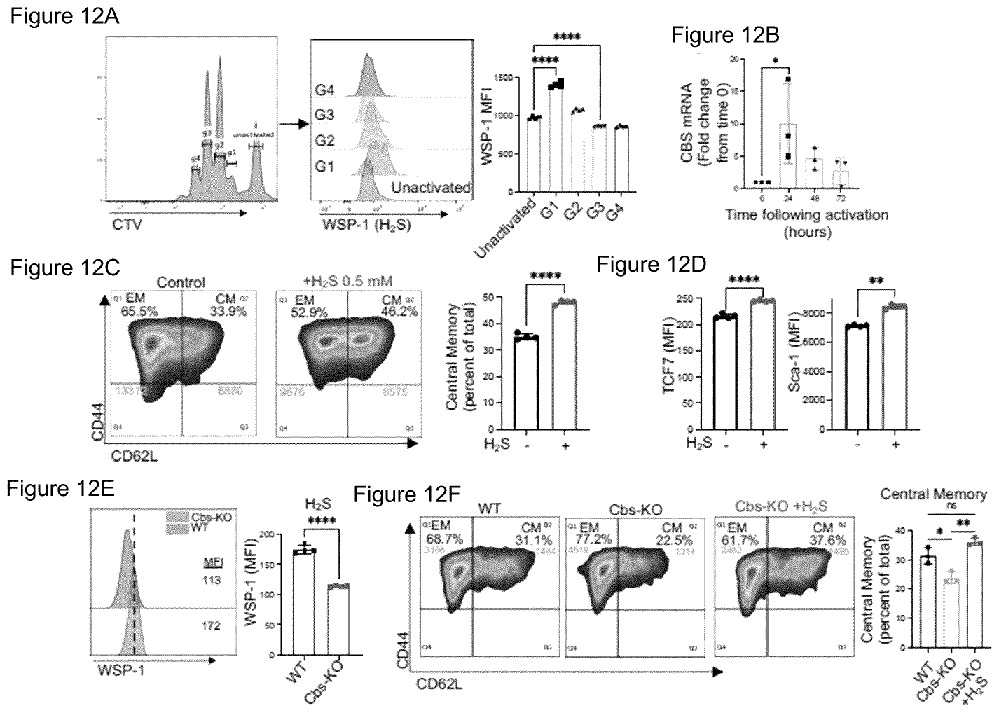

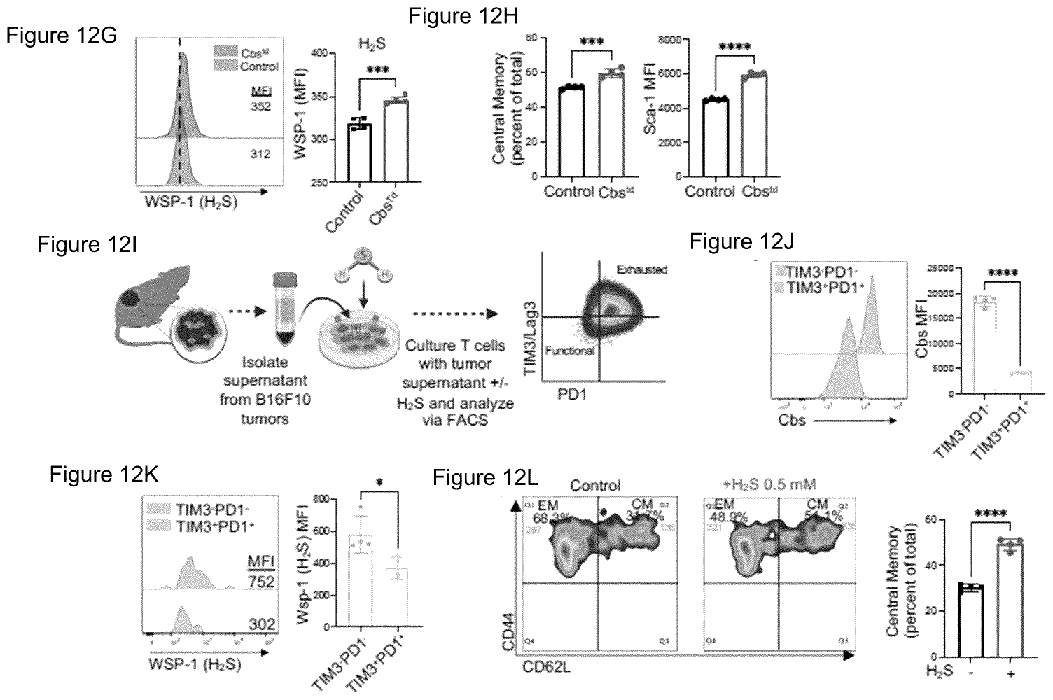

Given the limited knowledge on the role of H₂S in primary T cells, the kinetics of endogenous H₂S production in anti-tumor T cells during T cell receptor (TCR) mediated activation was determined. Melanoma epitope gp100 reactive CD8+ T cells (from Pmel-transgenic mouse spleen) were stained with Cell Trace Violet (CTV) proliferation dye and activated in vitro with gp100 cognate antigen. After 3 days of activation, the T cells were stained with WSP-1 dye to quantify intracellular levels of H₂S production in different generations. Interestingly, T cells within the first generation of proliferation (G1) displayed significantly increased levels of intracellular H₂S compared to the naïve T cells (FIG. 12A). However, with progressive T cell proliferation (G2-G4), intracellular H₂S returned to basal levels. Corroborating these findings, mRNA samples collected from activated T cells prior to activation (time 0), and at 24, 48, and 72 hours demonstrated significant upregulation of Cbs (one of the primary enzymes responsible for H₂S production) 24 hours after activation, followed by a return to basal levels of expression by 72 hours (FIG. 12B).

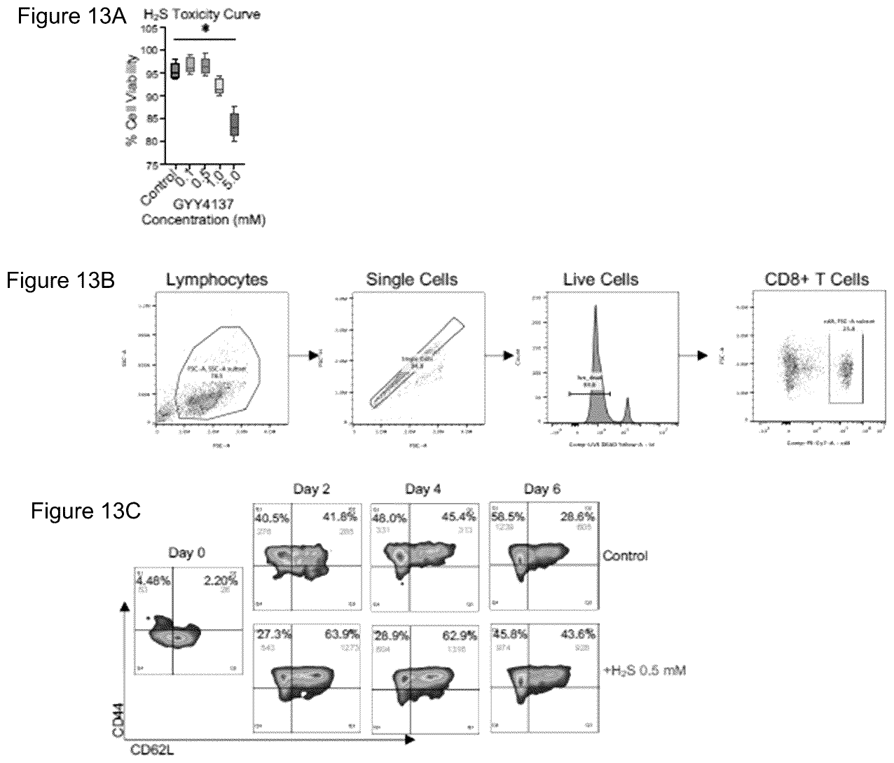

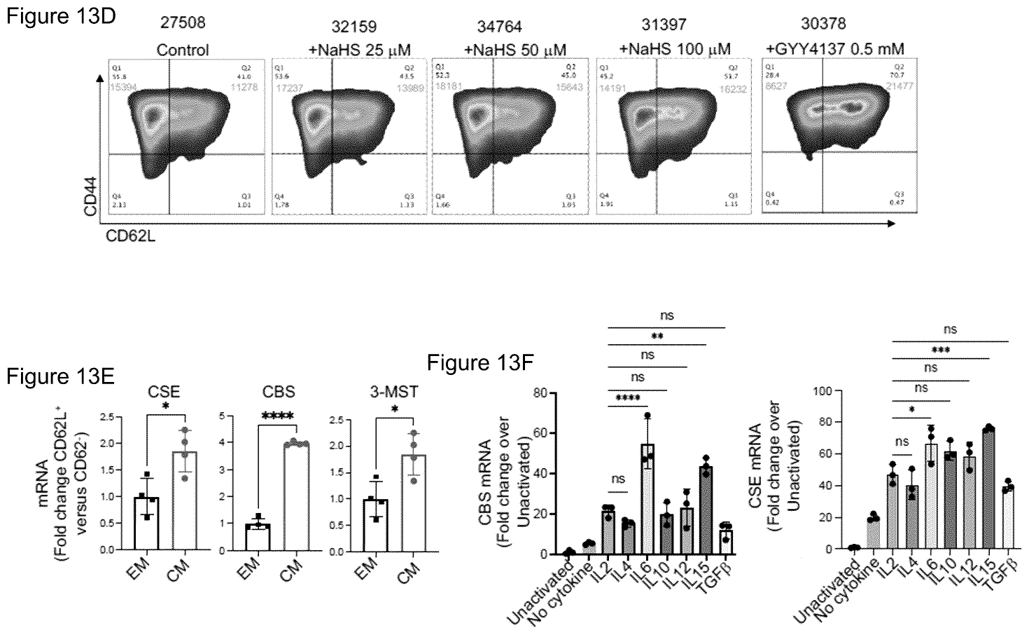

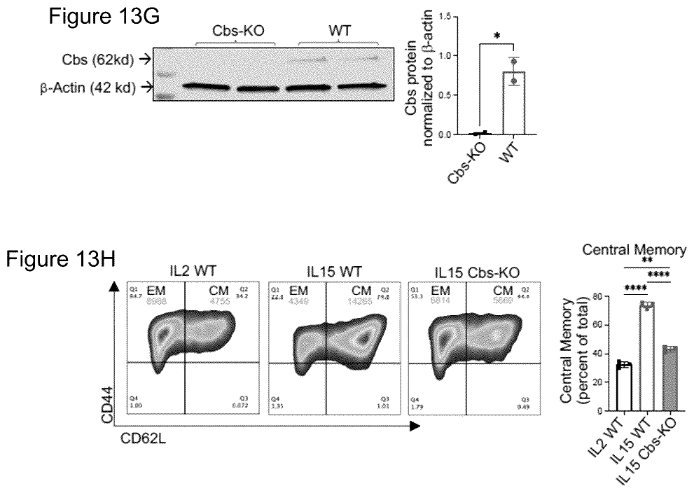

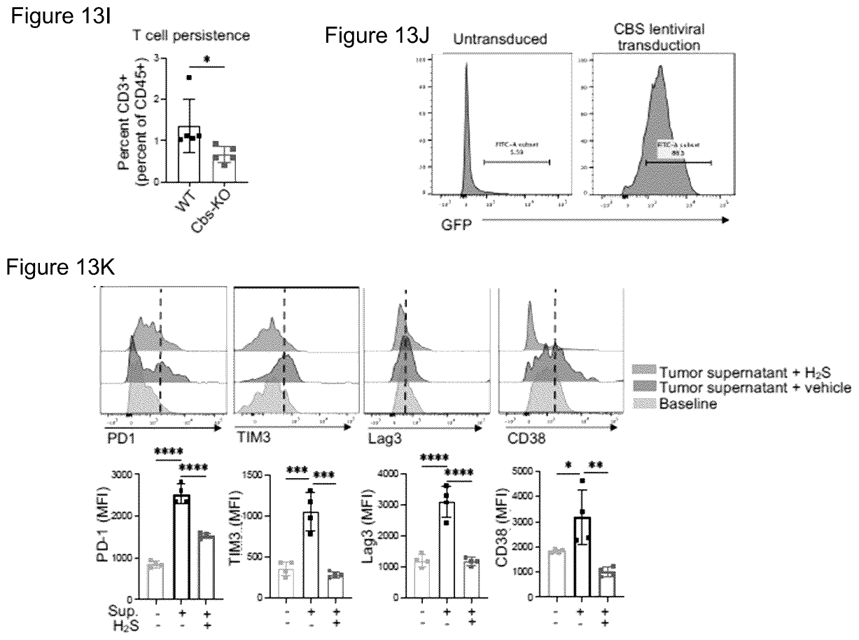

It was next determined if restoring H₂S levels in activated T cells would alter their phenotype. To achieve steady-state levels of H₂S in the T cell culture media, water-soluble H₂S donor GYY4137 that slowly releases sustained levels of H₂S up to 7 days in culture was used (Lee Z. W., et al., 2011, PLOS One, 6(6):e21077). Given the cytotoxic nature of H₂S at high concentrations, 0.5 mM was selected as the optimal dose of GYY4137 (FIG. 13A) in all experiments where Pmel T cells were activated with cognate antigen gp100 for 7 days in presence of rIL2 (50 IU/ml) and gated on the CD8+ fraction for analysis (FIG. 13B). It was observed that T cells generated in presence of H₂S donor had a significant increase in the Tcm phenotype (as determined by CD62L+CD44+ co-expression) compared to those expanded with IL2 alone (FIG. 12C), and this increase in Tcm phenotype was consistently maintained over six days (FIG. 13C). Further analysis revealed that H₂S treatment significantly enhanced the expression of Tcf7 and Sca1, markers related to the T cell stemness phenotype (FIG. 12D). A similar dose-dependent increase in the Tcm phenotype was observed when the fast-release H₂S donor NaHS was used, added to the culture media daily (FIG. 13D), supporting that H₂S plays a role in generation of Tcm phenotype. A qPCR analysis of the activated T cells that were FACS sorted based on effector (CD62L+CD44+) and central memory (CD62L+CD44+) fraction showed increased expression of all three H₂S-producing enzymes (CBS, CSE, 3-MST) in the Tcm fraction, with CBS being most significantly upregulated (FIG. 13E). Interestingly, treatment of T cells with IL15 and IL6, cytokines known to induce the Tcm phenotype in T cells (Schluns K. S., et al., 2002, J Immunol., 168(10):4827-31) and play a key role in memory formation (Nish S. A., et al., 2014, Elife, 3:e01949), respectively, resulted in significantly increased expression of both CBS and CSE. (FIG. 13F).

Importantly, TCR activated T cells from mice lacking Cbs expression (Cbs-KO, FIG. 13G) not only showed reduced H₂S accumulation (FIG. 12E), but also exhibited reduced Tcm fraction when programmed in presence of IL15 (FIG. 13H) and inferior ability to persistent following adoptive transfer compared to wildtype T cells (FIG. 311). However, adding H₂S to the Cbs-KO T cells led to a restoration of the Tcm fraction (FIG. 12F). This data confirms that H₂S levels play an important role in maintaining Tcm phenotype. Importantly, overexpression of Cbs (FIG. 13J) in activated CD8+ T cells also resulted in a significantly enhanced H₂S accumulation (FIG. 12G), and a concomitant increase in the Tcm population and Sca1 expression (FIG. 12H).

The solid tumor microenvironment is characterized as being highly immunosuppressive, leading to T cell exhaustion. Thus, an in vitro model of TME-induced T cell exhaustion using supernatants collected from B16-F10 murine melanoma cells along with chronic antigen stimulation was used.25 Pmel CD8+ T cells were activated and cultured under optimal culture conditions, with tumor supernatant plus repeated stimulation with gp100, or with tumor supernatant plus repeated stimulation with gp100 plus H₂S donor (FIG. 12I). This model was used to determine the impact of T cell exhaustion on Cbs expression and H₂S production by gating Pmel T cells on TIM3+PD1+ (terminally exhausted) and TIM3−PD1− cells. It was found that terminally exhausted T cells expressed significantly less Cbs (FIG. 12J) with less H₂S production (FIG. 12K). Addition of H₂S to the culture not only enabled activated T cells to preserve the Tcm phenotype in the TME exhaustion assay (FIG. 12L), but also decreased expression of T cell exhaustion markers PD1, TIM3, Lag3, and CD38 (FIG. 13K), suggesting a role for H₂S in combating TME-induced T cell exhaustion. This data suggests that endogenous H₂S levels play an important role in regulating T cell effector vs. memory and exhaustion phenotypes.

### H₂S Supports T Cell Effector Function and Protein Translation.

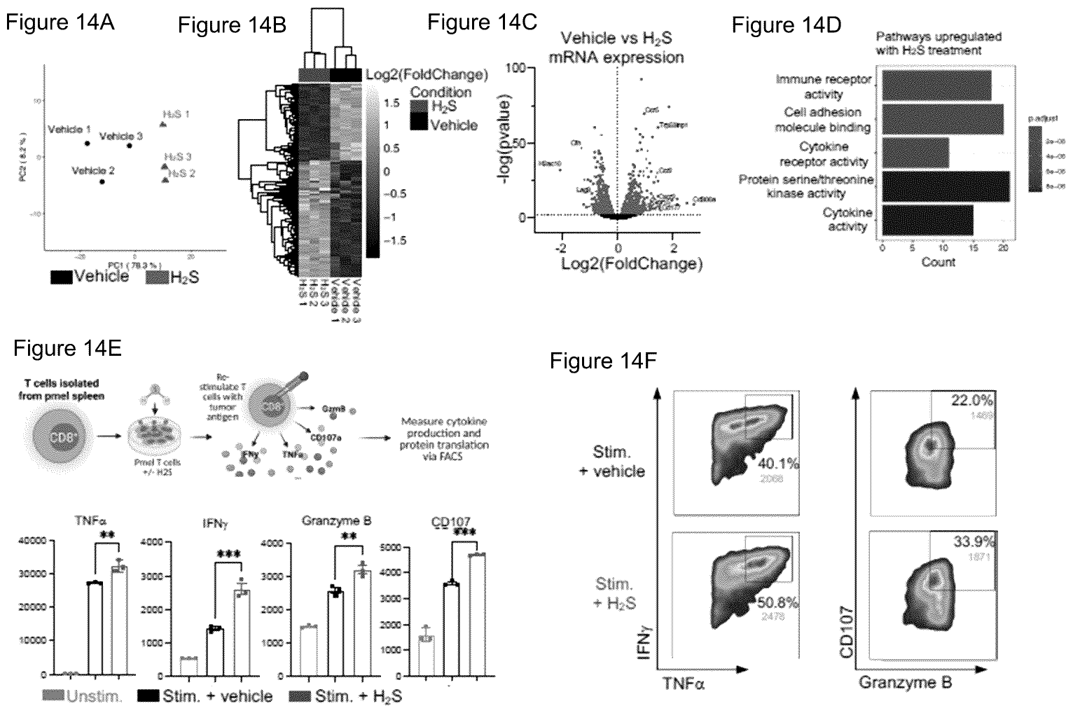

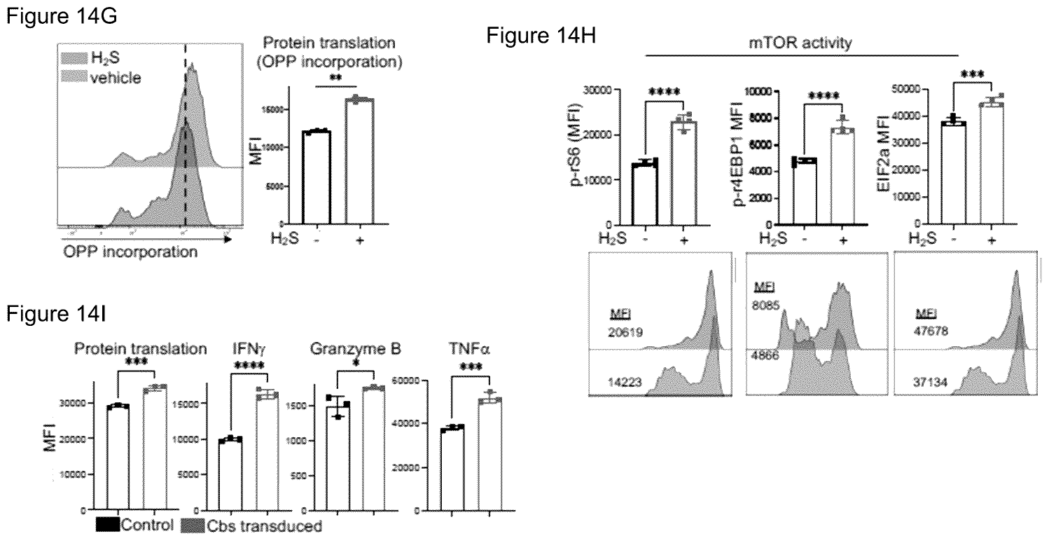

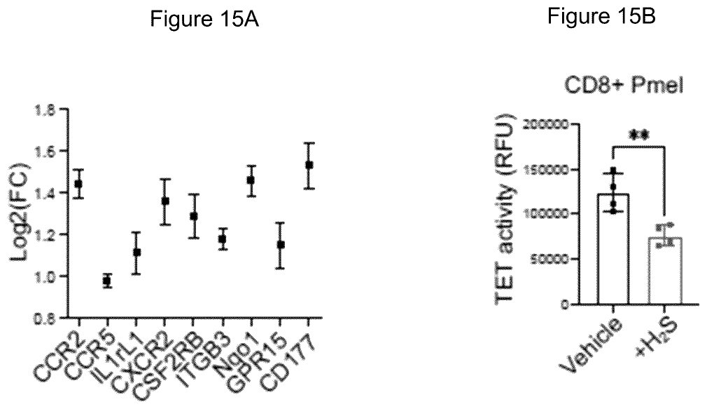

Next, the effect of H₂S treatment on the transcriptomic profile of anti-tumor T cells was determined. Pmel CD8+ T cells were activated and expanded in the presence or absence of the H₂S donor prior to RNA-sequencing. Principle Component Analysis (PCA) demonstrated a distinct transcriptomic profile for T cells treated with the H₂S donor as compared to T cells activated under standard culture conditions (FIG. 14A). Of note, several of the most significantly upregulated genes were genes involved in chemokine receptor activity and leukocyte migration, including CCR2, CCR5, CXCR2, GRP15, and CD177 (FIGS. 14B and 14C). Interestingly, the major pathways significantly upregulated in H₂S-treated T cells included pathways related to immune receptor activity, T cell signaling, and cytokine activity (FIGS. 14C and 14D; FIG. 15A).

The impact of H₂S on T cell effector function was next determined. Pmel CD8+ T cells were activated and expanded with standard culture conditions or with the addition of the H₂S donor and subsequently re-stimulated with gp100 cognate antigen (FIG. 14E). Pmel T cells treated with the H₂S donor displayed enhanced secretion of effector cytokines TNF-α, and IFN-γ, as well as increased levels of cytolytic molecule Granzyme B and CD107 expression (a marker of degranulation) (FIG. 14F) (Aktas E., et al., 2009, Cell Immunol., 254(2):149-54). Given the importance of protein translation in maintaining a robust anti-tumor response and the repression of T cell translation that occurs in the TME (Riesenberg B. P., et al., 2022, bioRxiv, 2022.01.31.478547; Marchingo J. M., et al., 2022, Cell Mol Immunol., 19(3):303-315), it was determined if H₂S influences protein translation. Notably, T cells treated with the H₂S donor demonstrated significantly enhanced upregulation of overall protein translation as measured by O-propargyl-puromycin (OPP) incorporation into the nascent polypeptide chain during translation upon re-stimulation with cognate antigen (FIG. 14G) (Hsu J. C., et al., 2022, STAR Protoc., 3(3):101654). H₂S-treated T cells also displayed increased total levels of EIF2α and phosphorylated levels of S6 ribosomal protein, essential factors regulating the rate-limiting step of protein synthesis, along with increased phosphorylation of 4EBP1 (one of the key negative regulators of protein synthesis which is inhibited by phosphorylation) (FIG. 14H). Similarly, Pmel CD8+ T cells transduced with Cbs overexpressing vector displayed increased cytokine production and overall protein translation when re-exposed to cognate tumor antigen (FIG. 14I), demonstrating that enhancing H₂S signaling increases effector function in CD8+ T cells.

It has previously been shown that in CD4+ T cells, H₂S promotes Tet-mediated Foxp3 demethylation to drive regulatory T cell (Treg differentiation) (Yang R., et al., 2015, Immunity, 43(2):251-63). However, recent studies have shown that reduced Tet2 expression results in improved function of CD19 chimeric antigen receptor engineered T cells (Fraietta J. A., et al., 2018, Nature, 558(7709):307-312). It was observed that in CD8+ T cells, H₂S treatment results in a decrease of Tet activity which also correlated with (Supplementary FIG. 14B). These findings suggest distinct roles of H₂S signaling in CD8+ T cells programming.

### H₂S Enhances Anti-Tumor Efficacy of T Cells In Vivo.

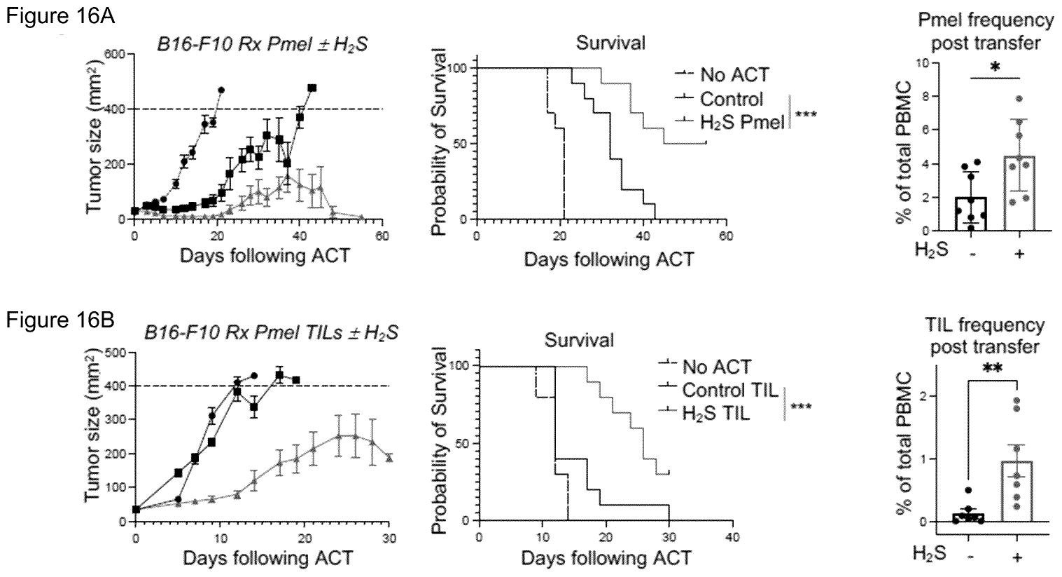

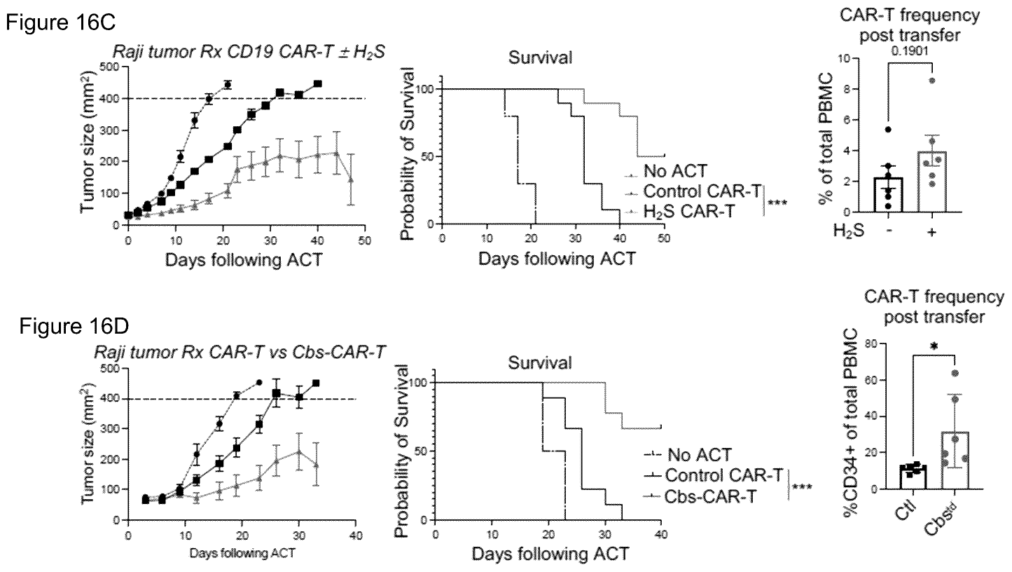

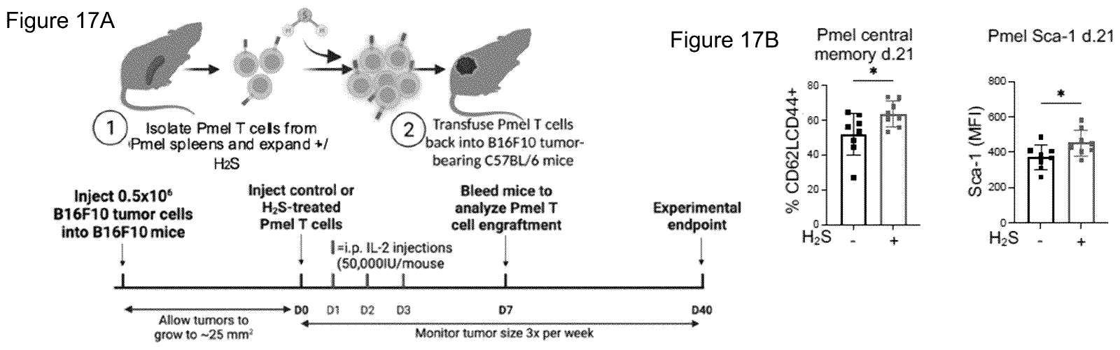

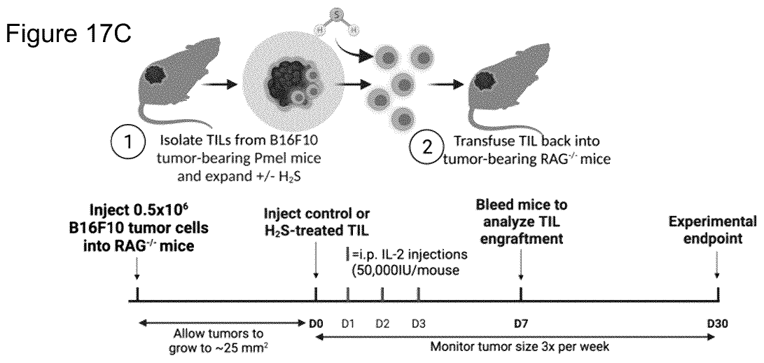

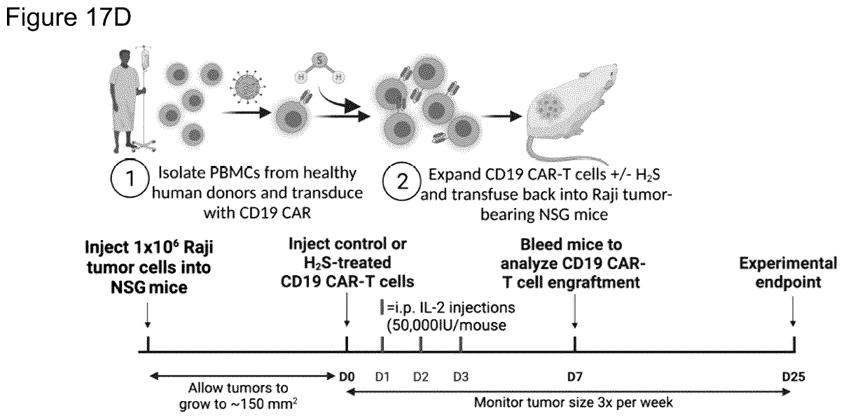

Given the Tcm phenotype, reduced exhaustion, and enhanced effector function of T cells treated with H₂S in vitro, the utility of H₂S-based strategies in tumor control was next assessed. First, Pmel T cells expanded with or without the H₂S donor that were subsequently transferred to B16-F10 murine melanoma bearing immunocompetent C57BL/6 mice were utilized (FIG. 17A, schematic). The H₂S-treated Pmel T cells displayed a superior ability to control tumor growth and prolong overall survival compared to control Pmel T cells (FIG. 16A), which also correlated with increased persistence (right panel). Enhanced CD62L+CD44+ Tcm phenotype and Sca1 expression was also noted in the group that received H₂S pretreated T cells (FIG. 17B). Next, to determine if H₂S treatment would reprogram tumor infiltrating lymphocytes (TILs) and render them more effective, TILs from B16-F10 tumor engrafted in Pmel mice and then expanded them with or without the H₂S donor were obtained (FIG. 17C). Following adoptive transfer into tumor-bearing mice, it was observed that mice receiving H₂S-treated TILs exhibited significant reduction in tumor growth and extended overall survival (FIG. 16B), which also correlated with its enhanced persistence.

Given the demonstrated efficacy of using H₂S to enhance adoptive cell therapy (ACT) protocols in murine models of melanoma, it was determined if this strategy would be equally efficacious in controlling human tumors. Thus, human B cell lymphoma Raji cells engrafted in NSG mice were treated with human CD19 chimeric antigen receptor (CAR) transduced T cells that were either generated in presence or absence of H₂S (FIG. 17D). As expected, mice receiving CD19 CAR-T cells expanded with H₂S had a significant reduction in tumor growth and an increase in overall survival (FIG. 16C), as well as increased persistence (right panel). To further support the translational applicability of these findings, a CAR-T construct to incorporate Cbs was designed. These CD19-Cbs engineered CAR-T cells (Cbstd CAR-T cells) also exhibited better tumor control as compared to the CD19 CAR engineered T cells (FIG. 16D) with increased persistence (right panel). This suggests that anti-tumor T cells with increased H₂S signaling exhibit better persistence in vivo and CAR-T cells engineered to express Cbs can potentiate tumor control.

### H₂S Alters Metabolic Profile and Enhances Mitochondrial Function of T Cells.

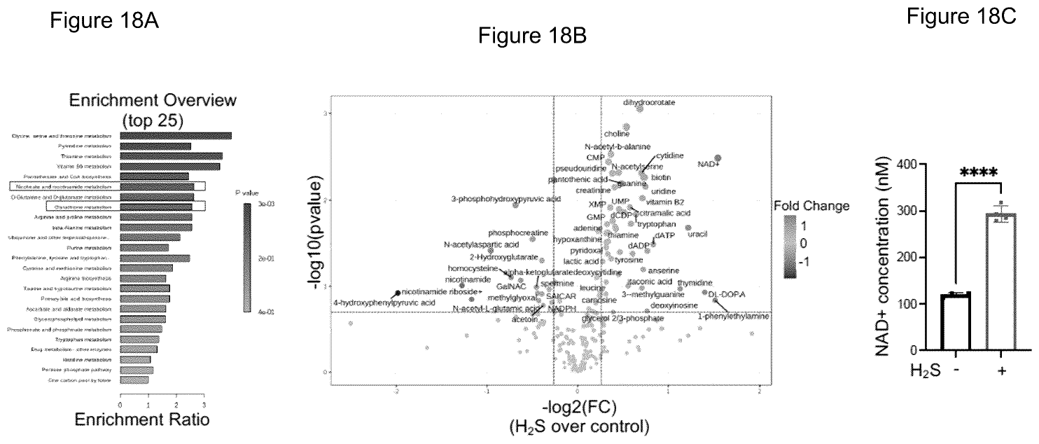

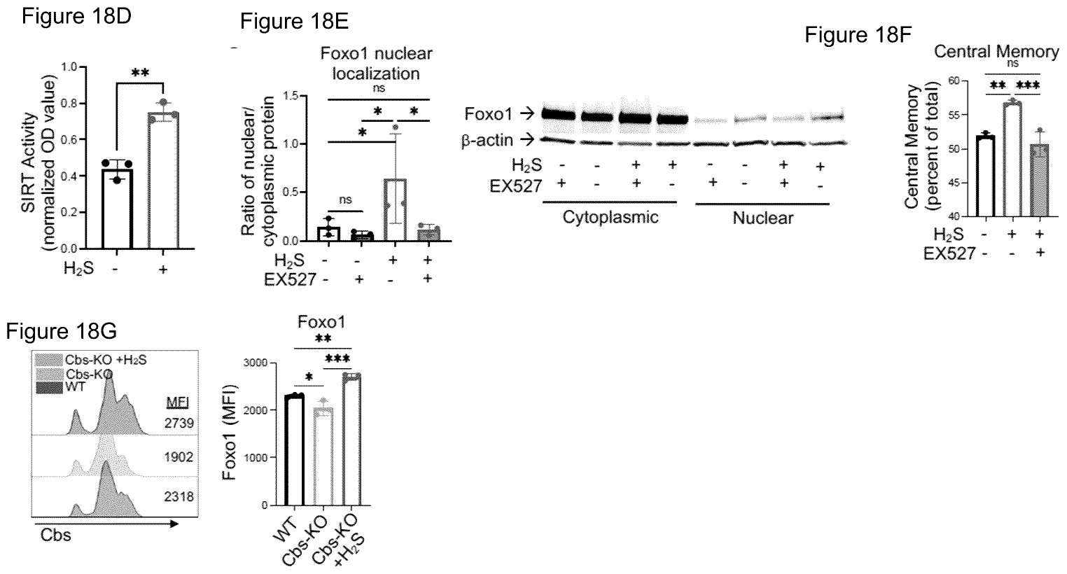

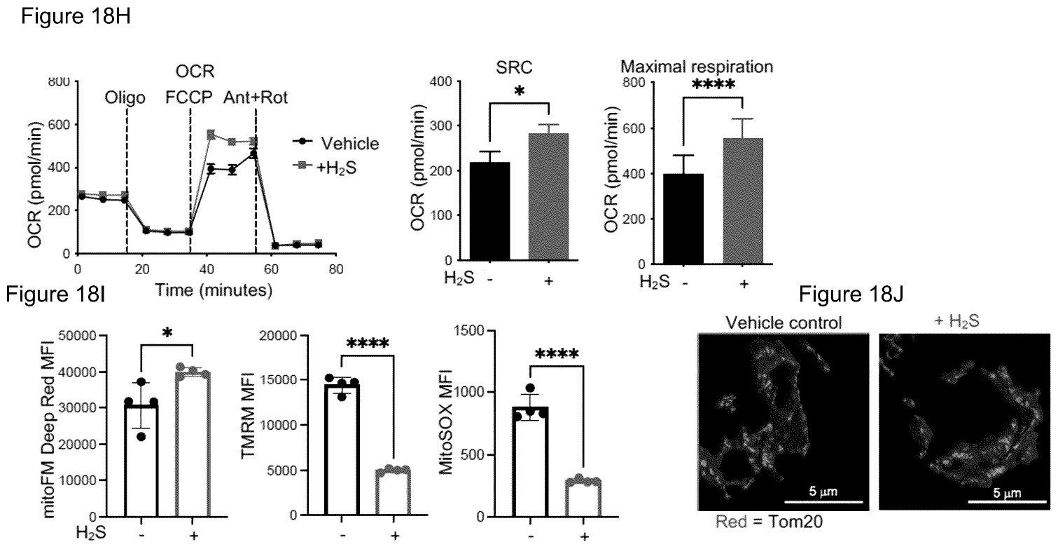

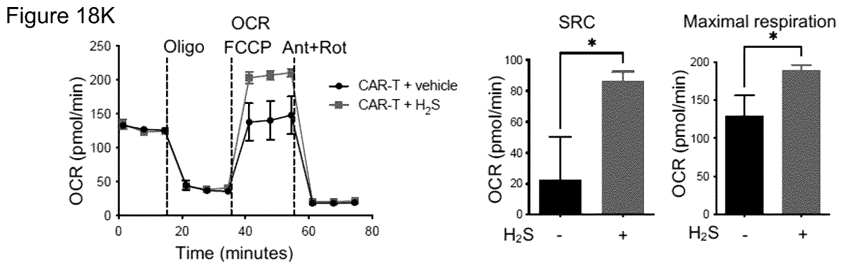

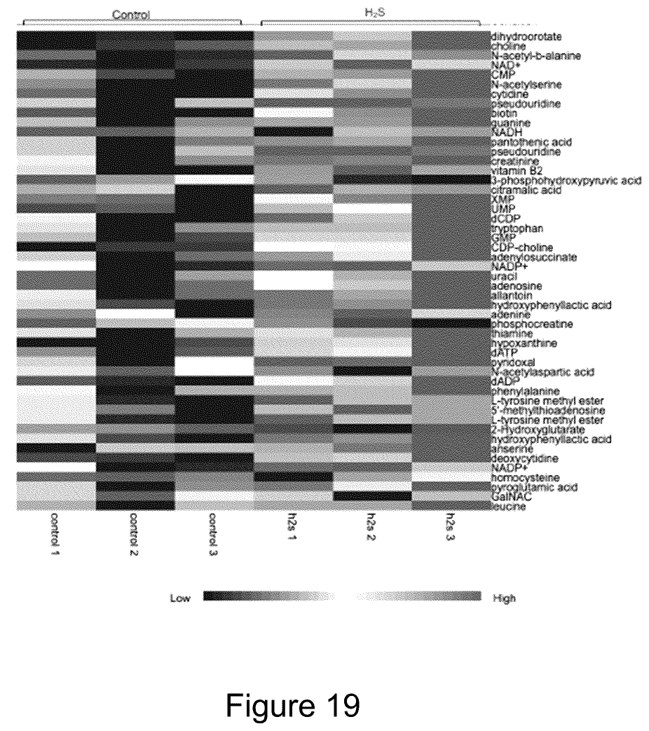

Next, the metabolic status of anti-tumor T cells treated with exogenous H₂S was characterized. Comprehensive metabolomics analysis revealed that Pmel CD8+ T cells treated with the H₂S donor possessed a distinct profile of metabolites relating to enrichment of several metabolic pathways (FIG. 18A; FIG. 19A). Of note, several of the top pathways impacted by H₂S treatment involved metabolic pathways that are known to be critical for anti-tumor immunity, including serine, vitamin B6, and nicotinamide metabolism (FIG. 18A) (Chatterjee S., et al., 2018, Cell Metab., 27(1):85-100.e8; Ma E. H., et al., 2017, Cell Metabolism, 25(2):345-357). Specifically, the pathway of nicotinate and nicotinamide metabolism was enriched, with increased levels of NAD+ and decreased levels of nicotinamide in H₂S-treated T cells (FIGS. 18B and 18C). This corresponded to increased expression of NAD (P) H quinone oxidoreductase 1 (NQO1) identified in the RNA-sequencing analysis, an anti-oxidative enzyme that modulates the differentiation of Th17 cells by regulating ROS levels (Nishida-Tamehiro K., et al., 2022, PLOS One, 17(7):e0272090). This data implies that H₂S renders increased antioxidant capacity to T cells, which may contribute to increase persistence and Tcm phenotype (as reported earlier) (Chakraborty P., et al., 2019, J Biol Chem., 294(23):9198-9212; Kesarwani P., et al., 2014, Cancer Res., 74(21):6036-6047).

Of note, an increase in levels of NAD+ has been linked to enhanced anti-tumor efficacy of CD8+ T cells through its key role as a substrate for Sirt1 (Chatterjee S., et al., 2018, Cell Metab., 27(1):85-100.e8). Indeed, it was found that the H₂S-treated T cells were also characterized by increased Sirt1 activity (FIG. 18D). Previous studies have shown that the transcription factor Foxo1 regulates the Tcm phenotype (Marcel N, Hedrick S. M., et al., 2020, Curr Opin Immunol., 63:51-60), and that NAD+ dependent Sirt1 is responsible for its deacetylation and activity (Chatterjee S., et al., 2018, Cell Metab., 27(1):85-100.e8). Recent studies have also shown that Foxo1 overexpression promotes a stem-like phenotype in CAR-T cells derived from either healthy human donors or patients, which correlates with improved mitochondrial fitness, persistence, and therapeutic efficacy in vivo (Chan J. D., et al., 2024, Nature, 629(8010):201-210; Doan A. E., et al., 2024, Nature, 629(8010):211-218). Thus, whether H₂S treatment increased nuclear translocation of Foxo1 via enhanced Sirt1 activity in T cells was probed. It was observed that H₂S treatment led to increased nuclear localization of Foxo1 and that Sirt1 inhibitor Ex527 treatment neutralized H₂S mediated nuclear localization of Foxo1 (FIG. 18E) and Tcm phenotype (FIG. 18F). Importantly, expression of Foxo1 was reduced in Cbs-KO T cells and was restored upon H₂S treatment (FIG. 18G). This data indicates that the H₂S-mediated increase in Tom phenotype may be dependent on Sirt1-mediated enhancement of nuclear localization of Foxo1.

Next, given the distinct metabolite profile observed in H₂S treated T cells, the mitochondrial fitness of the T cells generated in presence of H₂S was analyzed. Interestingly, it was found that Pmel T cells activated in the presence of the H₂S donor did not alter basal respiration but resulted in a significant increase in both maximal respiration and spare respiratory capacity (SRC) (FIG. 18H). Further, the Pmel T cells treated with the H₂S donor had increased overall mitochondrial mass as measured by mitoFM staining, decreased mitochondrial membrane potential as measured by tetramethylrhodamine methyl ester (TMRM) staining, and decreased mitochondrial reactive oxygen species as measured by mitoSOX staining (FIG. 18I). Notable differences in mitochondrial organization and morphology were also observed with H₂S treatment, including more extended and dispersed mitochondria in the control group compared to more compact and continuous mitochondria in the H₂S treated cells (FIG. 18J). Similarly, the human CD19 CAR-T cells treated with the H₂S donor displayed a significant increase in maximal respiration and SRC (FIG. 18K). Given the previously demonstrated importance of low mitochondrial membrane potential and low mitochondrial reactive oxygen species in metabolically fit anti-tumor T cells with enhanced antioxidant capacity and stemness (Sukumar M., et al., 2016, Cell Metab., 23(1):63-76; Kesarwani P., et al., 2014, Cancer Res., 74(21):6036-6047), these findings suggest an overall increase in mitochondrial fitness in the presence of H₂S.

### H₂S Reduces Oxidative Stress and Golgi-ER Network Stress in Anti-Tumor T Cells.

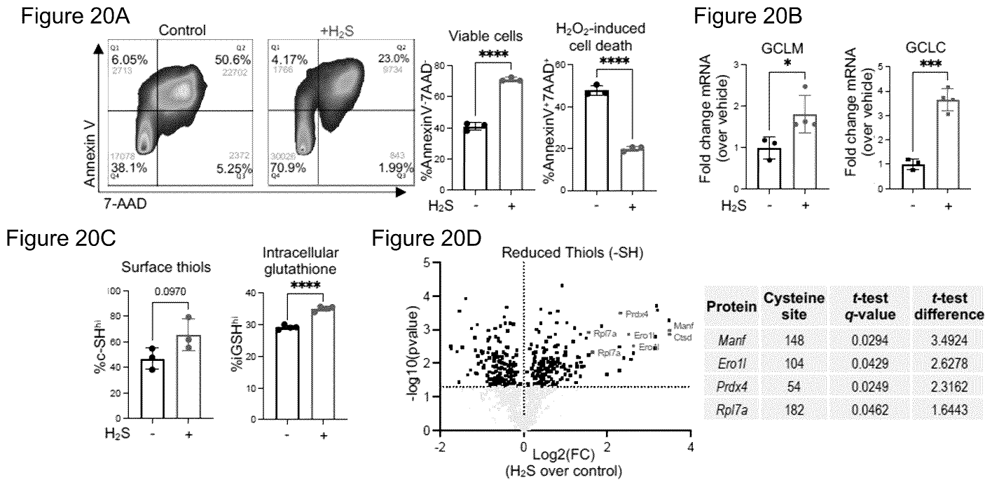

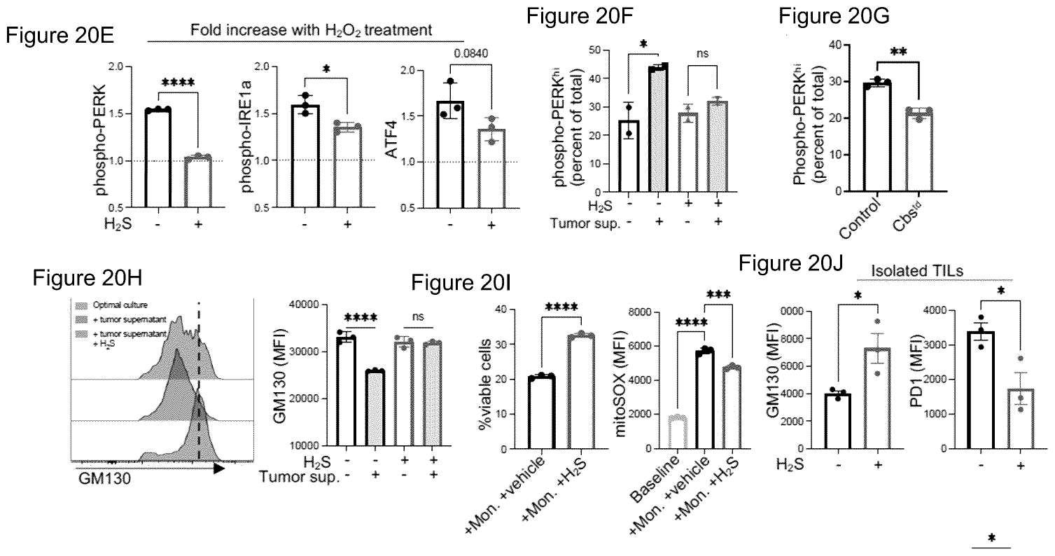

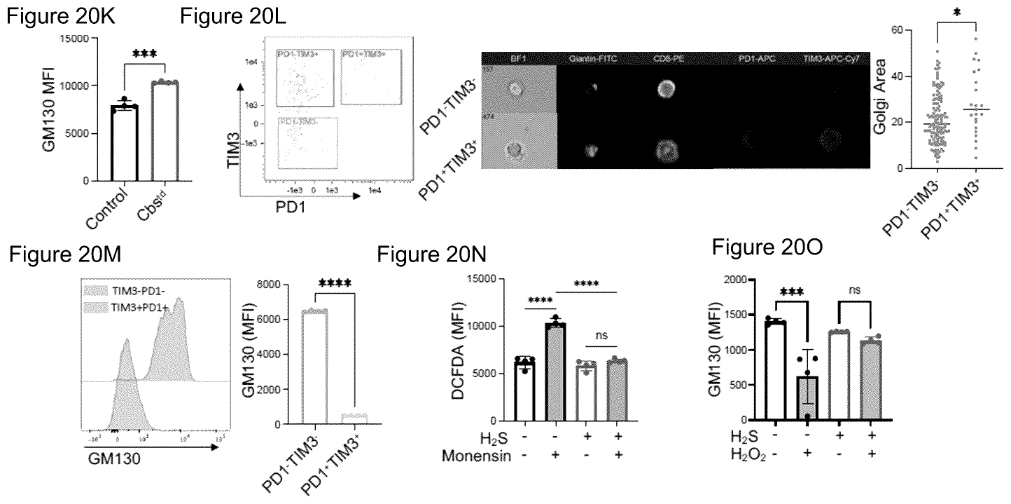

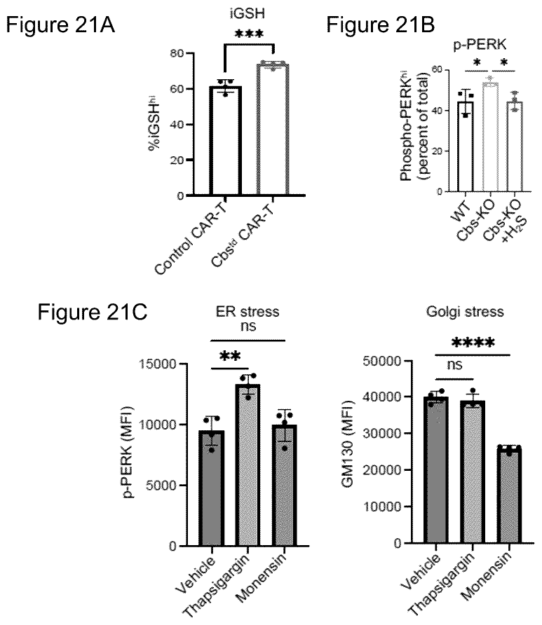

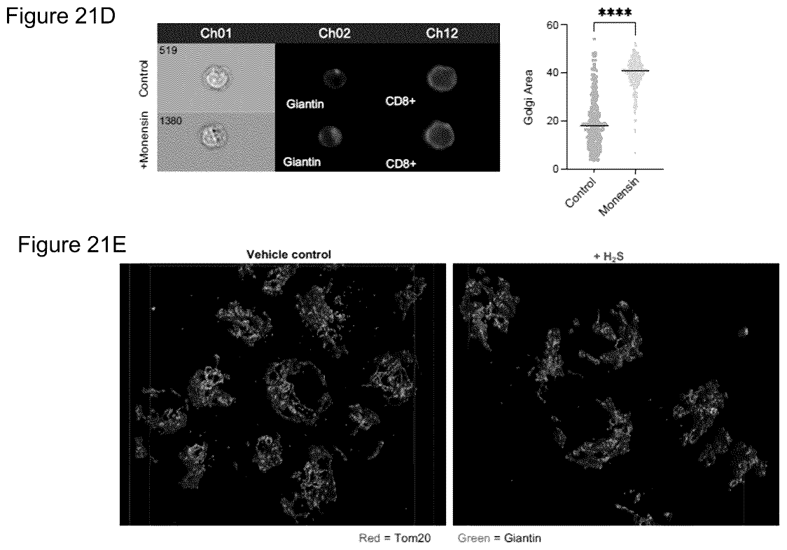

Reducing T cell intrinsic ROS generation has been shown to alleviate T cell exhaustion and improve the efficacy of T cell immunotherapy. Given that H₂S has been identified to upregulate cellular antioxidant defense mechanisms (Lu M., et al., 2008, Free Radic Biol Med., 45(12):1705-13; Kimura Y., et al., 2010, Antioxid Redox Signal., 12(1):1-13; Jain S. K., et al., 2014, Metab Syndr Relat Disord., 12(5):299-302), its ability to combat oxidative stress in anti-tumor T cells was determined. Using hydrogen peroxide (H₂O₂) to induce oxidative stress in vitro, Pmel CD8+ T cells treated with the H₂S donor exhibited a significant decrease in apoptotic cell death (FIG. 20A). H₂S treatment also resulted in increased expression of glutamate cysteine ligase (GCL) holoenzyme catalytic subunit (GCLC) and modifier subunit (GCLM) (FIG. 20B), which catalyzes the rate-limiting step in the formation of the cellular antioxidant glutathione (GSH) to maintain cellular GSH homeostasis. In line with this observation, it was noted that H₂S-treated Pmel T cells and CD19 CAR-T cells overexpressing Cbs exhibited an increase in overall cell-surface thiol (—SH) expression and intracellular glutathione (iGSH) (FIG. 20C; FIG. 21A), correlating to their increased resistance to oxidative stress-induced cell death. Given the observed increase in total surface thiols, a proteomic approach to identify proteins with cysteine thiols sensitive to H₂S treatment was utilized. Proteomics data revealed several key proteins involved in regulating ER stress that display enhanced abundance or an increase in the extent of free thiols in response to H₂S treatment, including the key ER localized proteins Peroxiredoxin-4 (Prdx4), ER oxidoreductin 1 (Ero1), Mesencephalic astrocyte-derived neurotrophic factor 1 (MANF1), and the 60S ribosomal protein L7a (Rp17a) (FIG. 20D).

While Prdx4 supports redox homeostasis by metabolizing H₂O₂ in the ER, its loss leads to oxidative stress and toxicity (Elko E. A., et al., 2021, J Biol Chem., 296:100665). Ero1 is an oxidoreductase enzyme that catalyzes the formation and isomerization of protein disulfide bonds in the ER, generating H₂O₂ in the process (Sevier C. S., et al., 2008, Biochim Biophys Acta., 1783(4):549-56). Given the ER-Golgi intricate association and that Golgi stress response has been shown to reprogram cysteine metabolism (Sbodio J. I., et al., 2018, PNAS, 115(4):780-785), it was hypothesized that H₂S levels regulate oxidative stress within the ER and Golgi network. Indeed, it was observed that markers of ER stress, including phospho-PERK, phospho-IRE1a, and ATF4, were increased when control Pmel T cells were exposed to oxidative stress; however, this increase was mitigated in H₂S-treated T cells (FIG. 20E). The Cbs-KO T cells also exhibited increased ER stress, measured by increased pPERK, which was reduced in presence of H₂S (FIG. 21B). It was also noted a significant increase in ER stress in T cells exposed to the in vitro TME exhaustion model, which was mitigated with H₂S treatment (FIG. 20F). Further, overexpression of Cbs resulted in a decrease in percentage of T cells experiencing high levels of ER stress (phospho-PERKhi) (FIG. 20G).

Golgi stress has recently been identified as an important mediator of redox imbalance in human cells (Alborzinia H., et al., 2018, Communications Biology, 1(1):210), and H₂S signaling has also been shown to be protective against Golgi stress (Zhang Y., et al., 2019, Antioxidants & Redox Signaling, 32(9):583-601). In order to track Golgi stress, the expression of GM130, a Golgi tethered protein that has been shown to control Golgi morphology in response to changes in cellular conditions was determined (Eisenberg-Lerner A., et al., 2020, Nature Communications, 11(1):409). Previous studies have shown that Purkinje neurons in mice that lack the GM130 exhibit Golgi fragmentation and decreased secretory trafficking, leading to ataxia and cell death (Liu C., et al., 2017, PNAS USA, 114(2):346-351). Thus, it was hypothesized that strategies that maintain Golgi homeostasis and avoid Golgi disruption in T cells would also result in increased persistence and preserve effector functions in the TME. Remarkably, induction of T cell exhaustion using the TME exhaustion model produced significant Golgi stress, as measured by a significant decrease in GM130 expression (FIG. 20H). This decrease in GM130 expression was mitigated in the presence of the H₂S donor (FIG. 20H). To further determine the impact of Golgi stress on T cells, a known Golgi stressor, monensin, was utilized to induce Golgi stress in Pmel T cells in the presence or absence of H₂S. Monensin treatment induced cell death and cellular accumulation of mitochondrial ROS, which was partially mitigated with H₂S treatment (FIG. 20I). TILs isolated from B16-F10 tumors and expanded under normal control conditions (without H₂S) or with H₂S revealed significantly higher GM130 expression and lower PD1 expression in the H₂S treated group (FIG. 20J). These results demonstrate that Golgi stress is induced in the TME and that H₂S can be used to reprogram TILs to reduce Golgi stress, correlating to a decrease in T cell exhaustion. Similarly, Pmel T cells transduced with Cbs and adoptively transferred into tumor-bearing mice maintained significantly higher GM130 expression when isolated from the tumors (FIG. 20K). Importantly, monensin did not induce ER stress, nor did Thapsigargin induced Golgi stress (FIG. 21C), highlighting the exclusivity of organelle stress and need for mitigating them comprehensively to obtain a “stress-free” T cell.

Golgi dispersion under conditions of stress has been identified as a key feature of Golgi dysfunction (Eisenberg-Lerner A., et al., 2020, Nature Communications, 11(1):409). Thus, Golgi stress was induced with monensin and characterized Golgi dispersion with ImageStream analysis, showing that Golgi dispersion significantly increased when T cells were treated with monensin (FIG. 21D). Next, T cell exhaustion was induced using the previously described in vitro TME model and characterized Golgi dispersion in terminally exhausted T cells (PD1+ TIM3+) compared to healthy T cells (PD1−TIM3−). Like monensin treatment, a significant increase in Golgi dispersion in PD1+ TIM3+ T cells compared to PD1−TIM3− was observed (FIG. 20L). This correlated with the observation that PD1+ TIM3+terminally exhausted T cells had significantly lower expression of GM130 compared to PD1−TIM3− T cells (FIG. 20M). Given the observation that H₂S treatment protects anti-tumor T cells from oxidative stress, it was determined whether oxidative stress plays a role in mediating Golgi stress. Interestingly, when Pmel T cells were treated with Monensin to induced Golgi stress, a significant increase in ROS (DCFDA) was observed, which was mitigated with H₂S treatment (FIG. 20N). Importantly, the changes in Golgi stress (quantified by GM130 levels) mediated by oxidative stress (using H₂O₂) were mitigated by H₂S (FIG. 20O). Further, human T cells were also evaluated for Golgi dispersion by confocal microscopy, along with mitochondria staining to establish if there is any spatial relationship between the Golgi and mitochondria. Compared to vehicle control, H₂S-treated T cells had more compact Golgi organization. Interestingly, a close spatial relationship between the Golgi and mitochondria with areas of overlap between the two organelles was also observed (FIG. 21E).

### Prdx4 Regulates H₂S-Mediated Inhibition of Golgi Stress.

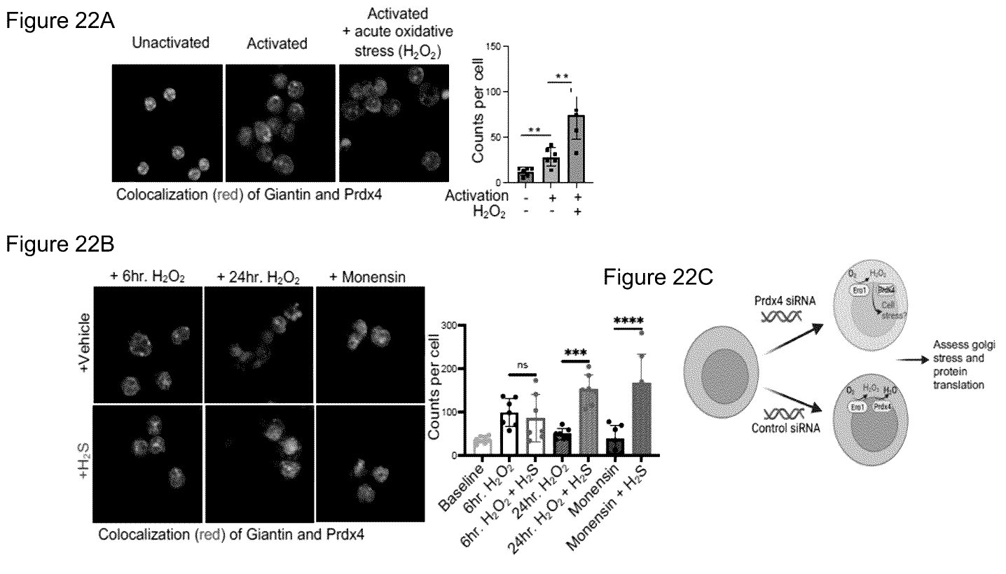

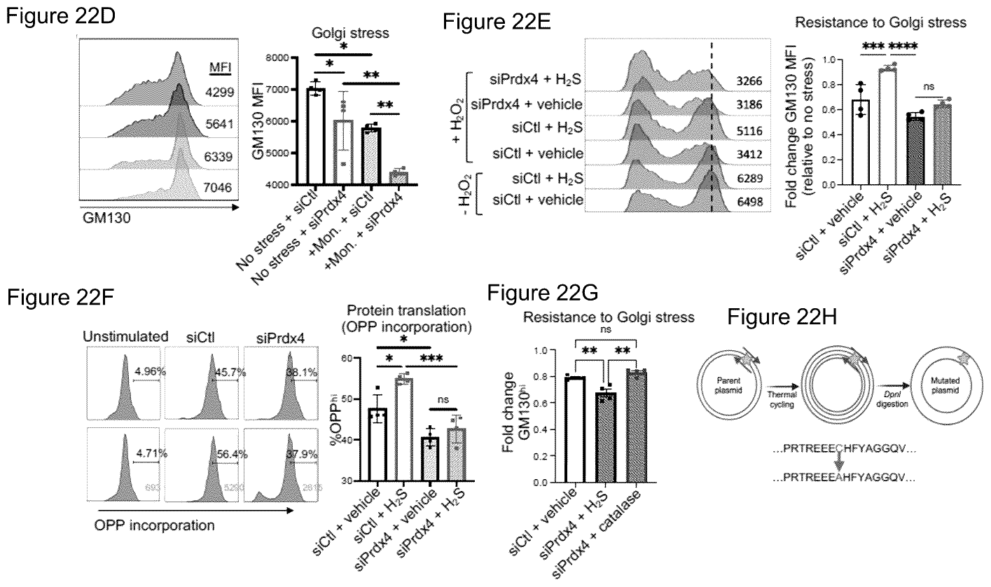

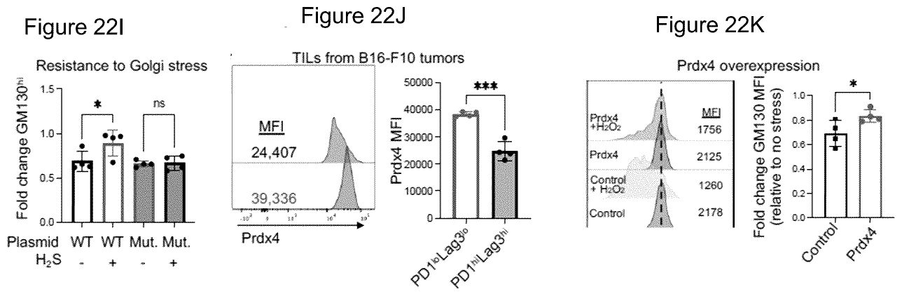

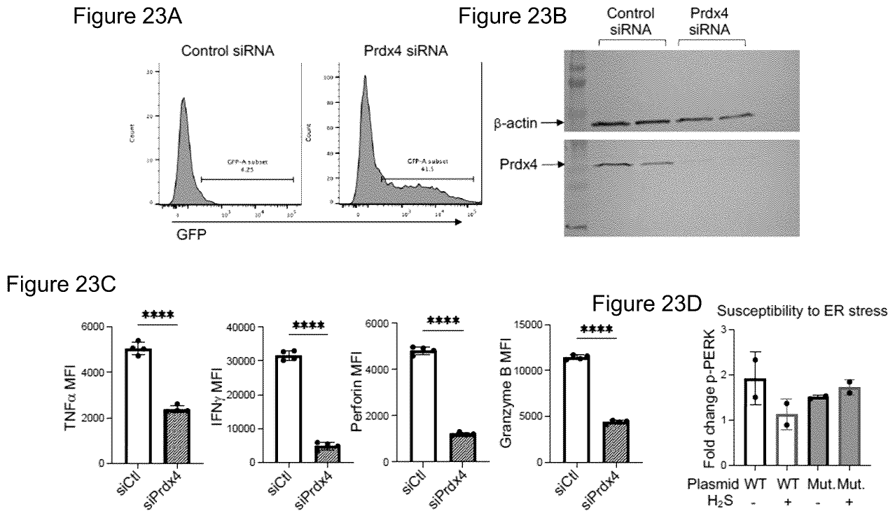

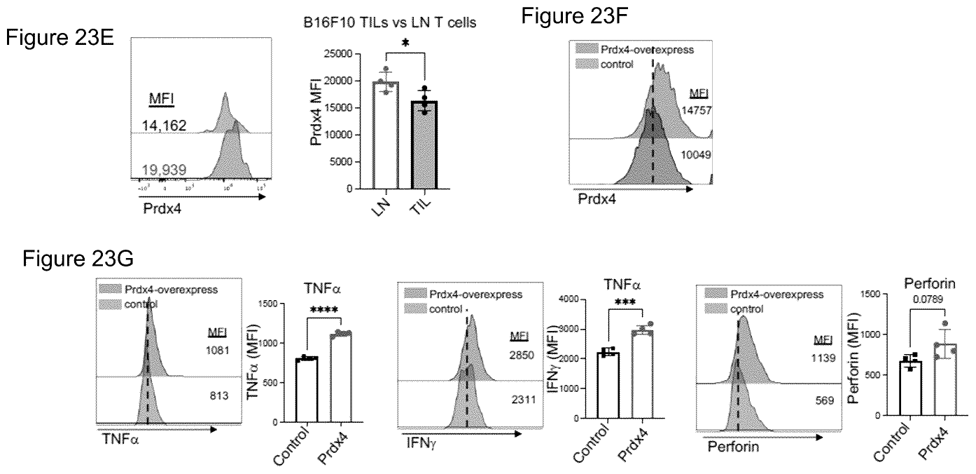

The proteomics screen (FIG. 20D) suggested that an increase in free thiols at cysteine 54 in Prdx4 is promoted by H₂S treatment Importantly, Prdx4 is a key peroxiredoxin enzyme that is involved in regulating redox balance and oxidative stress within the ER. Given the delicate redox balance necessary for the native disulfide bonds, it was postulated that a localized mechanism for the detection and elimination of ROS produced during the oxidative folding process may require Prdx4. Thus, it was hypothesized that ER and Golgi network stress mitigated by H₂S is mediated through the Prdx4 localization and activity in these organelles. First, it was established if Prdx4 also localizes within the Golgi apparatus in T cells using a proximity ligation assay (PLA), a tool that allows in situ detection of endogenous proteins with high specificity and sensitivity, with antibodies directed against Giantin (a conserved Golgi membrane protein) and Prdx4. Interestingly, it was observed that Prdx4 localized within the Golgi apparatus upon T cell activation (FIG. 22A). This localization was further increased upon induction of acute oxidative stress, suggesting a novel role of Prdx4 in responding to oxidative stress within the Golgi (FIG. 22A). Prolonged oxidative stress ultimately resulted in loss of loss of Prdx4 localization within the Golgi, resembling a colocalization profile similar to cells treated with monensin to induce Golgi stress (FIG. 22B). Notably, this loss of colocalization was mitigated with H₂S treatment (FIG. 22B). Next, using siRNA targeting Prdx4, expression of Prdx4 in CD8+ Pmel T cells was knocked down (FIG. 22C, FIGS. 23A and 23B). The cells were then subjected to conditions of oxidative and Golgi stress in the presence or absence of H₂S treatment. Of note, knockdown of Prdx4 resulted in a significant decrease in GM130 expression (FIG. 22D). This decrease was further exacerbated with monensin treatment, demonstrating an increase in susceptibility to Golgi stress in the absence of Prdx4 (FIG. 22D). Then, to determine whether the protective effect of H₂S on Golgi stress is dependent on the presence of Prdx4, Golgi stress was induced in the presence or absence of H₂S with control (siCtl) and Prdx4 knockdown (siPrdx4) T cells. Of note, while H₂S prevented the decrease in GM130 expression in the control T cells, this protective effect was absent in the Prdx4 knockdown T cells (FIG. 22E). A similar dependence on the presence of Prdx4 was observed for the effect of H₂S on increasing protein translation, which was not observed upon H₂S treatment in the Prdx4 knockdown T cells (FIG. 22F). In accordance, the Prdx4 silenced T cells showed reduced production of effector cytokines (IFNγ, TNFα) and cytolytic molecules (granzyme B and perforin) (FIG. 23C).

Given the key role of Prdx4 in scavenging superoxide species and the previous findings indicating a role for oxidative stress (specifically H₂O₂) in disrupting the Golgi, it was hypothesized that it is an organelle specific H₂O₂ scavenging function of Prdx4 which is critical for its ability to protect against Golgi stress. To test this, Golgi stress was induced in control or Prdx4 knockdown T cells in the presence of H₂S or the direct H₂O₂ scavenger catalase. As observed previously, the protective effect of H₂S in mitigating Golgi stress was absent when Prdx4 was knocked down, but protection against Golgi stress could be rescued with the addition of catalase (FIG. 22G). Given the increase in free thiol at cysteine 54 in Prdx4 with H₂S, Prdx4 knockout Jurkat cells were transfected with plasmids containing either wildtype Prdx4 or a version of Prdx4 mutated at the homologous human residue, C51A (FIG. 22H). Interestingly, the protective effect of H₂S on reducing H₂O₂-induced Golgi stress was only observed in Jurkat cells expressing wildtype Prdx4 and not in the cells expressing the cysteine-mutated form of the protein (FIG. 22I). A similar Prdx4-dependent effect of H₂S on reducing ER stress was observed Jurkat cells expressing either the normal or cysteine-mutated form of Prdx4 (FIG. 23D).

To further establish the physiological relevance of T cell Prdx4 expression in the TME in vivo, TILs were isolated from B16-F10 tumors and assessed levels of Pdrx4 in PD1^{hi}Lag3^{hi} terminally exhausted TILs vs PD1^{lo}Lag3^{lo} TILs. Notably, a significant decrease in Prdx4 expression in PD1^{hi}Lag3^{hi} terminally exhausted TILs was observed (FIG. 22J). Additionally, a significant decreased in Prdx4 expression in antigen-experienced TILs compared to antigen-experienced T cells in the tumor draining LNs was observed, suggesting a suppressive effect of the TME on sustained Prdx4 expression (FIG. 23E). To further verify the role of Prdx4 in sustaining T cell function and mitigating Golgi stress, lentivirus supernatant was generated to overexpress Prdx4 in activated Pmel T cells (FIG. 23F). Pmel T cells overexpressing Prdx4 were more resistant to loss of GM130 compared to control Pmel T cells (FIG. 22K). Upon restimulation, Pmel T cells overexpressing Prdx4 produced more cytolytic cytokines compared to control Pmel T cells (FIG. 23G). Collectively, these findings demonstrate a protective effect of H₂S in reducing Golgi stress that is at least partially dependent on the H₂O₂-scavaging capacity of Prdx4.

### Golgi^{hi} Cells Exhibit Enhanced Functionality and Stem-Like Features.

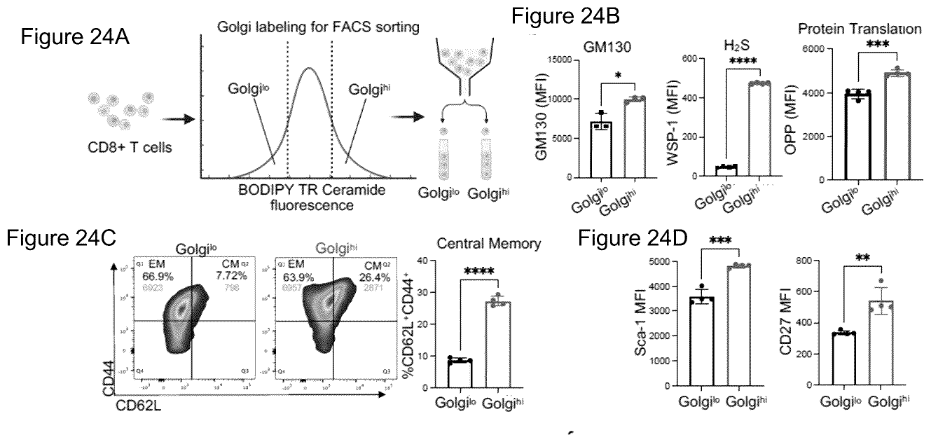

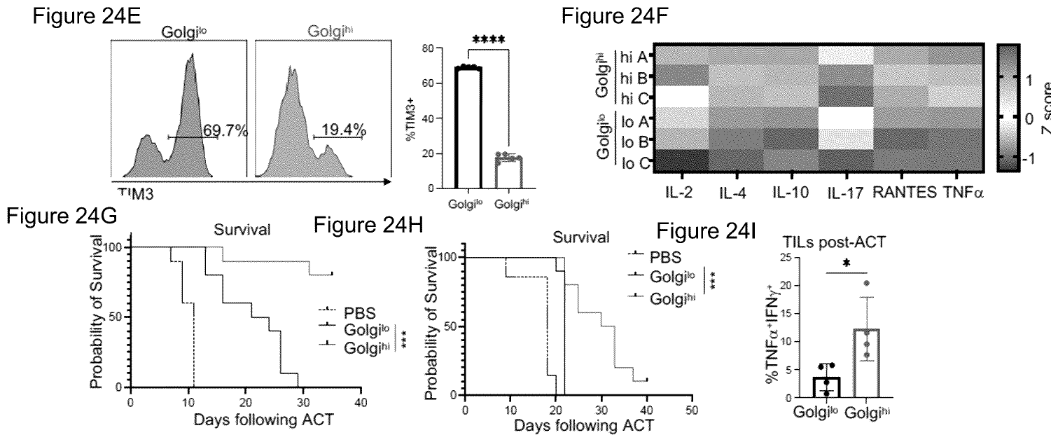

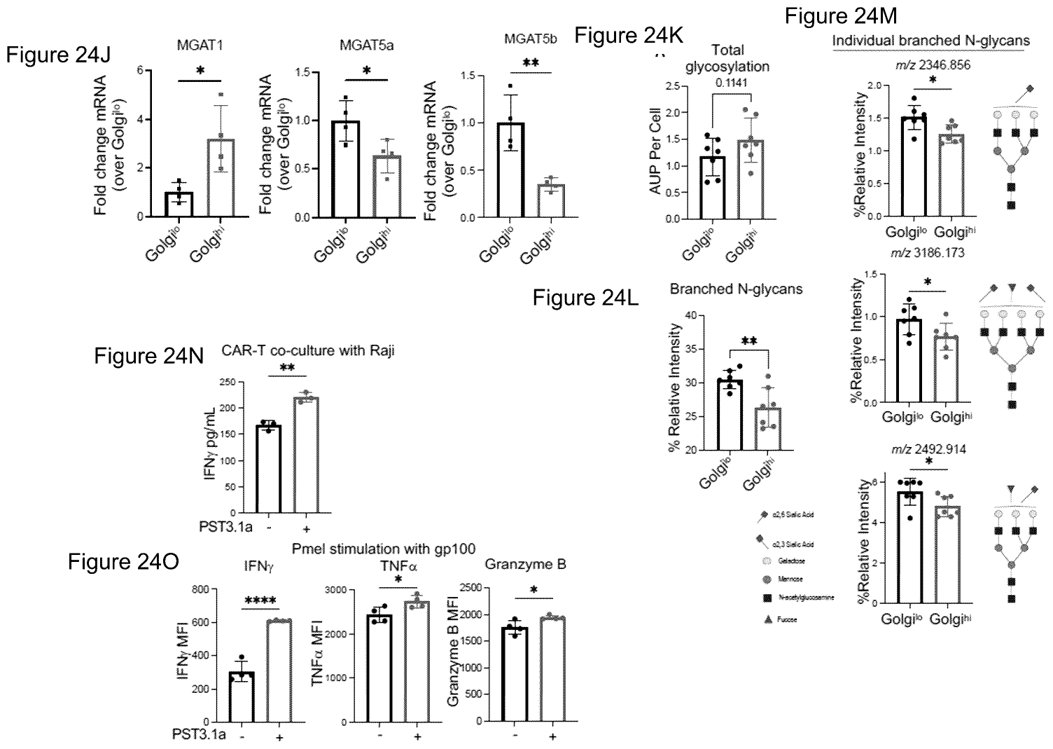

Based on the findings that T cells exposed to the TME exhibit increased Golgi stress, which correlated with a decrease in GM130 levels and a decrease in anti-tumor function, it was determined if Golgi content itself would correlate with T cell anti-tumor function. Thus, activated CD8+ T cells were stained using a fluorescent dye to label the Golgi and were subsequently sorted into cells possessing high Golgi content (Golgi^{hi}) and low Golgi content (Golgi^{lo}) (FIG. 24A). Significantly increased levels of GM130, intracellular H₂S, and protein translation in the Golgi^{hi} T cells upon re-stimulation with gp100 tumor antigen compared to the Golgi^{lo} T cells was observed (FIG. 24B). The Golgi^{hi} subset also exhibited an increase in the Tcm phenotype, with Golgi^{hi} cells having a higher proportion of CD62L+CD44+ cells (FIG. 24C) and higher expression of Sca-1 and CD27 (FIG. 24D).

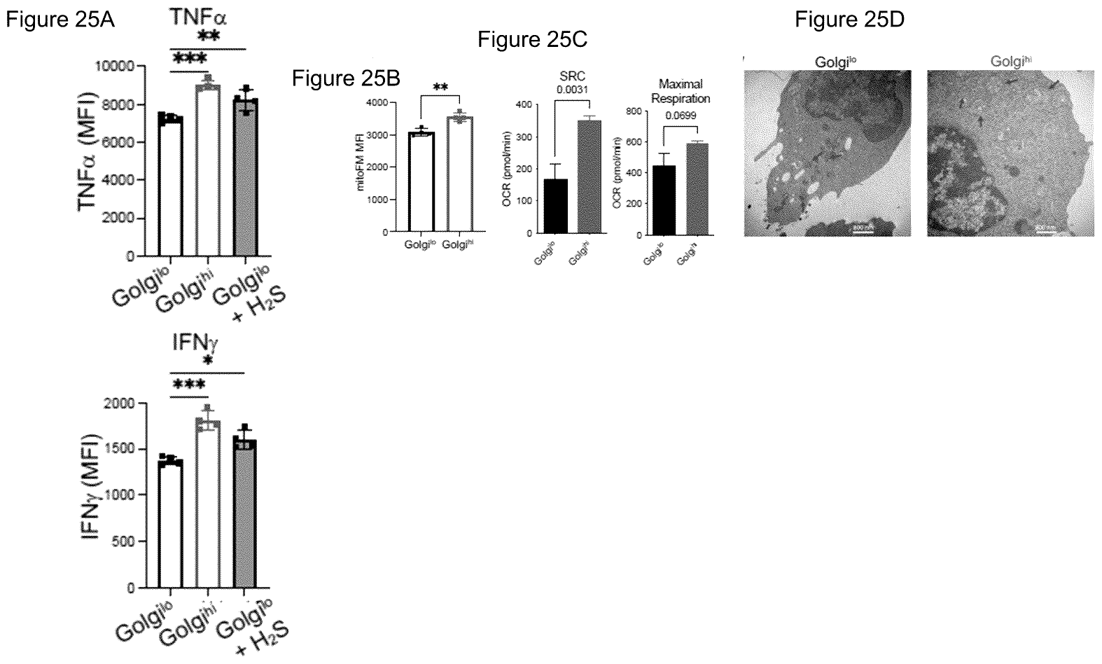

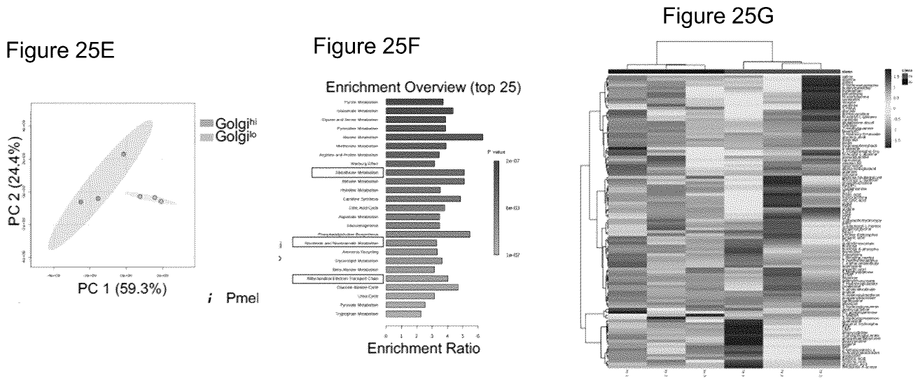

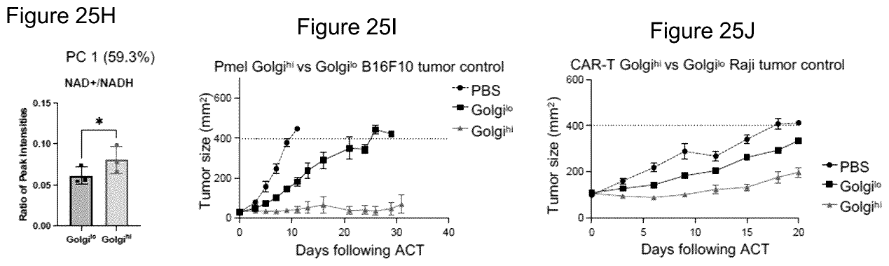

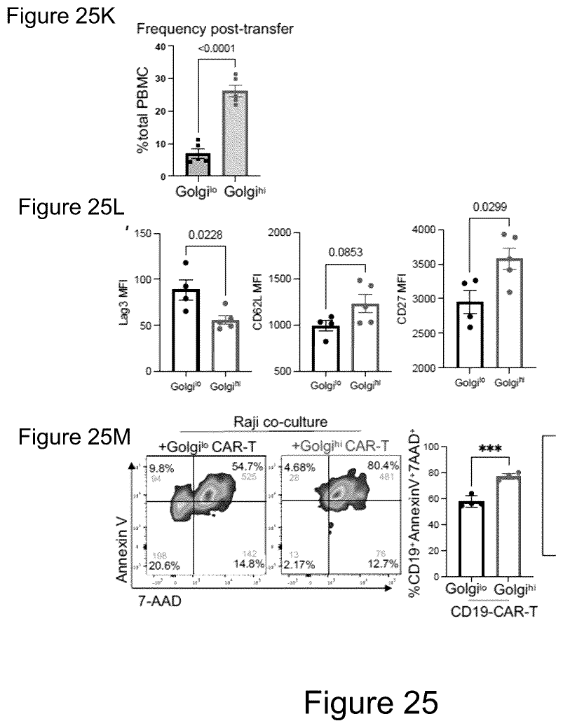

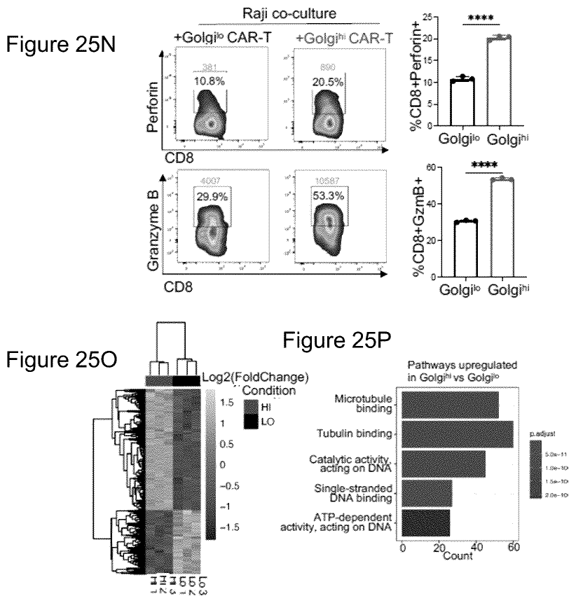

In the TME exhaustion assay, Golgi^{lo} cells had a significantly higher expression of TIM3 as compared to Golgi^{hi} cells, indicating a resistance to T cell exhaustion in the Golgi^{hi} subset (FIG. 24E). Functionally, Golgi^{hi} cells secreted more pro-inflammatory cytokines upon restimulation with cognate antigen, including IL-2, IL-4, RANTES, and TNF (FIG. 24F). Interestingly, it was observed that treating Golgi^{lo} T cells with H₂S resulted in partial restoration of their effector functions (FIG. 25A). Golgi^{hi} cells also were also characterized by a significant increase in mitochondrial mass compared to Golgi^{lo} (FIG. 25B). Similar to H₂S-treated T cells, Golgi^{hi} T cells also displayed a significant increase in spare respiratory capacity, demonstrating a correlation between Golgi^{hi} cells and enhanced mitochondrial fitness (FIG. 25C). Transmission electron microscopy (TEM) analysis of Golgi^{hi} versus Golgi^{lo} cells revealed large, healthy-appearing mitochondria in Golgi^{hi} cells compared to small, dense mitochondria in the Golgi^{lo} cells (FIG. 25D). Metabolomics analysis revealed significant differences in the metabolite profiles of Golgi^{hi} versus Golgi^{lo} cell (FIG. 25E). Of note, several key metabolic pathways were found to be upregulated in the Golgi^{hi} cells compared to Golgi^{lo} cells, including glutathione metabolism, nicotinate/nicotinamide metabolism, and mitochondrial electron transport train (FIGS. 25F and 25G). Similar to H₂S-treated T cells, Golgi^{hi} cells had a significantly higher NAD+/NADH ratio (FIG. 25H).

The anti-tumor capacity of Golgi^{hi} versus Golgi^{lo} cells was next determined, hypothesizing that Golgi^{hi} cells would exert superior tumor control when adoptively transferred into tumor-bearing hosts. Indeed, it was found that Golgi^{hi} Pmel T cells displayed significantly better tumor control (FIG. 25I) and improved survival (FIG. 24G) in B16-F10 bearing C57BL/6 mice. Additionally, when human CD19 CAR-T cells were sorted into Golgi^{hi} and Golgi^{lo} populations and adoptively transferred to the NSG mice engrafted with Raji tumor cells, the Golgi^{hi} CAR-T cells displayed superior control of lymphoma (FIG. 25J) and prolonged host survival (FIG. 24H). Adoptively transferred Golgi^{hi} cell were detected at higher circulating frequencies post-transfer (FIG. 25K), exhibited reduced exhaustion and preserved Tcm phenotype (LAG3^{lo}CD62^{hi}CD27^{hi}) (FIG. 25L), and maintained superior effector function when isolated from the TME (FIG. 24I). When co-cultured with Raji cells, Golgi^{hi} CAR-T cells also induced significantly more tumor cell death compared to the Golgi^{lo} CAR-T cells (FIG. 25M) and produced more perforin and granzyme B (FIG. 25N). These data confirm that sorting T cells based on the status of the Golgi apparatus identifies T cells with superior ability to control tumor cells.

To further identify the pathways that define the Golgi^{hi} vs. Golgi^{lo} subsets, RNA sequencing analysis was performed on human CD19 CAR-T cells, which revealed a district transcriptomic profile between the two subsets (FIG. 25O). Fascinatingly, the two top-upregulated pathways in the Golgi^{hi} subset involved microtubule and tubulin binding (FIG. 25P). Of note, previous studies have identified a critical role for Golgi proteins in coordinating with the microtubule organizing center (MTOC) to facilitate transportation of key signaling molecules to the immunological synapse (Zucchetti A. E et al., 2019, Nature Communications, 10(1):2864). Additionally, RNA sequencing analysis revealed significant differences in levels of key enzymes involved in regulating N-glycan branching in the Golgi, including an increase in MGAT1 (1,6 N-acetylglucosaminyltransferase I, a negative regulator of N-glycan branching) and a decrease in MGAT5A/B (β1,6 N-acetylglucosaminyltransferase Va/Vb, the rate-limiting enzyme in N-glycan branching), which were validated by RT-PCR (FIG. 24J). Given the critical role of the Golgi in post-translationally modifying proteins via glycosylation, the N-linked glycosylation on Golgi^{hi} vs Golgi^{lo} T cells was profiled using a method published by Dressman et al. (Dressman J. W., et al., 2023, Analytical Chemistry, 95(27):10289-10297). While an overall increase in total glycosylation in Golgi^{hi} cells was observed, it was not statistically significant (FIG. 24K). However, a notable decrease in overall branched N-glycans was observed (FIGS. 24L and 24M), which correlated with the reduced expression of Mgat5a/b. Levels of branched N-glycans have been shown to directly correlated with the T-cell activation threshold in an Mgat5-dependent manner, while Mgat1 has been shown to paradoxically inhibit activity of Mgat5 leading to decreased N-glycan branching (Smith L. K., et al., 2018, Immunity, 48(2):299-312.e5; Demetriou M., et al., 2001, Nature, 409(6821):733-9; Chen H. L., et al., 2009, J Biol Chem., 284(47):32454-61). To further validate the importance of decreased Mgat5 activity and thus low N-glycan branching in the Golgi^{hi} subset, activated CD8+ T cells were treated with the Mgat5 inhibitor Phostine PST3.1a, a selective inhibitor of Mgat5 enzymatic activity which has been shown to have anti-tumor activity in in vivo models of glioblastoma (Hassani Z., et al., 2017, Molecular Cancer Research, 15(10):1376-1387). As predicted, inhibition of Mgat5 activity in both human and murine T cells resulted in a more potent effector response upon encountering tumor antigen, as evidenced by an increase in IFNγ production by CD19 CAR-T cells when co-cultured with Raji tumor cells (FIG. 24N) and an increase in cytolytic cytokine production by Pmel T cells upon re-stimulation with gp100 peptide (FIG. 24O). Thus, a role for the Golgi in modulating protein glycosylation and rendering a robust anti-tumor phenotype to T cells could be a key factor in determining immunotherapeutic outcomes.

### Discussion

The cumulative role of cellular organelles in shaping the life and function of a cell has been long acknowledged (Zhang Y., et al., 2023, Cell Death Discov., 9(1):51). While each organelle plays a specific role in the growth and development of T cells, numerous studies have thus far focused on targeting mitochondria, endoplasmic reticulum (ER), or lysosome related pathways to improve the anti-tumor T cell immune response. Strategies mitigating stress in these organelles have shown to improve T cell fitness and enhance tumor control. Increasing evidence suggests that the Golgi apparatus also plays a crucial function in sensing and integrating external and internal cues to promote cellular homeostasis. The Golgi apparatus is essential for maintaining normal cell physiology since it supports cell survival, promoting cell proliferation, and facilitating cell-cell communication and migration. These roles are partly influenced by established Golgi functions, such as post-translational modifications, lipid production, intracellular trafficking, and protein secretion (Rossini M., et al., 2021, Febs J., 288(3):740-755). Since intracellular organelles are tightly regulated under various stress conditions, it was hypothesized that Golgi apparatus disruption under oxidative stress could alter lipid and protein modification, packaging, and transport, resulting in sub-optimal anti-tumor T cell function.

Disruption of Golgi architecture and functions, termed Golgi stress, has been previously shown to alter redox balance and affect cell survival (Alborzinia H., et al., 2018, Communications Biology, 1(1):210). Golgi stress inducers, including monensin and brefeldin A, have been widely shown to impair Golgi structure and function. These Golgi stressors have been shown to upregulate cystathionine γ-lyase (CSE) and endogenous H₂S generation, whereas inhibition of the CSE/H₂S system results in increased susceptibility to Golgi stress (Zhang Y., et al., 2020, Antioxidants & Redox Signaling, 32(9):583-601). Thus, it was hypothesized that treating T cells with exogenous H₂S would overcome Golgi stress and restore Golgi apparatus function to enhance anti-tumor T cell response. The role of H₂S in biological processes has increasingly become the focus of research in recent years. A particular focus has been on the cytoprotective and antioxidant properties that H₂S appears to have in cells that are exposed to high levels of oxidative stress (Johansen D., et al., 2006, Basic Res Cardiol., 101(1):53-60; Hu L. F., et al., 2008, Pflugers Arch., 455(6):971-8; Bian J. S., et al., 2006, J Pharmacol Exp Ther., 316(2):670-8; Sivarajah A., et al., 2006, Shock, 26(2):154-61; Elrod J. W., et al., PNAS USA, 104(39):15560-5; Fiorucci S., et al., 2005, Gastroenterology, 129(4):1210-24; Marutani E., et al., 2015, J Am Heart Assoc., 4(11); George T. J., et al., 2012, J Surg Res., 178(2):593-600; Aslami H., et al., 2013, PLOS One, 8(5):e63497; Hu L. F., et al., Aging Cell, 9(2):135-46). It was found that with traditional T cell activation methods, T cells dramatically upregulate H₂S production; however, as T cells continue to proliferate, this initial increase in H₂S returns to baseline levels. These results initially suggested to us that H₂S signaling may play an important role in supporting T cell activation and function and that sustained H₂S signaling could produce robust anti-tumor T cells. Indeed, anti-tumor T cells expanded with exogenous H₂S or overexpressing Cbs to increase endogenous H₂S production produced T cells capable of producing high levels of cytolytic cytokines and sustained levels of protein translation upon TCR stimulation.

These data demonstrate that H₂S is an important immunomodulatory signaling molecule that can be used to alter multiple factors in T cells to enhance their anti-tumor capacity and that this approach can be employed to program TILs and genetically modified T cells with potent anti-tumor phenotype. Treating T cells with H₂S donors or increasing endogenous production of H₂S supports previously established signatures of robust anti-tumor T cells, such as enhanced stemness, increased mitochondrial function, and reduced susceptibility to oxidative stress and ER stress upon chronic antigen stimulation. These data also highlight that the increased Tcm phenotype observed in H₂S treated T cells can be attributed to enhanced NAD+ levels, through the NAD+-Sirt1-Foxo1 axis (Chatterjee S., et al., 2018, Cell Metab., 27(1):85-100.e8; Hess Michelini R., et al., 2013, J Exp Med., 210(6):1189-200). Importantly, Foxo1 has also been recently shown to be important for determining CAR-T cell memory phenotype and function (Chan J. D., et al., 2024, Nature, 629(8010):201-210; Doan A. E., et al., 2024, Nature, 629(8010):211-218). Thus, it is likely that H₂S acts at multiple levels to render a “stress-free” Tcm phenotype that results in improved persistence in vivo upon adoptive transfer to bring improved tumor control.

In other cell types, H₂S has been shown to reduce ER stress, particularly in the context of oxidative stress (Wu J., et al., 2021, Int J Mol Med., 47(4); Wang C. Y., et al., 2017, Mol Med Rep., 16(3):3587-3593). A similar effect of H₂S in T cells, both in reducing overall oxidative stress and preventing ER stress upon chronic antigen stimulation was observed. Interestingly, these data show that dysfunction within the Golgi apparatus is another critical factor that needs to be considered when generating tumor reactive T cells for adoptive therapies, and that H₂S treatment during the expansion process can be used for effective programming. However, it must be noted that given the inter-dependence of organelle function in shaping the cellular response, a limitation of this study remains in determining the sole role of Golgi stress in altering the immune response when mitochondria or ER stress are also impacted. Nonetheless, these findings offer novel insight into the role of H₂S signaling in regulating both ER and Golgi network stress in T cells and offer new therapeutic strategies for improving anti-tumor T cell response.

While the ER and its associated ribosomes are responsible for synthesizing and folding proteins, the Golgi apparatus is a closely associated organelle responsible for further modification and sorting of synthesized proteins received from the ER. The status of the Golgi apparatus in T cells was of particular interest, given the importance of Golgi processing of the secreted factors that are required for T cell effector function. Similar to ER stress, multiple studies have shown that cells can also experience Golgi stress, characterized as a fragmentation of the Golgi apparatus and an inability to process proteins properly (Machamer C. E., et al., 2015, Frontiers in Neuroscience, 9; Taniguchi M., et al., 2017, Cell Struct Funct., 2(1):27-36; Sengupta D., et al., 2011, Annual Review of Cell and Developmental Biology, 27(1):57-77). Interestingly, studies have recently shown that H₂S is an important protective regulator of Golgi stress (Zhang Y., 2020, Antioxidants & Redox Signaling, 32(9):583-601). It was found that Golgi stress was a characteristic of exhausted T cells and that treatment with H₂S could reduce Golgi stress in anti-tumor T cells. These data also demonstrate an important role for the thiol-specific peroxidase Prdx4 in regulating ER and Golgi stress in anti-tumor T cells. Intriguingly, Prdx4 reactive cysteines are particularly susceptible to oxidation, rendering Prdx4 inactive when H₂O₂ levels are high (Wang X., et al., 2011, Biochemical Journal, 441(1):113-118). Strategies to target and selectively reduce the functional cysteine residues of Prdx4 and other molecules that regulate Golgi function will have the potential for high translational value to optimize immunotherapy. These findings identify Golgi stress as a novel therapeutic target in cancer immunotherapy and identify H₂S-based therapy as a potential strategy for mitigating Golgi stress to enhance anti-tumor immunity.

Further, it is of significant interest to the scientific and medical communities to identify simple phenotypic attributes of potent anti-tumor T cells. For example, Sukumar et al earlier demonstrated that T cells with low mitochondrial membrane potential as measured with TMRM dye had robust anti-tumor capacity, and that T cells with low mitochondrial membrane potential could be sorted and used for ACT to produce durable tumor control (Sukumar M., et al., 2016, Cell Metab., 23(1):63-76). Similarly, it is shown that T cells with high and low Golgi mass have distinct functionality profiles, and that sorting on Golgi^{hi} cells produces a subset of T cells with superior ability to control tumors. Whether it is increased expression of key cell signaling molecules in Golgi^{hi} fraction resulting from asymmetric cell division and distribution that contributes to long-term maintenance of T cell function and control (Chang J. T., et al., 2007, Science, 315(5819):1687-91) or if it is reduced activity of Mgat5-dependent N-glycan branching that lowers T cell threshold of activation and leads to increased functionality (Demetriou M., et al., 2001, Nature, 409(6821):733-9) will need to be dissected in future. Regardless, this approach to cell selection will likely be broadly applicable to multiple forms of ACT for treating cancer, including TIL and CAR-T therapy.

Overall, it is of critical importance to continue to identify novel therapeutic strategies for enhancing the ability of the immune system to control and eliminate tumors. An improved understanding of regulation of the T cell immune response at the organelle-level can help devise effective anti-tumor therapies focused on reducing organelle stress, limiting organelle damage, improving inter-organelle crosstalk, and restoring organelle homeostasis, and could be useful to improve immunotherapy options. Ultimately, it is determined that H₂S signaling plays a key role in immune regulation at multiple levels, including reduction of Golgi stress in anti-tumor T cells, which can be used to boost anti-tumor immunotherapeutic strategies.

### Materials and Methods

### Mice

C57BL/6, B6-Rag−/−, Pmel, NSG, and Cbs−/−mice were obtained from Jackson Laboratory (Bar Harbor, ME). Animals were maintained in pathogen-free facilities, and experimental procedures were approved by the Institutional Animal Care and Use Committees of Medical University of South Carolina, Charleston (approval #IACUC-2018-00628-1). For tumor experiments, an equal number of age- and gender-matched (both male and female) mice were randomly assigned for the experiments when they were between 8-10 weeks old. No influence of sex on the result of the studies was observed.

### Cell Lines

B16-F10 and Jurkat cells were obtained from American Type Culture Collection (ATCC), suggesting male origin. Raji cells (ATCC #CCL-86).

### Generation of Prdx4 Knockout and Prdx4 Mutant Cells

Jurkat cells were transduced with human Prdx4 shRNA lentiviral particles expressing a puromycin resistance gene. Puromycin was added to the culture media to selectively expand the transduced Jurkat cells. Prdx4 knockdown was confirmed by RT-PCR and western blot analysis. To generate the Prdx4 mutant plasmid, primers were designed based on the coding sequence of the canonical gene of interest (Prdx4). The coding sequence was converted into the amino acid codon sequence using Expasy to mutate the amino acid of interest. Roughly 15-20 amino acids upstream and downstream of the mutated codon were selected, and the New England Biolabs Tm Calculator was used to adjust the primer length, projected annealing temperature, and GC content. The Harvard Reverse Complement Tool was used to produce the reverse primer sequence. Primers were ordered from Integrated DNA Technologies, including 5′-phosphorylation for plasmid ligation. The template plasmid (containing the wild-type gene of interest for mutation) and the primers were then used with the QuikChange XL Site-Directed Mutagenesis kit (Agilent Technologies, #200516) per the manufacturer's instructions. The successful mutation was confirmed via sequencing of the plasmid (Genewiz, Azenta Life Sciences).

### Overexpression of Prdx4

To generate the lentiviral particle containing prdx4 plasmid, 293T Lenti-X cells were seeded in complete DMEM media overnight in 10 cm tissue culture plate. The next day, 4 hours prior transfection, the cells were treated with 25 μM chloroquine. Following chloroquine treatment, cells were transfected with 10 μg of either Prdx4 plasmid or mock plasmid and 7.5 μg of psPAX2 packaging plasmid and 2.5 μg of pMD2.G envelope plasmid through lipofectamine 3000 according to manufacturer's protocol. The next day, media was replaced with fresh complete DMEM media and allowed them to grow 24 hr. The next day, media containing virus particles were collected and filtered through a 0.45 μm syringe filter. Supernatants containing viral particles of either mock or Prdx4 insert were diluted at 1:1 ratio with fresh complete IMDM before mouse T cell transduction. T cells were collected from spleen of healthy Pmel mouse, and 1×10⁶/ml cells were transduced with diluted viral supernatant by spinoculation method in presence of protransdusin at 2000 rcf at 32° C. for 2 hrs. 24 hrs later, cells were collected, washed and checked for GFP expression before use for further experimental analysis.

### T Cell Differentiation

Naïve total T cells were purified from the total splenocytes of 6-9-week-old C57BL/6 mice, first by incubating the cells with biotinylated anti-CD19, anti-Gr1, Anti-Mouse TER-119, anti-CD11b, anti-CD11c, anti-NK1.1, anti-CD25, anti-CD105 (cell signaling technology), followed by negative selection with streptavidin magnetic particles (BD Biosciences). Purified T cells were then activated with soluble anti-CD3 (1 μg/ml) and anti-CD28 (1 μg/ml) in the presence of 100 IU/mL IL2. Total splenocytes from 6-9-week-old Pmel transgenic mice (bearing Class-I restricted CD8+ T cells) were activated with 1 ug/mL gp100 melanoma antigen in the presence of 100 IU/mL IL2. Within experiments, mice were age and sex-matched. T cells were cultured in IMDM media supplemented with 10% FBS, 4 mM L-glutamine, 100 U/ml penicillin, 100 μg/ml streptomycin, 55 μM beta-mercaptoethanol under 7% CO2, atmospheric oxygen at 37° C. in a humidified incubator. T cells were re-stimulated to evaluate intracellular cytokines by flow cytometry either with PMA/ionomycin for four hours or soluble anti-CD3 (1 μg/ml) and anti-CD28 (1 μg/ml) or with gp100 melanoma antigen for 6 hours in the presence of Golgi inhibitors. In some experiments, in vitro differentiated T cells were either treated with the vehicle control or H₂S donor GYY4137 (0.5 mM)

### Retroviral Transduction

For CD19-CAR-T generation, human PBMCs were obtain from healthy donors by Ficoll gradient spin and activated for 3 days with soluble anti-CD3 antibody (Okt-3, 1 ug/mL). For Cbs-overexpression, freshly isolated Pmel T cells were activated with gp100 peptide (1 ug/mL) for 3 days. CD19-CAR-T and CBS-CD19-CAR-T viral supernatant was generously gifted by Dr. Mike Nishimura (Loyola University Chicago). Cbs viral supernant for Pmel transduction was generated using Cbs human tagged ORF clone (Origene #RC201755L4). After 3 days of activation, T cells were plated at a concentration of 2×106 cells/mL in complete media onto non-tissue-culture-treated 24-well plates (USA Scientific) coated with Retronectin. 1 mL of viral supernatant was added on top of the T cells, and the plate was spun at 2,000g for 2 hr and 32° C. Post-spin, 1 mL of media was removed and replaced with fresh media containing 200 IU/ml IL-2 before the cells were incubated overnight. The cells were collected, washed, and plated the following day for use in further experiments.

### Adoptive T Cell Protocol

B16-F10 (0.3×106) melanoma tumor cells were injected subcutaneously (s.c.) into left flank of 8-10-week-old C57BL/6 or Rag−/− mice. After tumor establishment, recipient mice were injected (i.p) with cyclophosphamide (4 mg/mice) before adoptively transferring (i.v.) either Pmel, Pmel-Cbstd, or TILs (1×106). After adoptive T cell transfer, recipient mice were given IL2 (50,000 U/mouse; i.p) for three consecutive days. Raji cells (0.5×106) were injected s.c. into left flank of 8-10-week-old NSG mice. After tumor establishment, CD19-CAR-T cells (5×106) were adoptively transferred (i.v.). After adoptive T cell transfer, recipient mice were given IL2 (50,000 U/mouse; i.p) for three consecutive days. For all tumor control experiments, mice were randomly assigned to treatment groups and labeled using coded ear punch. Tumor measurements were then conducted in a blinded fashion until final analysis.

### In Vitro TME Exhaustion Assay

B16-F10 (0.3×106) melanoma tumor cells were injected subcutaneously (s.c.) into left flank of 8-10-week-old C57BL/6 mice. Once the tumors reached a size of approximately 150 mm2, the mice were euthanized and the tumors were removed. The tumors were then processed into single cell suspension using a mouse tumor dissociation kit (Miltenyi, #130-096-730). The tumor cell suspension was then plated in 6-well plates with IMDM media supplemented with 10% FBS, 100 U/ml penicillin, 100 μg/ml streptomycin, and 55 μM beta-mercaptoethanol under 7% CO2, atmospheric oxygen at 37° C. in a humidified incubator. After 5 days of culture without changing the media, the supernatant was removed from the wells and spun down to remove any tumor cells. 3-day activated Pmel T cells were then resuspended in the tumor supernatant along with 0.1 ug/mL of gp100 antigen. To promote chronic antigen stimulation, 0.1 μg/mL gp100 was added every day for 4 days. After 4 days of culture with tumor supernatant and chronic stimulation, the T cells were removed and analyzed for expression of exhaustion markers and functional assays.

### Flow Cytometry

Staining for cell surface markers was performed by incubating cells with the antibody at 1:200 dilutions in FACS buffer (0.1% BSA in PBS) for 30 min at 4° C. For intracellular cytoplasmic proteins, surface markers were stained before fixation/permeabilization (BD Cytofix/Cytoperm Kit, BD Biosciences, San Jose, CA). For staining of transcription factors, cells were stained with surface markers and fixed/permeabilized with a FoxP3 staining buffer set (eBioscience, San Diego, CA). For Cbs, pIRE1α, pPERK, and ATF4 intracellular staining, surface markers were stained before fixation/permeabilization, followed by primary unconjugated antibody staining and subsequent incubation with fluorochrome-conjugated secondary antibody (Jackson ImmunoResearch Laboratories, West Grove, PA). In addition, MitoTracker Red (Cell signaling technology #9082), LIVE/DEAD™ Fixable Yellow Dead Cell Stain Kit (Invitrogen #L34959), DCFDA dye (Abcam #ab113851) and WSP-1 dye (MCE #HY-124409) were used to evaluate mitochondrial mass, cell viability, cellular ROS, and H₂S production respectively following manufacturer's protocol. Samples were acquired on LSRFortessa and analyzed with FlowJo software (Tree Star, OR).

### Immunoblotting

For evaluation of the protein level, cell pellets were washed in PBS and lysed in RIPA buffer (Thermo Fisher Scientific, Waltham, MA), including protease/phosphatase inhibitors, vortexed, and incubated for 20 minutes on ice. Cell lysates were then centrifuged at 12,000 rpm for 15 min at 4° C. The supernatants were collected, and proteins were quantified with a BCA protein assay kit (Thermo Fisher Scientific, Waltham, MA). For immunoblot analyses, 20 μg of protein lysates per sample were denatured in 4× Loading dye and boiled using a heating block at 95 degrees for 10 minutes before loading to SDS gradient gels 4%-20% (Bio-Rad Criterion, 1h runs). Gels were semi-dry transferred onto PVDF, and the membranes were blocked with 3% milk in 0.1% TBST. The membraneNext, the membrane was probed with the following primary antibodies: anti-Prdx4 (Proteintech, 10703-1-AP), anti-eIF2a (Cell Signaling Technology, #9722), or anti-β-Actin (Signaling Technology, #4967L) overnight at 4° C. followed by one-hour incubation with HRP-conjugated secondary antibody (Cell Signaling Technology, Danvers, MA) and using a Clarity Western ECL Substrate (Bio-Rad, Hercules, CA).

### Real-Time Quantitative-PCR

Total RNA was extracted from pellets of the indicated T cell subsets (2×106 cells) using Trizol reagent (Life Technologies, Grand Island, NY). cDNA was generated from 1 μg total RNA using iScript cDNA Synthesis Kit (BioRad, Hercules, CA). SYBR Green incorporation quantitative real-time PCR was performed using an SYBR Green mix (Biorad, Hercules, CA) in the CFX96 Detection System (BioRad, Hercules, CA). The expression of different genes was quantified relative to Actb. For RT-PCR arrays, RT2 Profiler PCR Arrays (Qiagen) were used according to the manufacturer's instructions.

### RNA-Seq Analysis

Cells were immediately pelleted by centrifugation at 4° C. and resuspended in 1 mL Trizol. RNA concentration was measured using a NanoDrop 8000. RNA quality was assessed using an Agilent 4200 TapeStation and RINe values ranged from 9.7-10. Total RNA (250 ng) was used the construction of libraries with the New England Biolabs NEBNext® Poly(A) mRNA Magnetic Isolation Module (Cat #7490L) and Ultra II Directional RNA Library Prep Kit for Illumina (Cat #7760L) according to the manufacturer's instructions. Dual-indexed libraries were pooled to and sequenced at VANTAGE (Vanderbilt University Medical Center) on an Illumina NovaSeq 6000 (S4 flow cell) to a depth of approximately 25 million paired-end 150 bp reads per library. Reads were aligned to the mouse mm10 reference genome using STAR (v2.7.1a). Only uniquely mapped reads were retained for further analyses. Quality control metrics were assessed by Picard tool (http://broadinstitute.github.io/picard/). Gencode annotation for mm10 (version M25) was used as reference alignment annotation and downstream quantification. Gene level expression was calculated using featureCounts (v2.0.1). Counts were calculated based on protein-coding genes from the annotation file. Counts were normalized using counts per million reads (CPM). Genes with no reads in either Control or Treated samples were removed. To infer potential experimental confounders, surrogate variables were calculated using the sva package in R. Differential expression analysis was performed in R using DESeq2 (v1.34) with the following model: gene expression˜Treatment+nSVs. Log 2 fold changes and P-values were estimated. P-values were adjusted for multiple comparisons using a Benjamini-Hochberg correction (FDR). Differentially expressed genes where consider for FDR<0.05. Mouse Gene ID were translated into Human Gene ID using biomaRt package in R. The functional annotation of differentially expressed genes was performed using clusterProfiler (v4.2). A Benjamini-Hochberg FDR (FDR<0.05) was applied as a multiple comparison adjustment.

### Transmission Electron Microscopy (TEM)

The cells were pelletized and fixed in 2% Phosphate Buffered Glutaraldehyde for one hr. Next, the pellets were rinsed in 0.1M Phosphate Buffered Rinse and then postfixed in 2% Aqueous Osmium Tetroxide for one hr. After rinsing in distilled water, the pellets were dehydrated through a series of graded Ethyl Alcohol; 50% ETOH for 15 min, 70% ETOH for 15 min, 95% ETOH for 15 min, and finally twice with 100% ETOH for 15 min each. The dehydrant was removed using the intermediate fluid, Propylene Oxide, one change of 10 min each. Next, the pellets were infiltrated with a 1:1 solution of propylene oxide and Embed 812 (Electron Microscopy Sciences, Ft. Washington, PA) for one hr. The infiltration was continued using a 1:2 solution of propylene oxide and Embed 812 overnight. The pellets were embedded in Embed812 the following day and polymerized in a 60° C. oven for 48 hr. Preliminary ½-micron sections were cut and stained with Toluidine Blue and examined using a light microscope. Then with the cell types identified, the 70 nm thin sections are cut and stained with uranyl acetate and led citrate and allowed to dry. The sections are viewed on the JEOL 1010, and images are taken with a Hamamatsu electron microscope camera.

### Confocal Microscopy

Alexa Fluor 488 (anti-Giantin) and Alexa Fluor 647 (anti-TOMM20) fluorescence were imaged in a Zeiss LSM 880 NLO inverted laser scanning confocal microscope (Thornwood, NY) using a 63× 1.4 N.A. plan-apochromat oil immersion lens. Alexa Fluor 488 and Alexa Fluor 647 were excited at 488 nm and 633 nm, respectively. Emitted light was detected with an Airyscan super-resolution detector at BP 495-550 nm for Giantin label and LP 654 nm for TOMM20. Z-stack Airyscan images were processed using the Huygens Professional deconvolution and image analysis software (Scientific Volume Imaging, The Netherland). After images were deconvolved using Huygens' Deconvolution Express (Standard Profile) that determines optimal parameters, 3D surface rendering of deconvolved images with watershed augmentation using Huygens' Surface Renderer was performed.

### Metabolomics

Different metabolites' intracellular levels were determined by performing comprehensive hydrophilic metabolites analysis using LC/MS platform (Metabolomics Core Facility, Northwestern University). Data were then analyzed using MetaboAnalyst software. Samples were loaded equivalently across the platform and normalized to Bradford values before statistical analysis.

### Extracellular Flux Assays

Oxygen consumption rate (OCR) and extracellular acidification rate (ECAR) were determined using the Seahorse Xfe96 analyzer (Agilent Technologies, Santa Clara, CA). Briefly, T cells (0.5×10⁶/well) were plated on a Cell-Tak coated Seahorse culture plate for 30 min. OCR, a measure of OXPHOS, was analyzed under basal condition, or in response to 1.0 μM oligomycin, 1.0 μM fluoro-carbonyl cyanide phenylhydrazone (FCCP) and 2 μM rotenone, plus 100 nM antimycin A. ECAR, a measure of glycolysis, was measured under basal conditions and in response to glucose (5.5 mM), Oligomycin (1.0 μM), and 2-deoxyglucose (2-DG) (100 mM). All reagents were purchased from Sigma-Aldrich, St. Louis, MO.

### Protein Translation Assay

Pmel T cells were re-stimulated with gp100 peptide (0.1 μg/mL) and Click-iT™ Plus OPP Alexa Fluor™ 647 Protein Synthesis Assay Kit (ThermoFisher Scientific #C10458) was used to measure protein translation using flow cytometry following the manufacturer's protocol.

### Multispectral Imaging Flow-Cytometry (IFC) Analysis

T cells were stained with conjugated for surface markers as described in the figure legends followed by fixation and permeabilization with BD Cytofix/Cytoperm Kit (BD Biosciences, 554722). The Golgi was labeled using anti-Giantin antibody (Abcam, ab80864) as the primary antibody followed by secondary antibody staining with anti-Rabbit IgG conjugated to Alexa Fluor 488 (ab150077). The cells were then imaged using ImageStreamX mark II imaging flow-cytometer and analyzed using IDEAS 6.2. Spectral overlap was compensated for using single-stain controls. Image analysis for Golgi area was performed using the protocol established by Eisenberg-Lerner et al.44 First, cells were gated on single cells using the area and aspect ratio features, and then gated on focused cells using the Gradient RMS feature. Golgi area was calculated using the anti-Giantin fluorescence signaling, using on the Threshold_50 mask that includes the 50% highest intensity pixels of the Golgi staining, with a mask defined as Area Threshold 50 was considered as the Golgi area to compare between the relevant conditions.

### Proximity Ligation Assay (PLA)

PLA assays were performed using NaveniFlex Cell MR RED (Cayman, #39505) according to the manufacturer's instructions. Anti-Prdx4 (Proteintech, 10703-1-AP) and anti-Giantin (Abcam, ab37266) were used as primary antibodies. T-cells were fixed with 4% PFA for 15 minutes then permeabilized with 0.1% Triton X for another 15 min at room temperature. PLA blocking agent was then used to block non-specific binding, and the cells were then incubated overnight in the presence of antibodies of interest. After washing, secondary PLA probes conjugated to oligonucleotides were added to the cells, then a ligase was added to the samples to ligate the oligonucleotides that are in close proximity. DNA rolling-circle amplification was then performed using the PLA polymerase in the presence of fluorescence-bound oligonucleotide probes, which then yielded a fluorescent signal only where the two targets of interest are interacting in close proximity. Cells were imaged using the Olympus FV10i laser scanning confocal microscope. Signal quantification was performed using the Duolink in Situ Image Tool software.

### Isolation of Tumor-Infiltrating T Cells

To obtain tumor-infiltrating T cells (TILs) from subcutaneously established solid B16-F10 melanoma-bearing mice, tumors were excised, chopped finely using tweezers and scissors, and then digested with 2 mg/ml collagenase type IV (Stemcell Technologies, Vancouver, BC) for 45 min. The tumors were filtered through 70 cell strainers (BD Biosciences, San Jose, CA). The cell suspension was washed in culture medium twice by centrifugation at 1500 rpm for 10 min at 4° C. After the second wash, the cells were re-suspended in 6 ml PBS and layered carefully over 3 ml Ficoll-paque (GE Healthcare) followed by centrifugation at 1500 rpm for 30 min at room temperature. The enriched TILs obtained at the interface as a thin buffy layer were washed with PBS twice and finally re-suspended in FACS staining buffer for further staining procedures.

### Generation of Bone Marrow Derived Dendritic Cells

To obtain bone marrow, mice were sacrificed by CO2 inhalation. Femoral bones were removed, and all remaining tissue was dissected off the bone. The ends of each bone were cut off and the bone marrow was flushed from the center of the bone. Bone marrow cells were then cultured on non-tissue culture treated 6-well plates in complete media for 7 days with 10 ng/ml GM-CSF and 10 ng/ml IL-4 to generate dendritic cells. The media was changed every two days. Floating cells were removed, and the loosely adherent cells were considered to be dendritic cells. FACS analysis was then performed to confirm the successful generation of dendritic cells.

### N-Linked Glycosylation Profiling of T Cells by Mass Spectrometry Imaging

Antibody-based analysis of N-linked glycosylation was performed as fully described by Dressman et al.47,71 In brief, amine-reactive slides were coated with antibodies at 200 ng per 1.5 μL spot and incubated at room temperature for 1 h in a preheated humidity chamber. Bound antibodies were washed with 0.1% octyl-β-d-glucopyranoside in PBS (PBS-OGS) for 1 min, followed by blocking in 100 mM ammonium bicarbonate solution pH 8 for 30 min. Antibodies were then deglycosylated by adding 100 μL of 10 μg/mL PNGase F PRIME diluted in HPLC grade water into each well and placed back into the humidity chamber and incubated at 37° C. for 2 h. Following deglycosylation, antibody arrays were washed with PBS-OGS (3 min×3) with gentle shaking followed by PBS washes (3×) and a water wash (1 min). T cells were washed (3×) in FACS buffer and resuspended in FACS buffer. 100 μL of cell suspension was added to each well. Cell capture was performed at 4° C., shaking at 250 rpm for 1 h. The 24-well module was then removed and the slide was placed in a slide mailer containing 10% neutral buffered formalin for 20 min. After 20 min, the slide was removed and placed in PBS at RT for up to 1 week before further processing. Sialic acid stabilization and derivatization were performed via a slide-based sequential amidation-amidation reaction with dimethylamine and propargylamine, termed AAXL (amidation-alkyne Xtra linker). To release N-glycans from captured cells, PNGase F Prime (0.1 μg/μL in HPLC water) was sprayed onto the slide using an M5 TM-Sprayer (HTX Technologies). Slides were incubated for 2 h at 37° C. in a preheated humidity chamber. MALDI matrix α-cyano-4-hydroxycinamic acid (CHCA, 7 mg/mL in 50% acetonitrile/0.1% trifluoracetic acid) was sprayed using the same M5 TM-sprayer. Two passes of ammonium phosphate monobasic (5 mM) were sprayed across the slide to reduce matrix clustering and improve the signal. N-glycan imaging was conducted using a timsTOF-flex MALDI-QTOF mass spectrometer (Bruker) operated in positive ion mode at a m/z range of 700-4000. Images were collected using a SmartBeam 3D laser that operated at 10,000 Hz using the M5 small smart beam setting at a laser spot size of 100 μm run at a raster of 150 μm. 600 laser shots per pixel were collected with an ion transfer time of 120 us, a prepulse storage of 25 us, a collision radio frequency of 4000 Vpp, a multipole radio frequency of 500 Vpp, and a collision cell energy if 25 eV.

### Proteome-Wide Analysis of Reactive Cysteine Thiols by LC-MS/MS-Based Proteomics

Cells were treated+/−H₂S for 3 days of activation followed by 3 days of expansion. For analysis of differentially reactive cysteine residues, the sample preparation and analysis were as described by Gottlieb et al. with minor modifications.72 Cells were lysed in freshly made 9 M urea, 50 mM Tris pH 8 buffer with 100 units/mL Universal Nuclease (Thermo Fisher, Pierce cat #88702). To label free thiols, lysis buffer was supplemented with either 55 mM of stable isotope labeled light (I12C2ONH4) or heavy (I13CD213CONH2) iodoacetamide (Sigma, cat #721328) for control or H₂S treated cells, respectively. Equal amounts of heavy or light labeled proteins from 3 biological replicates were combined and reducible thiols were reduced with 70 mM dithiothreitol for 45 min at 25° C. After diluting with 50 mM ammonium bicarbonate, newly released thiols were alkylated with 80 mM n-ethylmaleimide (NEM) and incubated 2 hrs at 25° C. Proteins were precipitated by adding 6 volumes of cold acetone, incubated overnight at −20° C., then centrifuged at 16,000×g for 15 min at 4° C. The pellets were dissolved in an 8 M urea in 50 mM ammonium bicarbonate and the BCA protein assay was repeated. The concentration of urea was diluted to <2 M with ammonium bicarbonate and the proteins were digested with 1:33 (enzyme: protein) of Lys-C for 2 hrs at 25° C. and subsequently with 1:33 trypsin for 16 hrs at 37° C. while mixing at 300 rpm. The resulting peptides (100 μg) from 3 combined samples were each fractionated into 6 fractions using the high pH RP spin columns according to the manufacturer's protocol (Thermo Fisher, Pierce cat #84868). Eluted peptides were dried by vacuum centrifugation. Two μg aliquots of peptides from each fraction, obtained using ZipTips with 0.6 μL C18 resin (Millipore, Burlington, MA cat #ZTC18S096), were analyzed by LC-MS/MS on an Easy-nLC 1200 coupled to a Orbitrap Fusion Lumos MS (Thermo Scientific, Waltham, MA). Pressure loaded peptides were chromatographically separated on a 75 μm×50 cm column (Acclaim PepMap RSLC C18, 2 μm, 100 Å Thermo Fisher cat. #164540) thermostated at 45° C. with a gradient of 5 to 35% solvent B in 180 min (solvent A: 5% acetonitrile, 0.2% formic acid and solvent B: 80% acetonitrile, 0.2% formic acid) at 300 nL/min. Mass spectra were acquired in data dependent mode with a 3 s cycle between each MS1 acquisition. The FTMS survey MS scan mass range was m/z 375-1575. A quadrupole isolation window of 1.6 was used for precursor ion selection. Tandem mass spectra (MS/MS) were acquired following higher energy collisional dissociation of peptide precursors with 35% collision energy. Ions were detected in the orbitrap. The automatic gain control (AGC) target value was 4×10e5 for the survey MS scan at a resolution of 60,000 at m/z 400. The AGC target value for the MS/MS scan was 5×10e4 with a maximum injection time of 22 ms. Precursors with charge states 2-7 were selected for fragmentation. Dynamic exclusion was enabled with a repeat count of 1, exclusion duration of 25 sec and 10 ppm mass tolerance.

To control for changes in protein expression, an aliquot of each uncombined, labeled protein sample was analyzed using a label free proteomic approach (MaxQuant LFQ). Proteins were digested as above, and peptides were analyzed using an U3000 nano LC system coupled to an Orbitrap Elite MS (Thermo Scientific). Peptides were loaded onto a C18 PepMap 100 (300 μm×5 mm) trap column for 10 minutes at 30 μL/min of solvent A and separated using a gradient of 5 to 40% solvent B in 180 min at 200 nL/min with a 75 μm×35 cm fused-silica column (ReproSil-Pur 120 C18 AQ 1.9 μm at 50° C. (ESI Source Solutions, Woburn, MA)) packed in house. Mass spectra were acquired in data dependent mode using a top 10 method. Each FTMS survey scan was acquired with a mass range of m/z 400-1500 in the Orbitrap followed by acquisition of the tandem mass spectra in the ion trap. A normalized collision energy of 35% was used with a 10 sec activation time. The minimal signal for triggering acquisition of MS/MS was 500. Dynamic exclusion was enabled with a repeat count of 1, repeat duration of 30 sec, and exclusion duration of 180 sec.

The combined biological replicate samples were searched in MaxQuant v2.0.1.0 (Max Planck Institute) using a reviewed mouse database containing 17,090 protein sequences downloaded from Uniprot on Nov. 30, 2021. A strategy similar to that used for SILAC experiments was used to set up the search. The labels introduced during sample lysis (either carbamidomethylation of Cys with light iodoacetamide (L-IAA) or heavy iodoacetamide (H-IAA)) were created in Andromeda within the MaxQuant platform in order to obtain ratios of Heavy/Light for the combined samples. The search was set up with a multiplicity of 2 (L-IAA and H-IAA). A database of contaminants was included in the search and a maximum of 2 trypsin missed cleavages were allowed. Methionine oxidation was used as a variable modification and no fixed modifications were defined. A decoy database strategy was used as a threshold for identifications with a false discovery rate (FDR) of 0.01 at the spectrum, peptide, and protein levels. The minimum peptide length was 7 and a minimum ratio count of 2 was required for quantitation. Match between runs was enabled. The entries from the “peptides” text file were processed in Perseus v1.6.15.0 (Max Planck Institute). The peptide list was filtered to eliminate common contaminants, entries from the reversed, decoy database, and peptides that did not contain cysteine. The H/L normalized ratios calculated by MaxQuant were log 2 transformed and median normalized. Entries were filtered to retain peptides with ratios in each of the 3 biological replicate experiments. A t-test was performed to test the null hypothesis by comparing the ratios to zero. The threshold for change in reactive thiol status was a Benjamini-Hochberg adjusted p value <0.05. For the global proteomic analysis, each of the labeled, uncombined samples (n=6) were analyzed in triplicate. The database search was performed as above with the exception of using a multiplicity of 1 and the label free quantitation algorithm. Methionine oxidation and protein N-terminal acetylation were used as variable modifications; no fixed modifications were defined since the cysteines were modified with two distinct reagents (L-IAA and H-IAA) during cell lysis and NEM during sample preparation. The protein groups text file was processed in Perseus v.1.6.15.0. The list of proteins was filtered to eliminate common contaminants and entries from the reversed, decoy database. The protein intensities were log 2 transformed and filtered to retain proteins quantified in 3 biological replicate samples of either the H₂S treated or control group. Missing values were imputed from a normal distribution with a width of 0.3 downshifted by 1.8. A t-test was performed to compare the mean log 2 intensities of proteins from H₂S treated with control. To correct for multiple hypothesis testing, a permutation based FDR of 0.01 was used as the threshold for change. Peptides and proteins were annotated with GO terms (downloaded from UniProt Oct. 19, 2021) and Reactome Pathway Names (downloaded Oct. 2, 2020) using Perseus. For visualization of the data volcano plots of the log 2 fold change in peptide or protein abundance versus the −log 10 p value were generated in Perseus.

### Statistical Analysis

All data reported are the arithmetic mean from at least three independent experiments performed in triplicate±SD unless stated otherwise. The unpaired Student t-test was used to evaluate the significance of differences observed between groups, accepting P<0.05 as a threshold of significance. Data analyses were performed using the Prism software (GraphPad, San Diego, CA), except for tumor control experiments where all calculations were performed in RStudio version 2023.06.1 using R-4.1.3. For all in vivo experiments with survival outcomes as the primary outcomes, it is expected based on preliminary data that 70% of the mice in the control group will be sacrificed by 4 weeks. For a treatment to be successful, it would be expected that only 10% would be sacrificed. A sample size of 9 mice per group provides 80% power to detect this difference with a two-sided alpha of 0.05 using a log-rank test. For all survival outcomes, Kaplan-Meier curves were used to display the results. Median survival time and corresponding 95% confidence interval were calculated for each experimental condition. A log-rank test was used to compare the outcomes across experimental conditions. For all continuous outcomes, graphical displays (e.g., bar charts) were used to demonstrate patterns of the outcomes within and across experimental conditions. Normality and variance homogeneity assumptions were assessed, and appropriate data transformations were used. All continuous outcomes were measured longitudinally from the same animal. These measures were modeled using linear mixed-effects (LME) regression, including fixed effects for experimental condition, time, and their two-way interaction; subject-specific random effects were incorporated to account for the correlation among measures obtained from the same subject over time. Linear contrasts were used to conduct group comparisons at each time point for which three or more mice were alive in each treatment group. For a given time point, p-values were adjusted for multiple comparisons using Holm's method of correction. Statistical analyses were performed in a blinded fashion with the statisticians only having access to coded group identifiers without knowledge of the corresponding treatment groups.

Example 4: Sequences

| SEQ | | |
|---|---|---|
| ID NO | Name | Sequence |
| 1 | CBS forward | GATCGCCAGAAAGCTGAAGGAG |
| 2 | CBS reverse | CCACCTCATAGGCTGTTTGCTC |
| 3 | CSE forward | GTGGGACAAGAGCCTGAGCAAT |
| 4 | CSE reverse | GGATTTCCAGAGCGGCTGTATTC |
| 5 | GCLM forward | CCTCGCCTCCGATTGAAGATG |
| 6 | GCLM reverse | AAAGGCAGTCAAATCTGGTGG |
| 7 | GCLC forward | GGACAAACCCCAACCATCC |
| 8 | GCLC reverse | GTTGAACTCAGACATCGTTCCT |
| 9 | Prdx mutant | CGGACTCGCGAAGAGGAGGCCCACTTCTAC |
| | primer forward | |
| 10 | Prdx mutant | CCACCCGCGTAGAAGTGGGCCTCCTCTTC |
| | primer reverse | |
| 11 | MGAT1 forward | CCTATGACCGAGATTTCCTCGC |
| 12 | MGAT1 reverse | TGAAGCTGTCCCTGCCCGTATA |
| 13 | MGAT5A forward | AGGCAGAACCAGTCCCTTGTGT |
| 14 | MGAT5A reverse | CTTTGTGCTGGAGCCATAAACAG |
| 15 | MGAT5B forward | CTCTTACCGCAGCCTGAGTTTC |
| 16 | MGAT5B reverse | GCAGGAAGATGCAACCATTGGC |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for identifying one or more T cells with enhanced features, comprising obtaining one or more T cells, and separating from the one or more T cells, one or more T cells possessing high Golgi mass (Golgi^{hi} T cells).

2. The method of claim 1, wherein the one or more T cells is selected from the group consisting of one or more CD4+ T cells, one or more CD8+ T cells, one or more tumor infiltrating lymphocytes (TILs), one or more memory T cells (T_{CM}), and any combination thereof.

3. The method of claim 1, wherein the separating comprises:
staining the one or more T cells with a dye that stains the Golgi apparatus and sorting the stained one or more cells via flow cytometry to obtain one or more cells in which the amount of dye within the one or more cells is above a predetermined threshold (Golgi^{hi} T cells);
sorting the one or more T cells to obtain one or more T cells in which levels of branched N-glycans are below a predetermined threshold (Golgi^{hi} T cells);
sorting the one or more T cells to obtain one or more T cells in which levels of MGAT1 (β1,6 N-acetylglucosaminyltransferase I) mRNA are above a predetermined threshold (Golgi^{hi} T cells); or
sorting the one or more T cells to obtain one or more T cells in which levels of MGAT5A/B (β1,6 N-acetylglucosaminyltransferase Va/Vb) mRNA are below a predetermined threshold (Golgi^{hi} T cells).

4. The method of claim 3, wherein the dye is Bopidy™ TR Ceramide.

5. The method of claim 3, wherein the flow cytometry is Fluorescence Activated Cell Sorting (FACS).

6. The method of claim 1, wherein the one or more T cells is isolated from a subject.

7. The method of claim 1, wherein the one or more T cells are engineered to express a chimeric antigen receptor (CAR).

8. The method of claim 1, wherein enhanced features comprise increased protein translation, resistance to T cell exhaustion, increased production of proinflammatory cytokines, increased mitochondrial mass, increased mitochondrial function, increased spare respiratory capacity, upregulation of metabolic pathways, and anti-tumor activity.

9. The method of claim 8, wherein the metabolic pathways comprise glutathione metabolism, nicotinate/nicotinamide metabolism, and the mitochondrial electron transport.

10. The method of claim 8 wherein the anti-tumor activity comprises overall tumor volume reduction, increased survival and engraftment of the one or more T cells following transplant, and superior induction of tumor cell death.

11. A composition comprising the one of more T cells with enhanced features of claim 1.

12. A method of treating cancer in a subject in need thereof, the method comprising administering to the subject the composition of claim 11.

13. A method for generating one or more T cells with enhanced features, comprising obtaining one or more T cells, and performing at least one of the following steps:
administering to the one or more T cells to an agent that releases H₂S in the one or more T cells;
administering to the one or more T cells to an agent that over-expresses cystathione β-synthase (CBS) in the one or more T cells;
administering to the one or more T cells to an agent that inhibits MGAT5 enzymatic activity,
or a combination thereof.

14. The method of claim 13, wherein the one or more T cells is selected from the group consisting of one or more CD4+ T cells, one or more CD8+ T cells, one or more tumor infiltrating lymphocytes (TILs), one or more memory T cells (T_{CM}), and any combination thereof.

15. The method of claim 13, wherein the agent that over-expresses CBS in the one or more T cells is selected from the group consisting of a plasmid, a virus, a nucleic acid molecule, and any combination thereof.

16. The method of claim 13, wherein the agent that releases H₂S is selected from the group consisting of GYY 4137 and sodium hydrogen sulfide (NaHS).

17. The method of claim 13, wherein the agent that inhibits MGAT5 enzymatic activity comprises Phostine PST3.1a.

18. The method of claim 13, wherein the one or more T cells is isolated from a subject.

19. The method of claim 13, wherein the one or more T cells are engineered to express a chimeric antigen receptor (CAR).

20. The method of claim 13, wherein enhanced features comprise increased protein translation, resistance to T cell exhaustion, increased production of proinflammatory cytokines, increased mitochondrial mass, increased mitochondrial function, increased spare respiratory capacity, upregulation of metabolic pathways, and anti-tumor activity.

21. The method of claim 20, wherein the metabolic pathways comprise glutathione metabolism, nicotinate/nicotinamide metabolism, and the mitochondrial electron transport.

22. The method of claim 20 wherein the anti-tumor activity comprises overall tumor volume reduction, increased survival and engraftment of the one or more T cells following transplant, and superior induction of tumor cell death.

23. A composition comprising the one or more T cells with enhanced features of claim 13.

24. A method of treating cancer in a subject in need thereof, the method comprising administering to the subject the composition of claim 23.

25. A method of increasing the presence of Golgi^{hi} T cells in a population of T cells, the method comprising administering to the population of T cells at least one of the following:
an agent that releases H₂S in one or more T cells of the population of T cells;
an agent that over-expresses cystathione β-synthase (CBS) in the one or more T cells in one or more T cells of the population of T cells; and
an agent that inhibits MGAT5 enzymatic activity in one or more T cells of the population of T cells.
